(19) United States
(12) Patent Application Publication
(10) Pub. No.: US 2014/0227780 A1
(43) Pub. Date: Aug. 14, 2014

(54) METHOD FOR PRODUCING MEGAKARYOCYTES AND/OR PLATELETS FROM PLURIPOTENT STEM CELLS

(71) Applicants: The UNIVERSITY OF TOKYO, Bunkyo-ku (JP); NISSAN CHEMICAL INDUSTRIES, LTD., Chiyoda-ku (JP)

(72) Inventors: Taito Nishino, Chiyoda-ku (JP); Takanori Nakamura, Minamisaitama-gun (JP); Shunsuke Iwamoto, Funabashi-shi (JP); Koji Eto, Bunkyo-ku (JP); Hiromitsu Nakauchi, Bunkyo-ku (JP); Kayoko Tsuji, Bunkyo-ku (JP)

(73) Assignees: NISSAN CHEMICAL INDUSTRIES, LTD., Chiyoda-ku, Tokyo (JP); The UNIVERSITY OF TOKYO, Bunkyo-ku, Tokyo (JP)

(21) Appl. No.: 14/349,154

(22) PCT Filed: Oct. 3, 2012
(86) PCT No.: PCT/JP12/75705

§ 371 Date: Apr. 2, 2014

(30) Foreign Application Priority Data
Oct. 3, 2011 (JP) 2011-219545

(51) Int. Cl. C12N 5/078

(52) U.S. Cl. CPC C12N 5/0644
USPC 435/377

(57) ABSTRACT

An agent for inducing production of megakaryocytes and/or platelets from pluripotent stem cells which is useful for treatment of disease accompanied by a decrease in platelets is provided. A method for producing megakaryocytes and/or platelets, including separating hematopoietic progenitor cells from the septal cells in sac-like structures produced by pluripotent stem cells, and culturing the hematopoietic progenitor cells ex vivo in the presence of a compound represented by the formula (I)

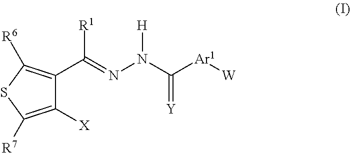

where R¹ to R⁷, W, X, Y, Z, Ar¹ and n are as defined in the description to differentiate them into megakaryocytes and/or platelets.

### TECHNICAL FIELD

The present invention relates to a method for producing megakaryocytes and/or platelets from pluripotent stem cells. In particular, it relates to a method for efficiently producing megakaryocytes and/or platelets by culturing hematopoietic progenitor cells derived from iPS cells (induced pluripotent stem cells) or ES cells (Embryonic stem cells) in the presence of a compound having a platelet expanding activity.

### BACKGROUND ART

For treatment of blood-related diseases including leukemia, it is extremely important to supply therapeutically necessary amounts of blood cells stably by cell expansion. Among blood cells, platelets are essential for blood coagulation and hematostasis and, hence, are in high demand for leukemia, bone marrow transplantation, thrombocytopenia, anticancer therapy and the like. To date, platelets have been supplied from blood collected from blood donors. However, it is sometimes difficult to supply platelets to patients stably by blood donation from donors because of the risk of virus transmission, the chronic shortage of donors and the inviability of collected platelets during long term storage. Apart from blood donation from donors, other approaches such as administration of thrombopoietin (TPO) to patients and differentiation of megakaryocytes in umbilical cord blood or myelocytes were attempted However, TPO administration to patients has not come into practical use because of generation of antibodies neutralizing TPO after TPO administration. In recent years, ex vivo platelet production techniques have been studied to replace blood transfusion by returning platelets produced ex vivo by culturing hematopoietic stem cells and hematopoietic progenitor cells into living bodies. Development of these techniques into ex vivo production of large amounts of platelets is expected to dispense with the current blood donation system and almost solve the problems of the shortage of platelet products and the virus risk. Though as the source of hematopoietic stem cells and hematopoietic progenitor cells, bone marrow, umbilical cord blood and peripheral blood are known, it is difficult to stably produce and supply large amounts of platelets from these sources, because hematopoietic stem cells and hematopoietic progenitor cells which can produce megakaryocytes and platelets can be obtained only in small numbers from these sources.

In recent years, for ex vivo platelet production, several reports have been made on efficient differentiation of hematopoietic stem cells and hematopoietic progenitor cells derived from ES cells (Embryonic stem cells) into magakaryocytes and platelets. Eto et al. demonstrated that coculture with OP9 stromal cells induces mouse ES cells to differentiate into megakaryocytes (Non-Patent Document 1). Fujimoto et al. reported that they confirmed induction of platelets by using the same system as Eto et al. (Non-Patent Document 2). Successful induction of differentiation of primate ES cells into megakaryocytes (Non-Patent Document 3) and successful induction of platelets from human ES cells (Non-Patent Document 4) were reported. However, even if production of platelets from ES cells is established to a clinically applicable level, transfusion of ES cell-derived platelets to patents still has the problem of human leukocyte antigen (HLA) compatibility (in the cases of frequent transfusions into the same patient, though not in the case of the initial transfusion).

iPS cells (Induced pluripotent stem cells) are also called artificial pluripotent stem cells or induced pluripotent stem cells and are cells derived from somatic cells such as fibroblasts which have acquired pluripotency equivalent to that of ES cells by transduction of several transcription factor genes. Mouse iPS cells were established for the first time by Yamanaka et al. by transduction of four genes, Oct3/4, Sox2, Klf4 and c-Myc, into mouse fibroblasts, using the expression of Nanog gene important for maintenance of pluripotency as a marker (Non-Patent Document 5). Later, establishment of mouse iPS cells by similar methods was reported (Non-Patent Document 6 and Non-Patent Document 7). Further, it was reported that iPS cells were established by transduction of only the three genes other than c-Myc (Oct3/4, Sox2 and Klf4) to solve the problem of tumorigenesis of iPS cells (Non-Patent Document 8). With respect to human iPS cells, Thomson et al. established human iPS cells by transduction of OCT3/4, SOX2, NANOG and LIN28 into human fibroblasts (Non-Patent Document 9). Yamanaka et al. also established human iPS cells by transduction of OCT3/4, SOX2, KLF4 and c-MYC into human fibroblasts (Non-Patent Document 9). iPS cells are expected to solve the problems with ex vivo platelet production such as insufficient quantities of hematopoietic stem cells and hematopoietic progenitor cells in bone marrow and umbilical cord blood, ethical issues and the problem of rejection in terms of using ES cells. In a study made from such a perspective, success in induction of differentiation of human iPS cells into platelets was reported (Patent Document 1), and addition of proteins such as TPO is effective for induction of differentiation into megakaryocytes and platelets is suggested.

Recent years have seen reports that low-molecular-weight compounds synthesized through organic chemistry are effective as therapeutic drugs for thrombocytopenia (Patent Documents 2 and 3) and effective for ex vivo expansion of hematopoietic stem cells (Patent Documents 4, 5, 6, 7 and 8).

### PRIOR ART DOCUMENT

### Patent Documents

Patent Document 1: WO 2009/122747
Patent Document 2: WO 2004/108683
Patent Document 3: WO 2007/010954
Patent Document 4: WO 2009/072624
Patent Document 5: WO 2009/072625
Patent Document 6: WO 2009/072626
Patent Document 7: WO 2009/072635
Patent Document 8: WO 2010/140685

### Non-Patent Documents

Non-Patent Document 1: Eto et al., Proc. Acad. Sci. USA 2002, 99: 12819-12824.
Non-Patent Document 2: Fujimoto et al., Blood 2003, 102: 4044-4051.
Non-Patent Document 3: Hiroyama et al. Exp. Hematol. 2006, 34: 760-769.
Non-Patent Document 4: Takayama et al., Blood 2008, 111: 5298-5306.
Non-Patent Document 5: Okita et al., Nature 2007, 448: 313-317.
Non-Patent Document 6: Wernig et al., Nature 2007, 448: 318-324.
Non-Patent Document 7: Maherali et al., Cell Stem Cell 2007, 1: 55-70.
Non-Patent Document 8: Nakagawa et al., Nat Biotechnol 2008, 26: 101-106.
Non-Patent Document 9: Yu et al., Science 2007, 318: 1917-1920.
Non-Patent Document 10: Takahashi et al., Cell 2007, 131: 861-872.

### DISCLOSURE OF THE INVENTION

### Technical Problem

An object of the present invention is to establish a method for obtaining megakaryocytes and platelets from pluripotent stem cells, in particular, to establish a method for obtaining megakaryocytes and platelets with stable efficiency.

### Solution to Problems

The present inventors have conducted intensive studies to solve the above-mentioned object in search for compounds capable of inducing megakaryopoiesis and thrombopoiesis from pluripotent stem cells and found out that the compounds represented by the following formula (I) have excellent megakaryopoietic and thrombopoietic activity even in the absence of TPO and that megakaryocytes and/or platelets can be produced ex vivo stably and efficiently. The present invention was accomplished on the basis of this discovery.

Namely, the present invention provides the following methods [1] to [30], megakaryocytes and/or platelets [31], blood preparation [32] and kit [33].

[1] A method for producing megakaryocytes and/or platelets, comprising culturing hematopoietic progenitor cells derived from pluripotent stem cells ex vivo in the presence of a compound represented by the formula (I), a tautomer, prodrug or pharmaceutically acceptable salt of the compound or a solvate thereof and differentiating the hematopoietic progenitor cells into megakaryocytes and/or platelets;

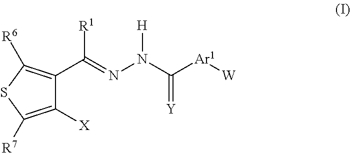

wherein W is a substituent represented by the formula (Ia) or a carboxy group:

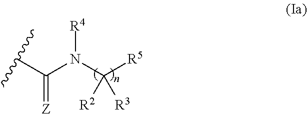

each of R¹, R², R³ and R⁴ is independently a C₁₋₁₀ alkyl group which may be substituted with one or more halogen atoms or a hydrogen atom,
n is an integer of 0, 1, 2 or 3,
R⁵ is a C₂₋₁₄ aryl group which may be substituted with one or more substituents independently represented by V¹, provided that when n is 2, R⁵ is not an unsubstituted pyridyl group,
R⁶ is a C₁₋₁₀ alkyl group which may be substituted with one or more halogen atoms or a hydrogen atom,
R⁷ is a C₂₋₁₄ aryl group which may be substituted with one or more substituents independently represented by V²,
Ar¹ is a C₂₋₁₄ arylene group which may be substituted with one or more substituents independently represented by V³,

### X is —OR²⁰,

each of Y and Z is independently an oxygen atom or a sulfur atom,
V¹ is —(CH₂)m₁M¹NR⁸R⁹, —(CH₂)m₆NR¹⁶R¹⁷, -M²NR¹⁸(CH₂)m₇R¹⁹ or —C(═O)-(piperazine-1,4-diyl)-U,
each of V², V³ and V⁴ is independently a hydroxy group, a protected hydroxy group, an amino group, a protected amino group, a thiol group, a protected thiol group, a nitro group, a cyano group, a halogen atom, a carboxy group, a carbamoyl group, a sulfamoyl group, a sulfo group, a formyl group, a C₁₋₃ alkoxy group which may be substituted with one or more halogen atoms, a C₁₋₁₀ alkyl group which may be substituted with one or more halogen atoms, a C₂₋₆ alkenyl group, a C₂₋₆ alkynyl group, a C₁₋₁₀ alkylcarbonyloxy group, a C₁₋₁₀ alkoxycarbonyl group, a C₁₋₁₀ alkoxy group, a C₁₋₁₀alkylcarbonyl group, a C₁₋₁₀ alkylcarbonylamino group, a mono- or di-C₁₋₁₀ alkylamino group, a C₁₋₁₀ alkylsulfonyl group, a C₁₋₁₀ alkylaminosulfonyl group, a C₁₋₁₀ alkylaminocarbonyl group, a C₁₋₁₀ alkylsulfonylamino group or a C₁₋₁₀ thioalkyl group,
each of M¹ and M² is independently —(C=O)— or —(SO₂)—,
m₁ is an integer of 0, 1 or 2,
each of m₂, m₃, m₄, m₅, m₆ and m₇ is independently an integer of 1 or 2,
R⁸ is a hydrogen atom or a C₁₋₃ alkyl group,
each of R⁹ and U is independently —(CH₂)m₂OR¹⁰ or —(CH₂)m₄NR¹¹R¹¹R¹², provided that when m₁ is 1 or 2, R⁹ may be any of those mentioned above or a hydrogen atom,
R¹⁰ is a hydrogen atom, a C₁₋₃ alkyl group or —(CH₂)m₃T,
each of R¹¹ and R¹² is independently a hydrogen atom or —(CH₂)m₅Q, or N, R¹¹ and R¹² mean, as a whole, a substituent represented by the formula (II):

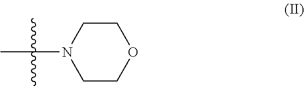 [SMILES: *N1CCOCC1]

or a substituent represented by the formula (III):

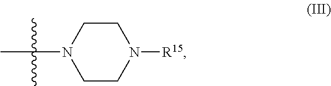

T is a hydroxy group, a C₁₋₆ alkoxy group or a C₁₋₆ alkyl group,
Q is a hydroxy group, a C₁₋₃ alkoxy group or —NR¹³R¹⁴,
each of R¹³ and R¹⁴ is independently a hydrogen atom or a C₁₋₃ alkyl group,
R¹⁵ is a hydrogen atom, a C₁₋₃ alkyl group or an amino-protecting group,
each of R¹⁶ and R¹⁷ is independently a hydrogen atom, a C₁₋₃ alkylcarbonyl group or a C₁₋₃ alkylsulfonyl group,
R¹⁸ is a hydrogen atom or a C₁₋₃ alkyl group,
R¹⁹ is a C₂₋₉ heterocyclyl group or a C₂₋₁₄ aryl group, and
R²⁰ is a hydrogen atom, a C₁₋₁₀ alkyl group which may be substituted with one or more substituents independently represented by V⁴ or a C₁₋₁₀ alkylcarbonyl group which may be substituted with one or more substituents independently represented by V⁴.
[2] The method according to [1], wherein W is a substituent represented by the formula (Ia):

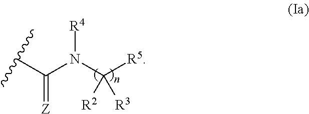

[3] The method according to [2], wherein R¹ is a hydrogen atom or a C₁₋₆ alkyl group which may be substituted with one or more halogen atoms,
each of R², R³, R⁴ and R⁶ is independently a hydrogen atom or a C₁₋₃ alkyl group,
n is an integer of 1 or 2,
Ar¹ is represented by the formula (IV):

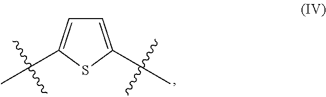 [SMILES: *c1ccc(*)s1]

R⁷ is a phenyl group which may be substituted with one or more substituents selected from the group consisting of C₁₋₁₀ alkyl groups which may be substituted with one or more halogen atoms, C₁₋₁₀ alkoxy groups, C₁₋₃ alkoxy groups substituted with one or more halogen atoms and halogen atoms,

### X is —OH, and

Y and Z are oxygen atoms.
[4] The method according to [3], wherein R², R³, R⁴ and R⁶ are hydrogen atoms.
[5] The method according to any one of [2] to [4], wherein R⁵ is a phenyl group which may be substituted with one or more substituents independently represented by V¹.
[6] The method according to any one of [2] to [4], wherein R⁵ is a C₂₋₉ heteroaryl group which may be substituted with one or more substituents independently represented by V¹.
[7] The method according to [6], wherein the C₂₋₉ heteroaryl group is a C₂₋₉ nitrogen-containing heteroaryl group.
[8] The method according to [7], wherein the C₂₋₉ nitrogen-containing heteroaryl group is selected from a 2-pyridyl group, a 3-pyridyl group, a 4-pyridyl group, a 3-pyridazinyl group, a 4-pyridazinyl group, a 2-pyrimidinyl group, a 4-pyrimidinyl group, a 5-pyrimidinyl group and a 2-pyrazinyl group.
[9] The method according to [7], wherein the C₂₋₉ nitrogen-containing heteroaryl group is a 4-pyridyl group.
[10] The method according to any one of [2] to [9], wherein V¹ is represented by any one of the formulae (V) to (XXII):

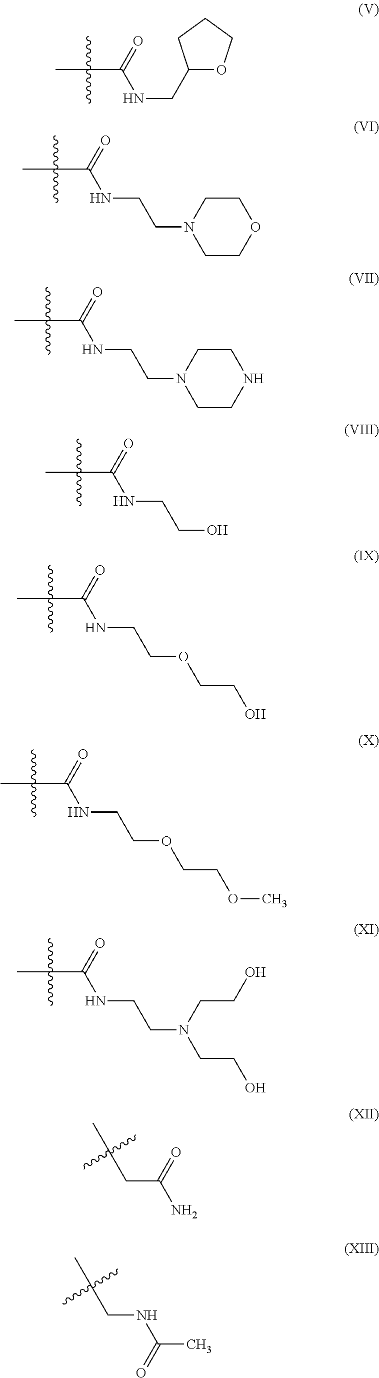

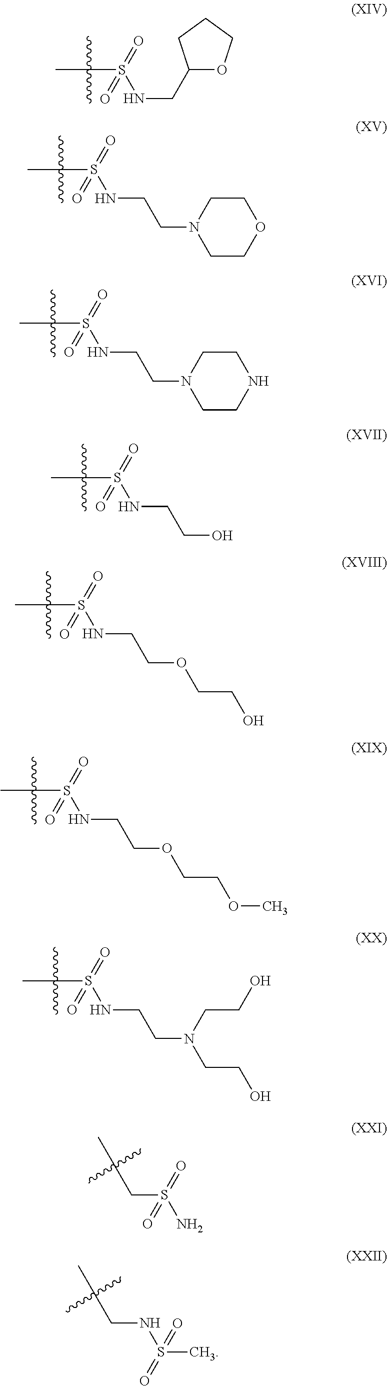

[11] The method according to [3] or [4], wherein R⁵ is a phenyl group substituted with a substituent represented by the formula (VIII):

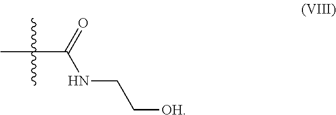 [SMILES: *C(=O)NCCO]

[12] The method according to [3] or [4], wherein R⁵ is a 4-pyridyl group.
[13] The method according to any one of [2] to [12], wherein n is an integer of 1.
[14] The method according to any one of [2] to [13], wherein R⁷ is a phenyl group substituted with one or more substituents selected from methyl groups, t-butyl groups, halogen atoms, methoxy groups, trifluoromethyl groups and trifluoromethoxy groups.
[15] The method according to any one of [2] to [13], wherein R⁷ is a phenyl group which may be substituted with one or two halogen atoms.
[16] The method according to any one of [2] to [15], wherein R¹ is a methyl group.
[17] The method according to claim 2, wherein the compound represented by the formula (I) is (E)-5-(2-{1-[5-(3,4-dichlorophenyl)-4-hydroxythiophen-3-yl]ethylidene}hydrazinecarbonyl)-N-[4-(2-hydroxyethylcarbamoyl)benzyl]thiophene-2-carboxamide, (E)-5-(2-{1-[5-(4-bromophenyl)-4-hydroxythiophen-3-yl]ethylidene}hydrazinecarbonyl)-N-[4-(2-hydroxyethylcarbamoyl)benzyl]thiophene-2-carboxamide or (E)-5-(2-{1-[5-(3,4-dichlorophenyl)-4-hydroxylthiophen-3-yl]ethylidene}hydrazinecarbonyl)-N-(pyridin-4-ylmethyl)thiophene-2-carboxamide.
[18] The method according to [1], wherein W is a carboxy group.
[19] The method according to [18], wherein R¹ is a hydrogen atom or a C₁₋₆ alkyl group which may be substituted with one or more halogen atoms,
R⁶ is a hydrogen atom or a C₁₋₃ alkyl group which may be substituted with one or more halogen atoms,
R⁷ is a C₂₋₁₄ aryl group

### X is —OH,

Y is an oxygen atom or a sulfur atom, and
Ar¹ is represented by the formula (IV):

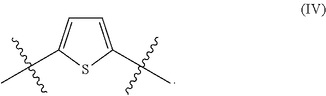 [SMILES: *c1ccc(*)s1]

[20] The method according to [19], wherein R¹ is a hydrogen atom or a C₁₋₆ alkyl group,
R⁶ is a hydrogen atom,
R⁷ is a substituent represented by any one of the formulae (A01) to (A15):

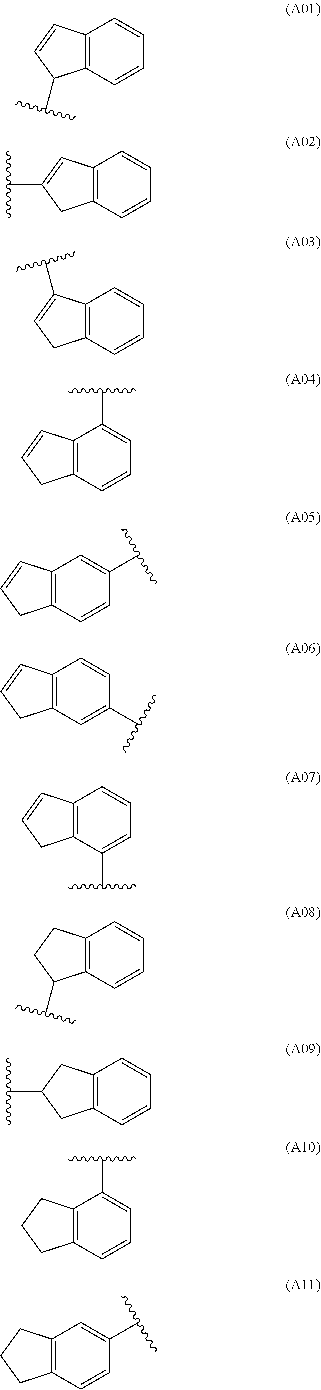

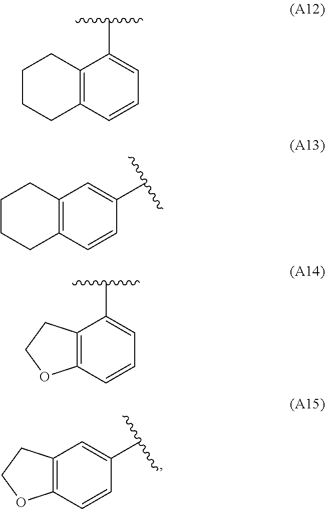

and
Y is an oxygen atom.
[21] The method according to [20], wherein R¹ is a C₁₋₆ alkyl group, and
R⁷ is a substituent represented by the formula (A11):

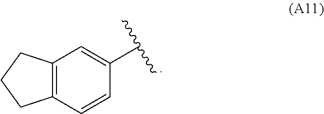 [SMILES: *c1ccc2c(c1)CCC2]

[22] The method according to [1], wherein the compound represented by the formula (I) is (E)-5-(2-{1-[5-(2,3-dihydro-1H-indene-5-yl)-4-hydroxythiophen-3-yl]ethylidene}hydrazinecarbonyl)thiophene-2-carboxylic acid.
[23] The method according to [1], wherein R¹ is a hydrogen atom or a C₁₋₆ alkyl group which may be substituted with one or more halogen atoms,
each of R², R³, R⁴ and R⁶ is independently a hydrogen atom or a C₁₋₃ alkyl group,
n is an integer of 1 or 2,
R⁵ is a phenyl group or a C₂₋₉ heteroaryl group which may be substituted with one or more substituents independently represented by V¹,
R⁷ is a phenyl group which may be substituted with one or more substituents selected from C₁₋₁₀ alkyl groups which may be substituted with one or more halogen atoms, C₁₋₁₀ alkoxy groups, C₁₋₃ alkoxy groups substituted with one or more halogen atoms and halogen atoms or a substituent represented by any one of the formulae (A01) to (A15):

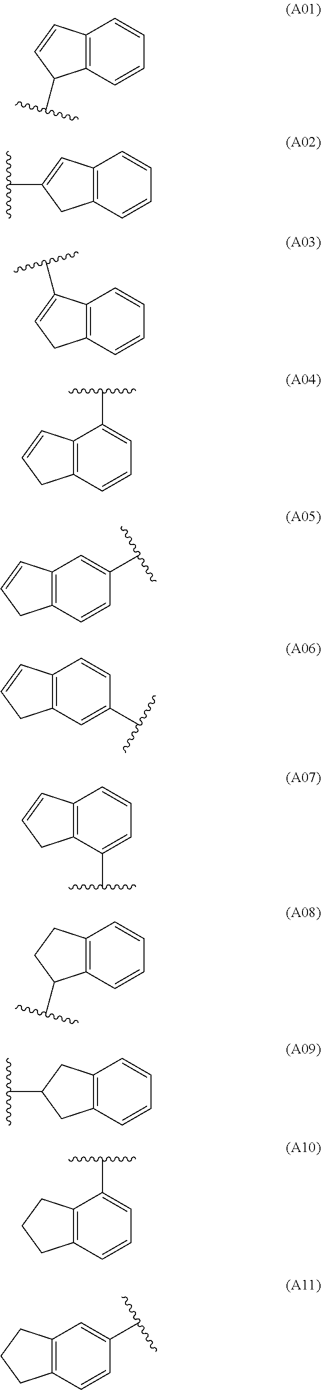

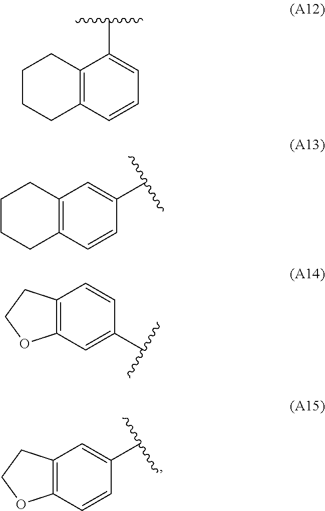

Ar¹ is represented by the formula (IV):

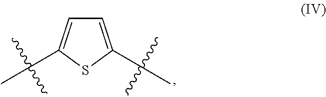 [SMILES: *c1ccc(*)s1]

### X is —OH, and

each of Y and Z is independently an oxygen atom or a sulfur atom.
[24] The method according to [22], wherein R¹ is a hydrogen atom or a C₁₋₆ alkyl group, R², R³, R⁴ and R⁶ are hydrogen atoms,
n is an integer of 1,
R⁵ is a pyridyl group, a pyrazinyl group or a phenyl group substituted with a substituent represented by the formula (VII), (VIII), (XI) or (XII):

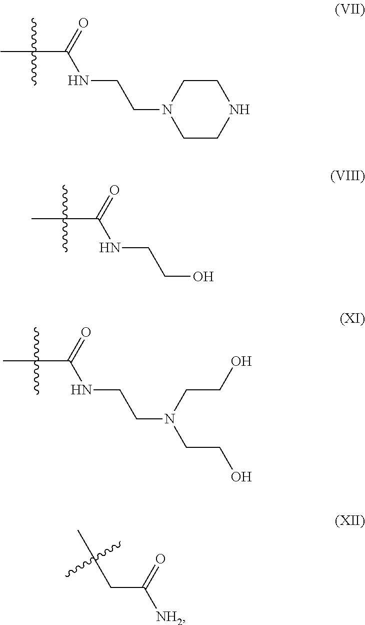 [SMILES: *C(=O)NCCN1CCNCC1 ; *C(=O)NCCO ; *C(=O)NCCN(CCO)CCO ; *CC(N)=O]

R⁷ is a phenyl group which may be substituted with one or two halogen atoms or C₁₋₁₀ alkyl groups or a substituent represented by the formula (A11), (A13) or (A15):

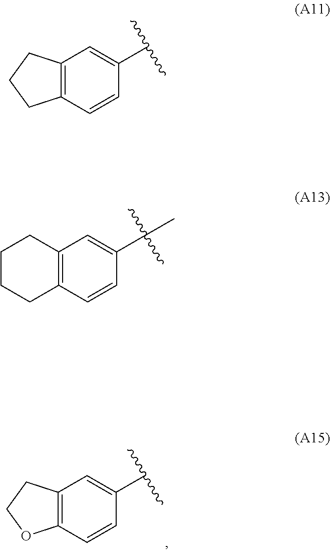 [SMILES: *c1ccc2c(c1)CCC2 ; *c1ccc2c(c1)CCCC2 ; *c1ccc2c(c1)CCO2]

and
Y and Z are oxygen atoms.
[25] The method according to [24], wherein R¹ is a C₁₋₆ alkyl group, and
R⁷ is a group represented by the formula (A11):

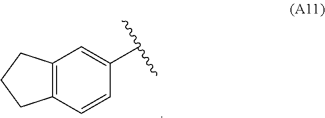 [SMILES: *c1ccc2c(c1)CCC2]

[26] The method according to [1], wherein the compound represented by the formula (I) is (E)-5-(2-{1-[5-(3,4-dichlorophenyl)-4-hydroxythiophen-3-yl]ethylidene}hydrazinecarbonyl)-N-[4-(2-hydroxyethylcarbamoyl)benzyl]thiophene-2-carboxamide, (E)-5-(2-{1-[5-(4-bromophenyl)-4-hydroxythiophen-3-yl]ethylidene}hydrazinecarbonyl)-N-[4-(2-hydroxyethylcarbamoyl)benzyl]thiophene-2-carboxamide, (E)-5-(2-{1-[5-(3,4-dichlorophenyl)-4-hydroxythiophen-3-yl]ethylidene}hydrazinecarbonyl)-N-(pyridin-4-ylmethyl)thiophene-2-carboxamide, (E)-5-(2-{1-[5-(2,3-dihydro-1H-inden-5-yl)-4-hydroxythiophen-3-yl]ethylidene}hydrazinecarbonyl)thiophene-2-carboxylic acid, potassium (E)-2-(3,4-dichlorophenyl)-4-[1-(2-{5-[(pyrazin-2-ylmethyl)carbamoyl]thiophene-2-carbonyl}hydrazono)ethyl]thiophen-3-olate, (E)-5-(2-{1-[5-(4-bromophenyl)-4-hydroxythiophen-3-yl]ethylidene}hydrazinecarbonyl)-N-{4-[2-(piperazin-1-yl)ethylcarbamoyl]benzyl}thiophene-2-carboxamide, (E)-N-[4-(2-amino-2-oxoethyl)benzyl]-5-(2-{1-[5-(3,4-dichlorophenyl)-4-hydroxythiophen-3-yl]ethylidene}hydrazinecarbonyl)thiophene-2-carboxamide or (E)-N-(4-{2-[bis(2-hydroxyethyl)amino]ethylcarbamoyl}benzyl)-5-(2-{1-[5-(4-t-butylphenyl)-4-hydroxythiophen-3-yl]ethylidene}hydrazinecarbonyl)thiophene-2-carboxamide.
[27] The method according to any one of [1] to [26], wherein the pluripotent stem cells are ES cells or iPS cells.
[28] The method according to any one of [1] to [27], wherein the hematopoietic progenitor cells derived from pluripotent stem cells are hematopoietic progenitor cells obtained from a sac-like structure formed by differentiating pluripotent stem cells into hematopoietic progenitor cells.
[29] The method according to any one of [1] to [28], wherein the hematopoietic progenitor cells derived from pluripotent stem cells have one or more introduced genes selected from oncogenes, polycomb genes, apoptosis suppressor genes and genes which suppress a tumor suppressor gene and have proliferative and/or differentiative capability enhanced by regulation of expression of the introduced genes.
[30] The method according to any one of [1] to [29], wherein the hematopoietic progenitor cells derived from pluripotent stem cells are hematopoietic progenitor cells which have one or more introduced genes selected from MYC family genes, Bmi1 genes, BCL2 family genes and genes which suppress the p53 gene expression and have proliferative and/or differentiative capability enhanced by regulation of expression of the introduced genes.
[31] Megakaryocytes and/or platelets obtained by the method as defined in any one of [1] to [30].
[32] A blood preparation containing platelets obtained by the method as defined in any one of [1] to [30], as an active ingredient.
[32] A kit for producing platelets by the method as defined in any one of [1] to [30].

### Advantageous Effect(s) of Invention

The present invention makes it possible to induce megakaryocytes and platelets from hematopoietic progenitor cells derived from pluripotent stem cells (especially, human iPS cells or human ES cells) by using the compounds represented by the formula (I), tautomers, prodrugs or pharmaceutically acceptable salts of the compounds or solvates thereof (which will be collectively referred to as specific compounds). When used in culture of hematopoietic progenitor cells derived from pluripotent stem cells, the specific compounds induce megakaryocytes and platelets more stably and more efficiently than proteins such as TPO. Namely, the method of the present invention realizes stable blood preparations containing platelets as an active ingredient.

The specific compounds are low-molecular-weight compounds obtainable by ordinary processes for organic synthesis and hence, are easy to produce under conditions which preclude microbial cell survival. Therefore, the method for producing platelet using the specific compounds can prevent contamination with an unknown pathogen or a biomaterial from an nonhuman animal more easily than conventional methods using proteins such as cytokines and growth factors obtained by gene recombination technology. Namely, platelets produced by the method of the present invention can avoid infections, contamination with foreign genes or immune response to foreign proteins. While being proteins, cytokines and growth factors can be stored or used within very narrow optimal ranges in terms of pH, temperature and ion strength, the specific compounds can be used and stored under relatively broad ranges of conditions. In addition, because the specific compounds can be produced continuously at low costs, unlike proteins, it is possible to eventually reduce treatment cost.

### DESCRIPTION OF DRAWING(S)

FIG. 1 A graph showing that megakaryocytes (CD41a⁺ CD42b⁺ cells) were expanded more remarkably in a culture of iPS cell-induced hematopoietic progenitor cells in the presence of specific compounds than in the presence of TPO. The ordinance of the graph is the number of megakaryocytes (CD41a⁺ CD42b⁺ cells) in the presence of the specific compounds relative to that in the absence of the compounds.

FIG. 2 A graph showing megakaryocytes (CD41a⁺ CD42b⁺ cells) were expanded more remarkably in a culture of iPS cell-induced hematopoietic progenitor cells in the presence of specific compounds than in the presence of TPO. The ordinance of the graph is the number of megakaryocytes (CD41a⁺ CD42b⁺ cells) in the presence of the specific compounds relative to that in the presence of TPO.

FIG. 3 A graph showing that platelets (CD41a⁺ CD42b⁺ cells) were expanded more remarkably in a culture of iPS cell-derived hematopoietic progenitor cells in the presence of specific compounds than in the presence of TPO. The ordinance of the graph is the number of platelets (CD41a⁺ CD42b⁺ cells) in the presence of the specific compounds relative to that in the absence of the compounds.

FIG. 4 A graph showing platelets (CD41a⁺ CD42b⁺ cells) were expanded more remarkably in a culture of iPS cell-derived hematopoietic progenitor cells in the presence of specific compounds than in the presence of TPO. The ordinance of the graph is the number of platelets (CD41a⁺ CD42b⁺ cells) in the presence of the specific compounds relative to that in the presence of TPO.

FIG. 5 A graph showing integrin activation (PAC-1 binding to platelets) by ADP on platelets (CD41a⁺ CD42b⁺ cells) prepared from iPS cells in the presence of specific compounds. The ordinate of the graph is the PAC-binding to the platelets relative to the PAC-binding to platelets from peripheral blood.

FIG. 6 A graph showing platelets (CD41a⁺ CD42b⁺ cells) were expanded more remarkably in a culture of ES cell-derived hematopoietic progenitor cells in the presence of a specific compound than in the presence of TPO. The ordinance of the graph is the number of platelets (CD41a⁺ CD42b⁺ cells) in the presence of the specific compounds relative to that in the absence of the specific compound.

FIG. 7 A graph showing platelets (CD41a⁺ CD42b⁺ cells) were expanded more remarkably in a culture of genetically manipulated hematopoietic progenitor cells with enhanced proliferative and differentiative capability in the presence of a specific compound than in the presence of TPO. The ordinance of the graph is the number of platelets (CD41a⁺ CD42b⁺ cells) in the presence of the specific compound relative to that in the presence of TPO.

### DESCRIPTION OF EMBODIMENT(S)

Now, the present invention will be described in detail.

The terms herein are defined as follows.

Pluripotent stem cells are cells having both pluripotency which allows them to differentiate into various kinds of cells in the body such as those in the endoderm (interior stomach lining, gastrointestinal tract, the lungs), in the mesoderm (muscle, bone, blood, urogenital) and in the ectoderm (epidermal tissues and nervous system) and self-renewal ability to proliferate through cell division while maintaining the pluripotency, and as examples, ES cells, iPS cells, embryonic germ cells (EG cells) and Muse cells may be mentioned. ES cells are pluripotent stem cells derived from an embryo at an early stage in the development of animals called the blastocysto stage. iPS cells are also called artificial pluripotent stem cells or induced pluripotent stem cells and are cells derived from somatic cells such as fibroblasts which have acquired pluripotency equivalent to that of ES cells by transduction of several transcription factor genes. EG cells are pluripotent stem cells derived from spermatogonia) cells (see: Nature. 2008, 456, 344-49). Muese cells are pluripotent stem cells separated from mesenchymal cell populations (see: Proc Natl Acad Sci USA. 2010, 107, 8639-43).

Hematopoietic stem cells are defined as cells having both pluripotency which allows them to differentiate into blood cells of all lineages and the ability to renew themselves while maintaining the pluripotency. Multipotential hematopoietic progenitor cells are cells which can differentiate into a plurality of blood cell lineages, though not into all blood cell lineages Unipotential hematopoietic progenitor cells are cells which can differentiate into only one blood cell lineage.

Hematopoietic progenitor cells are a group of cells which covers both pluripotent hematopoietic progenitor cells and unipotent hematopoietic progenitor cells. For example, the hematopoietic progenitor cells in the present invention may be granulocyte-macrophage colony forming cells (CFU-GM), eosinophil colony forming cells (EO-CFC), erythroid burst forming cells (BFU-E) as erythroid progenitor cells, megakaryocyte colony forming cells (CFU-MEG), megakaryocyte progenitor cells, megakaryoblasts, promegakaryocytes, megakaryocyte/erythroid progenitor cells (MEP cells) or myeloid stem cells (mixed colony forming cells, CFU-GEMM). Among them, hematopoietic progenitor cells which differentiate into megakaryocytes and platelets are megakaryocyte colony forming cells (CFU-MEG), megakaryocyte progenitor cells, megakaryoblasts, promegakaryocytes, megakaryocyte/erythroid progenitor cells (MEP cells) and myeloid progentor cells (mixed colony forming cells, CFU-GEMM).

Megakaryocytes are differentiated cells which develop through myeloid progenitor cells, MEP cells, megakaryocyte progenitor cells, megakaryoblasts and promegakaryocytes with an increase in cell size during the cytoplasmic maturation events such as polyploidization, development of the demarcation membrane system and granulation and have the potential to produce platelets through formation of proplatelet processes.

Platelets are anucleate cells derived from megakaryocytes and play an important role in blood coagulation.

CD41a⁺ cells are means expressing CD (cluster of differentiation) 41a antigen on the cell surface. Likewise, CD42b⁺ cells are means expressing CD 42b antigen on the cell surface. These antigens are markers for megakaryocytes and platelets. Populations of CD41a⁺ and CD42b⁺ cells are enriched with megakaryocytes and platelets.

In the present invention, differentiation of hematopoietic progenitor cells means conversion of hematopoietic progenitor cells to mature blood cells having specific functions such as erythrocytes, leukocytes, megakaryocytes and platelets.

The specific compounds to be used in the present invention act on hematopoietic progenitor cells derived from pluripotent stem cells and have such an activity that they induce megakaryopoiesis and thrombopoiesis from such hematopoietic progenitor cells cultured ex vivo in the presence of a specific compound. Even when hematopoietic progenitor cells cannot produce megakaryocytes and platelets efficiently, use of a specific compound makes it possible to produce megakaryocytes and platelets efficiently by culturing hematopoietic progenitor cells derived from pluripotent stem cells ex vivo. Specifically speaking, it is possible to produce megakaryocytes and platelets by culturing hematopoietic progenitor cells in a medium containing a specific compound. It is also possible to produce megakaryocytes and platelets more efficiently by further adding various cytokines or growth factors, by coculturing them with feeder cells or by further adding other low-molecular-weight compounds which act on hematopoietic progenitor cells.

In the present invention, any pluripotent stem cells may be used as long as they have both pluripotency an self-renewal ability and can differentiate into platelets. The pluripotent stem cells may, for example, be ES cells, iPS cells, embryonic germ cells (EG cells), Muse cells or the like, and more preferably ES cells or iPS cells. Examples of transcription factor genes known to be necessary for imparting pluripotency in establishment of iPS cells include Nanog, Oct3/4, Sox2, Klf4, c-Myc and Lin28. iPS can be established by introducing the combination of Oct3/4, Sox2, Klf4 and c-Myc, the combination of Oct3/4, Sox2, Nanog, and Lin28 or the combination of Oct3/4, Sox2 and Klf4 selected from these genes into somatic cells such as fibroblasts. The iPS cells to be used in the present invention may be established by any methods, and in addidtion to those established by introduction of the above-mentioned genes, those established by introduction of genes other than those mentioned above or those established by using a protein or a low-molecular-weight compound may be used.

For culturing and subculturing pluripotent stem cells, a medium usually used to maintain pluripotency may be used. For example, Iscove's Modified Dulbecco's medium (IMDM), Dulbecco's Modified Eagles's Medium (DMEM), F-12 medium, X-VIVO 10 (Lonza), X-VIVO 15 (Lonza), mTeSR (Stemcell Technologies), TeSR2 (Stemcell Technologies), StemProhESC SFM (Invitrogen) and the like may be mentioned. The culture medium may be supplemented with proteins such as basic fibroblast growth factor (bFGF), insulin and transforming growth factor β(TGF-β), serum, KnockOut SR (Invitrogen), amino acids such as glutamine or 2-mercaptoethanol, and the culture vessel may be coated with an extracellular matrix such as laminins-1 to −12, collagen, fibronectin, vitronectin, Matrigel (Becton, Dickinson and Compnay) or Geltrex (Invitrogen). Pluripotent stem cells may be co-cultured with feeder cells. Any feeder cells that contribute to proliferation and maintenance of pluripotent cells may be used, and for example, C3H10T1/2 cell line, OP9 cells, NIH3T3 cells, ST2 cells, PA6 cells, preferably mouse embryonic fibroblast cells (MEF cells) or SL10 cells may be used. It is preferred to suppress growth of feeder cells, for example, by treatment with mitomycin C or irradiation before use.

Pluripotent stem cells are cultured usually at a temperature of from 25 to 39° C., preferably 33 to 39° C., in the atmosphere having a CO₂ concentration of from 4 to 10 vol %, preferably from 4 to 6 vol %.

The source of hematopoietic progenitor cells to be used in the present invention may be an embryoid body obtained by culturing iPS cells or ES cells under conditions suitable to induce differentiation of hematopoietic cells or a sac-like structure, preferably a sac-like structure. An “embryoid body” is an aggregate of cells having a cystic structure obtained in suspension culture of iPS cells or ES cells in the absence of factors for maintaining them in the undifferentiated state and feeder cells (see: Blood, 2003, 102, 906-915). A “sac-like structure” is an iPS or ES cell-derived three-dimensional saccular structure (having a cavity inside) formed of a population of endothelial cells or the like and containing hematopoietic progenitor cells inside. For the details of sac-like structures, see TAKAYAMA et al., BLOOD 2008, 111: 5298-5306.

For preparation of a sac-like structure (hereinafter referred to also as an iPS-sac or ES-sac), suitable culture conditions may be selected, and the suitable culture conditions vary depending on the organism as the source of the iPS cells or ES cells to be used. For example, for human iPS cells or ES cells, IMDM containing fetal bovine serum (FBS) in a final concentration of 15%, optionally supplemented with insulin, transferrin, lactoferrin, cholesterol, ethanolamine, sodium selenite, a-monothioglycerol, 2-mercaptoethanol, bovine serum albumin, sodium pyruvate, ascorbic acid, polyethylene glycol, various vitamins, various amino acids and various antibiotics, may be used as the culture medium. As factors which induce human iPS cells or ES cells to form a sac-like structure, vascular endothelial growth factor (VEGF) and placental growth factor (PGF) may, for example, be mentioned, and VEGF is preferred. VEGF may be added at a concentration of about 10 ng/mL to 100 ng/mL, preferably at a concentration of about 20 ng/mL. A human iPS or ES cell culture may be incubated, for example, in 5% CO₂ at 36 to 38° C., preferably at 37° C., though the incubation conditions differ depending on the human iPS or ES cells to be used. Further, it is possible to produce a sac-like structure more efficiently from human iPS or ES cells by co-culture with feeder cells. Any feeder cells that contribute to induction of differentiation of pluripotent stem cells into hematopoietic progenitor cells may be used, and for example, mouse embryonic fibroblast cells (MEF cells) or SL10 cells, preferably, C3H10T1/2 cell line, OP9 cell line, ST2 cells, NIH3T3 cells, PA6 cells or M15 cells, more preferably C3H10T1/2 cell line or OP9 cells may be used. It is preferred to suppress growth of feeder cells, for example, by treatment with mitomycin C or irradiation before use. The incubation time until formation of a sac-like structure differs depending on the human iPS or ES cells used, and, for example, the presence of a sac-like structure can be confirmed 14 to 17 days after inoculation on feeder cells.

The sac-like structure thus formed has a cystic structure demarcated by septa of cells positive for a mesodermal cell marker Flk1 (fetal liver kinase 1), CD1, CD34 or UEA-1 lectin (Ulex europaeus.agglutinin-1). The inside of the sac-like structure is rich in hematopoietic progenitor cells. Before inducing hematopoietic progenitor cells to differentiate into various blood cells, it is necessary to separate hematopoietic progenitor cells from the cells from the septal cells. The separation may be attained by physical means. For example, the septal cells can be separated from the hematopoietic progenitor cells by breaking a sac-like structure with a pipette or a syringe and then passing the cells through a sterilized sieve-like tool (such as a cell strainer).

In the present invention, the hematopoietic progenitor cells isolated from a suc-like structure or the like as mentioned above are differentiated into megakaryocytes and/or platelets. Differentiation of hematopoietic progenitor cells into platelets means differentiation of hematopoietic progenitor cells into megakaryocytes and production of platelets from the megakaryocytes. Specifically speaking, hematopoietic progenitor cells derived from pluripotent stem cells are cultured under conditions suitable for induction of differentiation of megakaryocytes and/or platelets. To differentiate hematopoietic progenitor cells into megakaryocytes and/or platelets, ordinary culture media used for hematopoietic cell culture, such as Iscove's Modified Dulbecco's medium (IMDM), Dulbecco's Modified Eagles's Medium (DMEM), F-12 medium, X-VIVO 10 (Lonza), X-VIVO 15 (Lonza), McCoy's 5A medium, Eagle's MEM, αMEM, RPMI1640, StemPro34 (Invitrogen), StemSpan H3000 (Stemcell Technologies), StemSpanSFEM(Stemcell Technologies), Stemlinell(Sigma-Aldrich) or QBSF-60(Quality Biological), may be used. As supplements to the media, bovine fetal serum, human serum, horse serum, insulin, transferring, lactoferrin, cholesterol, ethanolamine, sodium selenite, a-monothioglycerol, 2-mercaptoethanol, bovine serum albumin, sodium pyruvate, ascorbic acid, polyethylene glycol, various vitamins, various amino acids, various antibiotics, agar, agarose, collagen, methylcellulose and the like may be mentioned.

“Culturing in the presence of a specific compound” means culturing in a medium containing a specific compound of the present invention, for example, in a medium containing a specific compound only or a medium containing a specific compound together with other differentiation inducing factors. As the other differentiation inducing factors, interleukin-1α (IL-1α), IL-3, IL-4, IL-5, IL-6, IL-9, IL-11, erythropoietin (EPO), granulocyte-macrophage colony-stimulating factor (GM-CSF), stem cell factor (SCF), granulocyte colony-stimulating factor (G-CSF), flk2/flt3 ligand (FL) or Heparin, or a combination of two or more of them may be mentioned. For example, differentiation into megakaryocytes and platelets can be induced in a culture containing a specific compound of the present invention (from 1 ng/mL to 1000 ng/mL, preferably from 10 ng/mL to 200 ng/mL, more preferably from 20 ng/mL to 100 ng/mL), optionally supplemented with SCF (from 10 to 200 ng/mL, preferably about 50 ng/mL) and Heparin (from 10 to 100 U/mL, preferably about 25 U/mL), within about 7 to 15 days. A specific compound of the present invention may be added directly to the culture medium, or after dissolved in an appropriate solvent before use. Examples of the appropriate solvent include dimethyl sulfoxide (DMSO) and various alcohols, but it is not restricted thereto. A specific compound may be immobilized on the surface of a culture plate or a carrier.

A specific compound may be provided or stored in any forms, for example, in a solid form as a tablet, a pill, a capsule or a granule, in a liquid form as a solution or suspension in an appropriate solvent or resolvent, in the form bound to a plate or carrier. When it is formulated into such a form, additives such as a preservative like p-hydroxybenzoates; an excipient like lactose, glucose, sucrose and mannitol; a lubricant like magnesium stearate and talc; a binder like polyvinyl alcohol, hydroxypropylcellulose and gelatin, a surfactant like fatty acid esters, a plasticizer like glycerin may be added. The additives are not restricted to those mentioned above and a person skilled in the art can use any additives of choice.

The culture medium may be supplemented with one or more chemical substances effective in differentiation of hematopoietic progenitor cells into platelets (see: Schweinfurth et al., Platelets, 21: 648-657 2010, Lordier et al., Blood, 112: 3164-3174 2009). Examples of them include retinoic acid receptor ligands such as all-trans-retinoic acid, histone deacetylase inhibitors such as valproic acid, trichostatin A, SAHA (suberoylanilide hydroxamic acid) and APHA (aroyl-pyrrolyl-hydroxyamide), ROCK (Rho-associated coiled-coil forming kinase/Rho-binding kinase) inhibitors such as (R)-(+)-trans-N-(4-pyridyl)-4-(1-aminoethyl)-cyclohexanecarboxamide2HCl.H2O (Y27632), myosin heavy chain II ATPase such as blebbistatin, myosin light chain kinase inhibitors such as ML7 and prostaglandin E2 but are not restricted thereto. Before treating cells with these compounds, a person skilled in the art may determine the optimum concentration of these chemical substances by experiment and may choose appropriate treating time and method. For example, in the case of blebbistatin as a myosin heavy chain II ATPase inhibitor, from 0.3 to 15 μg/mL, or from 1 to 10 μ/mL of blabbistatin may be added, and the incubation time is, preferably, for example, about from 3 to 10 days, particularly, about from 4 to 7 days. Alternatively, Y-27632 as a ROCK inhibitor may be used at 5 to 15 μM, or 8 to 12 μM, preferably about 10 μM, and valproic acid as a HDAC inhibitor may be used at 0.1 to 1 mM, or 0.2 to 0.7 mM, preferably about 0.5 mM. The treatment time for Y-27632 and valproic acid is about from 3 to 21 days, preferably about from 7 to 14 days.

Hematopoietic stem cells and/or hematopoietic progenitor cells are cultured usually at a temperature of from 25 to 39° C., preferably from 33 to 39° C., in the atmosphere having a CO₂ concentration of from 4 to 10 vol %, preferably from 4 to 6 vol %. Hematopoietic progenitor cells may be co-cultured with feeder cells for induction of megakaryocytes and platelets. Any feeder cells that contribute to contribute to induction of differentiation of hematopoietic progenitor cells into megakaryocytes or platelets may be used, and for example, mouse embryonic fibroblast cells (MEF cells) or SL10 cells, preferably, C3H10T1/2 cell line, OP9 cell line, ST2 cells, NIH3T3 cells, PA6 cells or M15 cells, more preferably C3H10T1/2 cell line or OP9 cells may be used. It is preferred to suppress growth of feeder cells, for example, by treatment with mitomycin C or irradiation before use.

Hematopoietic progenitor cells can be cultured in a culture vessel generally used for animal cell culture such as a Petri dish, a flask, a plastic bag, a Teflon (registered trademark) bag, optionally after preliminary coating with an extracellular matrix or a cell adhesion molecule. The material for such a coating may be collagens I to XIX, fibronectin, vitronectin, laminins 1 to 12, nitogen, tenascin, thrombospondin, von Willebrand factor, osteoponin, fibrinogen, various elastins, various proteoglycans, various cadherins, desmocolin, desmoglein, various integrins, E-selectin, P-selectin, L-selectin, immunoglobulin superfamily, Matrigel, poly-D-lysine, poly-L-lysine, chitin, chitosan, Sepharose, alginic acid gel, hydrogel or a fragment thereof. Such a coating material may be a recombinant material having an artificially modified amino acid sequence. Hematopoietic progenitor cells may be cultured by using a bioreactor which can mechanically control the medium composition, pH and the like and obtain high density culture (Schwartz R M, Proc. Natl. Acad. Sci. U.S.A., 88:6760, 1991; Koller M R, Bone Marrow Transplant, 21:653, 1998; Koller, M R, Blood, 82: 378, 1993; Astori G, Bone Marrow Transplant, 35: 1101, 2005).

When megakaryocytes and platelets are produced from human iPS cells or ES cells, since the efficiency of sac-like structure production varies clone to clone, preliminary choice of iPS or ES cell clones which produce a sac-like structure efficiently makes it possible to produce a large number of megakaryocytes and platelets more efficiently from the sac-like structures produced by the selected iPS or ES cell clones. As the iPS or ES cell clones producing a sac-like structure efficiently, clones forming at least 10, preferably at least 15 sac-like structures per 1×10⁵ cells may be chosen.

In addition, introduction of an oncogene or the like increases the proliferative capability of hematopoietic progenitor cells obtained from pluripotent stem cells (see: WO/2011/034073). The oncogene may, for example, be a MYC family gene (such as c-Myc, n-myc or 1-myc gene), a SRC family gene, a RAS family gene, a g RAF family gene, c-kit gene, AbI gene or the like, preferably a gene of the Myc family more preferably c-Myc gene. The senescence of cells resulting from oncogene introduction can be prevented by simultaneous introduction of a polycomb gene or the like. The polycomb gene may, for example, be Bmi1 gene, Mel18 gene, Ring1a/b gene, Phc1/2/3, Cbx2/4/6/7/8 gene, Eed gene, Ezh2 gene or Suz12 gene, preferably Bmi1 gene. Death of cells can be prevented by introduction of an apoptosis suppressor gene, such as BCL2 (B-cell lymphoma 2) gene or BCLXL (B-cell lymphoma-extra large) gene in the BCL2 family or Survivin, MCL1(myeloid cell leukemia1), preferably BCL2 gene or BCLXL gene. Suppression of expression of the tumor suppressor gene p53 is effective for inducing hemeatopoirtic progenitor cells to differentiate into megakaryocytes (see: Fuhrken et al., J. Biol. Chem., 283: 15589-15600 2008). Examples of tumor suppressor genes include p53 gene, p16 gene, p73 gene, Rb gene, BRCA1(breast cancer susceptibility gene 1) gene, BRCA2 gene and WT1 gene, and p53 gene is preferred. In addition, RNA genes which promote thrombopoiesis such as antisense RNAs, small interfering (si) RNAs, short hairpin (sh) RNAs, decoy RNA, ribozymes are also effective as target genes. These genes and RNAs include those having publicly known nucleotide sequences and their homologues obtained by homologous modification of these known sequences by conventional techniques. Hereinafter, genes introduced into hematopoietic progenitor cells for enhancement of their proliferative capability are referred to as target genes.

Target genes may be introduced into cells at any stage of differentiation from pluripotent stem cells to megakaryocytes, such as mesodermal cells, hematopoietic stem cells and hematopoietic progenitor cells. For introduction of target genes into these cells, techniques generally used for transfection of animal cells, for example, vector transfection using animal cell vectors of viral origin for gene therapy (see Verma, I. M., Nature, 389: 239, 1997 for vectors for gene therapy) such as retrovirus vectors represented by mouse stem cell virus (MSCV) and Molonry mouse leukemia virus (MmoLV), adenovirus vectors, adeno-associated vectors (AAVs), herpes simplex virus vectors, lentivirus vectors, Sendai virus vectors, the calcium phosphate coprecipitation method, the DEAE-Dextran method, the electroporation method, the liposome method, the lipofection method and the microinjection method, may be used. Among them, those which allow target genes to be integrated into the chromosomal DNA of the cells and expressed constitutively are preferred, and when viruses are used, retrovirus vectors, adeno-associated virus vectors or lentivirus vectors are preferred.

An adeno-associated virus (AAV) vector may be prepared, for example, as follows. First, 293 cells are transfected with a plasmid vector prepared by inserting a therapeutic gene between the ITRs (inverted terminal repeats) at both end of wild-type adeno-associated virus DNA and a helper plasmid for virus protein supplementation and subsequently with adenovirus as a helper virus or with a plasmid expressing an adenovirus helper genes to produce virus particles containing the AAV vector, which are used for transfection of hematopoietic progenitor cells. It is preferred to insert an appropriate promoter, enhancer, insulator or the like upstream of the target gene to regulate expression of the target gene. A marker gene such as a drug resistance gene may be introduced together with a target gene for easy selection of cells transfected with the target gene. The target gene may be a sense gene or an antisense gene.

A target gene may be introduced into cells via a mammalian expression vector or virus vector carrying the target gene functionally ligated downstream of an appropriate promoter so that the introduced target gene is expressed. Promoters such as CMV promoter, EF promoter and SV40 promoter may be used for constitutive expression of a target gene. A target gene may be functionally ligeted downstream of an endogeneous enhancer/promoter which induces gene expression at a certain stage of differentiation, such as glycoprotein Ilb/IIIa enhance/promoter (see: Nat. Biotech 26, 209-211 (2008)) so that expression of the target gene is induced in the course of differentiation into megakaryocytes. Alternatively, an appropriate promoter may be placed under control of an element whose activity is regulated by a trans factor such as a drug-responsive element to provide a regulatory system which induces or suppresses expression of the target gene by addition of a drug or the like. Elements whose activity is controlled by a drug include, for example, tet operator element (see: Proc. Natl. Acad. Sci. USA 89, 5547-5551 (1992)), GAL4-dingin element (see: Proc. Natl. Acad. Sci. USA 91, 8180-8184 (1992)), Lac operator element (Environ. Mol. Mutagen. 28, 447-458 (1996)) and LexA operator element (see: the EMBO journal 7, 3975-3982 (1988)). Introduction of an appropriate gene having a ligand-binding domain, a DNA-binding domain and a transcriptional regulatory domain which activates or represses transcription responsive to a drug such as tetracycline, dexamthasone, tamoxyfen, estradiol, doxycycline or isopropyl-β-thiogalactopyranoside (IPTG) permits regulation of expression of a target gene downstream of the element by a drug. Transcriptional regulation by binding of a dimeric ligand containing a DNA-binding domain and a transcription regulatory domain is also possible, and it is possible to make such a dimer responsive to a certain drug by using the variable domain of an antibody, as disclosed in Japanese Patent Application2009-201504. For regulated expression of a target gene, for example, the GeneSwitch™ system of Invitrogen, the LacSwitch II Inducible Mammalian Expression system of Agilent Technologies and the iDimerize™ system, the Tet-on™ system and the Tet-off™ system of Clontech may be used. Further, for a drug-responsive regulation of the amount of the protein expressed from a target gene, a target gene to be introduced may be fused with the destabilization domain of a mutant FK506 binding protein by using, for example, the ProtoTuner™ system of Clontech. In addition, it is possible to introduce a target gene at an appropriate location of a genome by using the homologous recombination technique (see: Nature 317, 230-234 (1985)). Further, an introduced target gene or an oncogene in a genome can be removed from the genome or repressed, for example, by using the Cre/Lox system or the FLP/frt system disclosed in Mammalian Genome 15, 677-685 (2004) singly or in combination. Further, removal of a target gene may be attained by directly introducing the Cre protein or the FLP protein at a certain stage of differentiation or by introducing a gene encoding such a protein. A target gene preliminarily introduced into cells can be removed at a certain stage of differentiation by expressing the Cre protein or the FLP protein under control of a drug-responsive element, as in the Cre-ER system disclosed in Developmental Biology 244, 305-318 (2002).

Hematopoietic progenitor cells with enhanced proliferative capability and/or differentiative capability by regulation of expression of a target gene by genetic manipulation may be used for production of megakaryocytes and platelets by the method of the present invention.

Further, the present invention covers a kit for producing platelets as one embodiment. The kit contains a medium for cell culture, serum, a specific compound of the present invention, supplements such as growth factors (such as TPO, SCF, Heparin, IL-6 and IL-11), antibiotics and the like. In addition, it may contain an antibody to detect the marker on sac-like structures (such as antibodies against Flk1, CD31, CD34 and UEA-I lectin). The reagents and antibodies and the like in the kit are supplied in any type of vessel in which components effectively sustain their activity over a long period without being adsorbed or denatured by the material of the vessel, such as a sealed glass ampoule containing a buffer gassed with a neutral inert gas such as nitrogen gas and an ampoule of an organic polymer such as glass, polycarbonate and polystyrene, ceramics, a metal or other appropriate materials usually used to retain a reagent or the like.

Since platelets are effective for preventing and alleviating a decrease in platelets due to leukemia, bone marrow transplantation and anticancer treatment, human platets obtained by the method of the present invention can be stably supplied in the form of a platelet preparation. A platelet preparation can be prepared from platelets produced by the method of the present invention by recovering a fraction of a liquid culture rich in platelets released from magakaryocytes (for example, in the case of human platelets, an approximately 22- to 28 day culture of iPS cells or ES cells) and separating platelets from other components by removing megakaryocytes and other blood cells by using a leukocyte reduction filter (available, for example, from TERUMO and Asahi Kasei Medical) or the like, or by precipitating non-platelet components by centrifugation at 100 to 150 g for 10 to 15 minutes. A platelet preparation may contain other components which stabilize platelets in view of the storage instability of platelets. As conditions for platelet stabilization, a method well known to experts in this technical field may be selected. More specifically, platelets obtained (for example, washed platelets derived from human iPS cells or human ES cells) may be converted to a platelet preparation as follows.

ACD-A solution and FFP (fresh frozen plasma; prepared from whole blood obtained by blood donation, which contains all the other blood components other than blood cells such as albumin and a coagulation factor) are mixed at a ratio of 1:10, irradiated with 15-50 Gy radiation and stored with shaking at 20 to 24° C. The ACD-A solution is prepared by dissolving 22 g of sodium citrate/8 g of citric acid/22 g of glucose with water for injection to a total volume of 1 L.

When the above-mentioned method is used, the platelet density is set desirably at 1×10⁹ platelets/mL, for example.

Addition of GM6001(a broad-range hydroxamic acid-based metalloprotease inhibitor) (Calbiochem, La Jolla, Calif., USA) prevents platelet deactivation during cryopreservation and storage at room temperature caused by cleavage of the molecules essential for platelet function such as GPIb-V-1× and GPVI. The present inventors confirmed that inactivation of platelets derived from mouse ES cells can be prevented by this method. For the mechanisms of inactivation of human platelets by this method, see Bergmeier, W et al., Cir Res 95: 677-683, 2004 and Gardiner, E E et al., J Thrombosis and Haemostasis, 5:1530-1537, 2007.

For a container of a preparation containing platelets, materials which activate platelets such as glass are preferably avoided.

Platelets produced by the method of the present invention may be used for treatment of diseases accompanied by a decrease in platelets and effective for treatment of various diseases.

As specific examples, lysosomal storage disease such as Gaucher's disease and mucopolysaccharidosis, adrenoleukodystrophy, various kinds of cancers and tumors, especially blood cancers such as acute or chronic leukemia, Fanconi syndrome, aplastic anemia, granulocytopenia, lymphopenia, thrombocytopenia, idiopathic thrombocytopenic purpura, thrombotic thrombocytopenic purpura, Kasabach-Merritt syndrome, malignant lymphoma, Hodgkin's disease, multiple myeloma, chronic hepatopathy, renal failure, massive blood transfusion of bank blood or during operation, hepatitis B, hepatitis C, severe infections, systemic lupus erythematodes, articular rheumatism, xerodermosteosis, systemic sclerosis, polymyositis, dermatomyositis, mixed connective tissue disease, polyarteritis nodosa, Hashimoto's disease, Basedow's disease, myasthenia gravis, insulin dependent diabetes mellitus, autoimmune hemolytic anemia, snake bite, hemolytic uremic syndrome, hypersplenism, bleeding, Bernard-Soulier syndrome, Glanzmann's thrombasthenia, uremia, myelodysplastic syndrome, polycythemia rubra vera, erythremia, myeloproliferative disease, and the like may be mentioned. Now, the specific compound to be used in the present invention will be described in terms of the definitions of terms used for it and its best mode.

In the compound to be used in the present invention, “n” denotes normal, “i” denotes iso, “s” denotes secondary, “t” denotes tertiary, “c” denotes cyclo, “o” denotes ortho, “m” denotes meta, and “p” denotes para.

First, the terms in the respective substituents R¹ to R²⁰ and V¹ to V⁴ will be explained.

As a halogen atom, a fluorine atom, a chlorine atom, a bromine atom or an iodine atom may be mentioned.

A C₁₋₃ alkyl group may be linear, branched or a O₃ cycloalkyl group, and methyl, ethyl, n-propyl, i-propyl and c-propyl and the like may be mentioned.

A C₁₋₆ alkyl group may be linear, branched or a C₃₋₆ cycloalkyl group, and as specific examples, in addition to those mentioned above, n-butyl, i-butyl, s-butyl, t-butyl, c-butyl, 1-methyl-c-propyl, 2-methyl-c-propyl, n-pentyl, 1-methyl-n-butyl, 2-methyl-n-butyl, 3-methyl-n-butyl, 1,1-dimethyl-n-propyl, 1,2-dimethyl-n-propyl, 2,2-dimethyl-n-propyl, 1-ethyl-n-propyl, c-pentyl, 1-methyl-c-butyl, 2-methyl-c-butyl, 3-methyl-c-butyl, 1,2-dimethyl-c-propyl, 2,3-dimethyl-c-propyl, 1-ethyl-c-propyl, 2-ethyl-c-propyl, n-hexyl, 1-methyl-n-pentyl, 2-methyl-n-pentyl, 3-methyl-n-pentyl, 4-methyl-n-pentyl, 1,1-dimethyl-n-butyl, 1,2-dimethyl-n-butyl, 1,3-dimethyl-n-butyl, 2,2-dimethyl-n-butyl, 2,3-dimethyl-n-butyl, 3,3-dimethyl-n-butyl, 1-ethyl-n-butyl, 2-ethyl-n-butyl, 1,1,2-trimethyl-n-propyl, 1,2,2-trimethyl-n-propyl, 1-ethyl-1-methyl-n-propyl, 1-ethyl-2-methyl-n-propyl, c-hexyl, 1-methyl-c-pentyl, 2-methyl-c-pentyl, 3-methyl-c-pentyl, 1-ethyl-c-butyl, 2-ethyl-c-butyl, 3-ethyl-c-butyl, 1,2-dimethyl-c-butyl, 1,3-dimethyl-c-butyl, 2,2-dimethyl-c-butyl, 2,3-dimethyl-c-butyl, 2,4-dimethyl-c-butyl, 3,3-dimethyl-c-butyl, 1-n-propyl-c-propyl, 2-n-propyl-c-propyl, 1-i-propyl-c-propyl, 2-i-propyl-c-propyl, 1,2,2-trimethyl-c-propyl, 1,2,3-trimethyl-c-propyl, 2,2,3-trimethyl-c-propyl, 1-ethyl-2-methyl-c-propyl, 2-ethyl-1-methyl-c-propyl, 2-ethyl-2-methyl-c-propyl, 2-ethyl-3-methyl-c-propyl and the like may be mentioned.

A C₁₋₁₀ alkyl group may be linear, branched or a C₃₋₁₀ cycloalkyl group, and as specific examples, in addition to those mentioned above, 1-methyl-1-ethyl-n-pentyl, 1-heptyl, 2-heptyl, 1-ethyl-1,2-dimethyl-n-propyl, 1-ethyl-2,2-dimethyl-n-propyl, 1-octyl, 3-octyl, 4-methyl-3-n-heptyl, 6-methyl-2-n-heptyl, 2-propyl-1-n-heptyl, 2,4,4-trimethyl-1-n-pentyl, 1-nonyl, 2-nonyl, 2,6-dimethyl-4-n-heptyl, 3-ethyl-2,2-dimethyl-3-n-pentyl, 3,5,5-trimethyl-1-n-hexyl, 1-decyl, 2-decyl, 4-decyl, 3,7-dimethyl-1-n-octyl, 3,7-dimethyl-3-n-octyl and the like may be mentioned.

A C₂₋₆ alkenyl group may be linear, branched or a C₃₋₆ cycloalkenyl group, and as specific examples, ethenyl, 1-propenyl, 2-propenyl, 1-methyl-1-ethenyl, 1-butenyl, 2-butenyl, 3-butenyl, 2-methyl-1-propenyl, 2-methyl-2-propenyl, 1-ethylethenyl, 1-methyl-1-propenyl, 1-methyl-2-propenyl, 1-pentenyl, 2-pentenyl, 3-pentenyl, 4-pentenyl, 1-n-propylethenyl, 1-methyl-1-butenyl, 1-methyl-2-butenyl, 1-methyl-3-butenyl, 2-ethyl-2-propenyl, 2-methyl-1-butenyl, 2-methyl-2-butenyl, 2-methyl-3-butenyl, 3-methyl-1-butenyl, 3-methyl-2-butenyl, 3-methyl-3-butenyl, 1,1-dimethyl-2-propenyl, 1-i-propylethenyl, 1,2-dimethyl-1-propenyl, 1,2-dimethyl-2-propenyl, 1-c-pentenyl, 2-c-pentenyl, 3-c-pentenyl, 1-hexenyl, 2-hexenyl, 3-hexenyl, 4-hexenyl, 5-hexenyl, 1-methyl-1-pentenyl, 1-methyl-2-pentenyl, 1-methyl-3-pentenyl, 1-methyl-4-pentenyl, 1-n-butylethenyl, 2-methyl-1-pentenyl, 2-methyl-2-pentenyl, 2-methyl-3-pentenyl, 2-methyl-4-pentenyl, 2-n-propyl-2-propenyl, 3-methyl-1-pentenyl, 3-methyl-2-pentenyl, 3-methyl-3-pentenyl, 3-methyl-4-pentenyl, 3-ethyl-3-butenyl, 4-methyl-1-pentenyl, 4-methyl-2-pentenyl, 4-methyl-3-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1-methyl-2-ethyl-2-propenyl, 1-s-butylethenyl, 1,3-dimethyl-1-butenyl, 1,3-dimethyl-2-butenyl, 1,3-dimethyl-3-butenyl, 1-i-butylethenyl, 2,2-dimethyl-3-butenyl, 2,3-dimethyl-1-butenyl, 2,3-dimethyl-2-butenyl, 2,3-dimethyl-3-butenyl, 2-i-propyl-2-propenyl, 3,3-dimethyl-1-butenyl, 1-ethyl-1-butenyl, 1-ethyl-2-butenyl, 1-ethyl-3-butenyl, 1-n-propyl-1-propenyl, 1-n-propyl-2-propenyl, 2-ethyl-1-butenyl, 2-ethyl-2-butenyl, 2-ethyl-3-butenyl, 1,1,2-trimethyl-2-propenyl, 1-t-butylethenyl, 1-methyl-1-ethyl-2-propenyl, 1-ethyl-2-methyl-1-propenyl, 1-ethyl-2-methyl-2-propenyl, 1-i-propyl-1-propenyl, 1-i-propyl-2-propenyl, 1-methyl-2-c-pentenyl, 1-methyl-3-c-pentenyl, 2-methyl-1-c-pentenyl, 2-methyl-2-c-pentenyl, 2-methyl-3-c-pentenyl, 2-methyl-4-c-pentenyl, 2-methyl-5-c-pentenyl, 2-methylene-c-pentyl, 3-methyl-1-c-pentenyl, 3-methyl-2-c-pentenyl, 3-methyl-3-c-pentenyl, 3-methyl-4-c-pentenyl, 3-methyl-5-c-pentenyl, 3-methylene-c-pentyl, 1-c-hexenyl, 2-c-hexenyl, 3-c-hexenyl and the like may be mentioned.

As a C₂₋₆ alkynyl group, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 1-n-propyl-2-propynyl, 2-ethyl-3-butynyl, 1-methyl-1-ethyl-2-propynyl, 1-i-propyl-2-propynyl and the like may be mentioned.

A C₂₋₁₄ aryl group may be a C₆₋₁₄ aryl group containing no hetero atoms as ring constituting atoms, a C₂₋₉ heteroaryl group or a C₂₋₁₄ fused polycyclic group.

As a C₆₋₁₄ aryl group containing no hetero atoms, a phenyl group, a 1-indenyl group, a 2-indenyl group, a 3-indenyl group, a 4-indenyl group, a 5-indenyl group, a 6-indenyl group, a 7-indenyl group, an α-naphthyl group, a β-naphthyl group, an o-biphenylyl group, a m-biphenylyl group, a p-biphenylyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group,

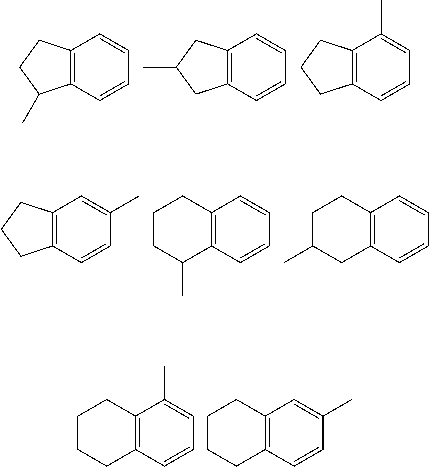

or the like may be mentioned.

A C₂₋₉ heteroaryl group may be a 5 to 7-membered C₂₋₆ heteromonocyclic group or 8 to 10-membered C₅₋₉ fused heterobicyclic group containing from 1 to 3 oxygen atoms, nitrogen atoms or sulfur atoms singly or in combination.

As a 5 to 7-membered C₂₋₆ heteromonocyclic group, a 2-thienyl group, a 3-thienyl group, a 2-furyl group, a 3-furyl group, a 2-pyranyl group, a 3-pyranyl group, a 4-pyranyl group, a 1-pyrrolyl group, a 2-pyrrolyl group, a 3-pyrrolyl group, a 1-imidazolyl group, a 2-imidazolyl group, a 4-imidazolyl group, a 1-pyrazolyl group, a 3-pyrazolyl group, a 4-pyrazolyl group, a 2-thiazolyl group, a 4-thiazolyl group, a 5-thiazolyl group, a 3-isothiazolyl group, a 4-isothiazolyl group, a 5-isothiazolyl group, a 1-1,2,4-triazole group, a 3-1,2,4-triazole group, a 5-1,2,4-triazole group, a 1-1,2,3-triazole group, a 4-1,2,3-triazole group, a 5-1,2,3-triazole group, a 2-oxazolyl group, a 4-oxazolyl group, a 5-oxazolyl group, a 3-isoxazolyl group, a 4-isoxazolyl group, a 5-isoxazolyl group, a 2-pyridyl group, a 3-pyridyl group, a 4-pyridyl group, a 2-pyrazinyl group, a 2-pyrimidinyl group, a 4-pyrimidinyl group, a 5-pyrimidinyl group, a 3-pyridazinyl group, a 4-pyridazinyl group, a 2-1,3,4-oxadiazolyl group, a 2-1,3,4-thiadiazolyl group, a 3-1,2,4-oxadiazolyl group, a 5-1,2,4-oxadiazolyl group, a 3-1,2,4-thiadiazolyl group, a 5-1,2,4-thiadiazolyl group, a 3-1,2,5-oxadiazolyl group, a 3-1,2,5-thiadiazolyl group or the like may be mentioned.

As a 8 to 10-membered C₅₋₉ fused heterocyclic group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 4-isobenzofuranyl group, a 5-isobenzofuranyl group, a 2-benzothienyl group, a 3-benzothienyl group, a 4-benzothienyl group, a 5-benzothienyl group, a 6-benzothienyl group, a 7-benzothienyl group, a 1-isobenzothienyl group, a 4-isobenzothienyl group, a 5-isobenzothienyl group, a 2-chromenyl group, a 3-chromenyl group, a 4-chromenyl group, a 5-chromenyl group, a 6-chromenyl group, a 7-chromenyl group, a 8-chromenyl group, a 1-indolizinyl group, a 2-indolizinyl group, a 3-indolizinyl group, a 5-indolizinyl group, a 6-indolizinyl group, a 7-indolizinyl group, a 8-indolizinyl group, a 1-isoindolyl group, a 2-isoindolyl group, a 4-isoindolyl group, a 5-isoindolyl group, a 1-indolyl group, a 2-indolyl group, a 3-indolyl group, a 4-indolyl group, a 5-indolyl group, a 6-indolyl group, a 7-indolyl group, 1-indazolyl group, a 2-indazolyl group, a 3-indazolyl group, a 4-indazolyl group, a 5-indazolyl group, a 6-indazolyl group, a 7-indazolyl group, a 1-purinyl group, a 2-purinyl group, a 3-purinyl group, a 6-purinyl group, a 7-purinyl group, a 8-purinyl group, a 2-quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 1-phthalazinyl group, a 5-phthalazinyl group, a 6-phthalazinyl group, a 1-2,7-naphthyridinyl group, a 3-2,7-naphthyridinyl group, a 4-2,7-naphthyridinyl group, a 1-2,6-naphthyridinyl group, a 3-2,6-naphthyridinyl group, a 4-2,6-naphthyridinyl group, a 2-1,8-naphthyridinyl group, a 3-1,8-naphthyridinyl group, a 4-1,8-naphthyridinyl group, a 2-1,7-naphthyridinyl group, a 3-1,7-naphthyridinyl group, a 4-1,7-naphthyridinyl group, a 5-1,7-naphthyridinyl group, a 6-1,7-naphthyridinyl group, a 8-1,7-naphthyridinyl group, 2-1,6-naphthyridinyl group, a 3-1,6-naphthyridinyl group, a 4-1,6-naphthyridinyl group, a 5-1,6-naphthyridinyl group, a 7-1,6-naphthyridinyl group, a 8-1,6-naphthyridinyl group, a 2-1,5-naphthyridinyl group, a 3-1,5-naphthyridinyl group, a 4-1,5-naphthyridinyl group, a 6-1,5-naphthyridinyl group, a 7-1,5-naphthyridinyl group, a 8-1,5-naphthyridinyl group, a 2-quinoxalinyl group, a 5-quinoxalinyl group, a 6-quinoxalinyl group, a 2-quinazolinyl group, a 4-quinazolinyl group, a 5-quinazolinyl group, a 6-quinazolinyl group, a 7-quinazolinyl group, a 8-quinazolinyl group, a 3-cinnolinyl group, a 4-cinnolinyl group, a 5-cinnolinyl group, a 6-cinnolinyl group, a 7-cinnolinyl group, a 8-cinnolinyl group, a 2-pteridinyl group, a 4-pteridinyl group, a 6-pteridinyl group, a 7-pteridinyl group or the like may be mentioned.

A C₂₋₉ nitrogen-containing heteroaryl group is a C₂₋₉ heteroaryl group containing one to three nitrogen atoms.

A C₂₋₁₄ fused polycyclic group is a fused bicyclic or fused tricyclic group consisting of a C₆₋₁₄ aryl group containing no hetero atoms and at most 12 carbon atoms as mentioned above or a C₂₋₉ heteroaryl group fused with a C₂₋₉ heterocyclyl group, and:

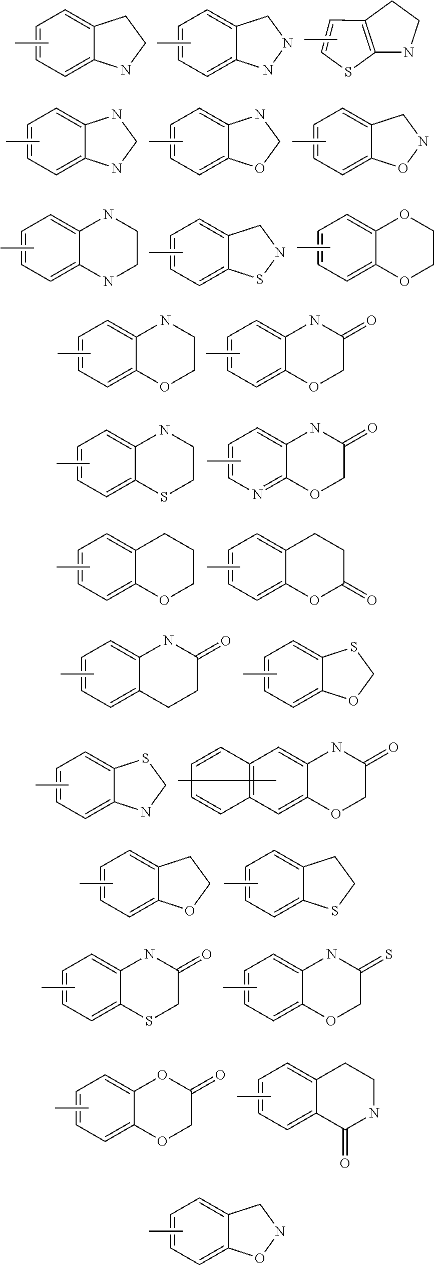

may be mentioned specifically.

A C₁₋₁₀ thioalkyl group may linear, branched or a C₃₋₁₀ cyclothioalkyl group, and as specific examples, methylthio, ethylthio, n-propylthio, i-propylthio, c-propylthio, n-butylthio, i-butylthio, s-butylthio, t-butylthio, c-butylthio, 1-methyl-c-propylthio, 2-methyl-c-propylthio, n-pentylthio, 1-methyl-n-butylthio, 2-methyl-n-butylthio, 3-methyl-n-butylthio, 1,1-dimethyl-n-propylthio, 1,2-dimethyl-n-propylthio, 2,2-dimethyl-n-propylthio, 1-ethyl-n-propylthio, c-pentylthio, 1-methyl-c-butylthio, 2-methyl-c-butylthio, 3-methyl-c-butylthio, 1,2-dimethyl-c-propylthio, 2,3-dimethyl-c-propylthio, 1-ethyl-c-propylthio, 2-ethyl-c-propylthio, n-hexylthio, 1-methyl-n-pentylthio, 2-methyl-n-pentylthio, 3-methyl-n-pentylthio, 4-methyl-n-pentylthio, 1,1-dimethyl-n-butylthio, 1,2-dimethyl-n-butylthio, 1,3-dimethyl-n-butylthio, 2,2-dimethyl-n-butylthio, 2,3-dimethyl-n-butylthio, 3,3-dimethyl-n-butylthio, 1-ethyl-n-butylthio, 2-ethyl-n-butylthio, 1,1,2-trimethyl-n-propylthio, 1,2,2-trimethyl-n-propylthio, 1-ethyl-1-methyl-n-propylthio, 1-ethyl-2-methyl-n-propylthio, c-hexylthio, 1-methyl-c-pentylthio, 2-methyl-c-pentylthio, 3-methyl-c-pentylthio, 1-ethyl-c-butylthio, 2-ethyl-c-butylthio, 3-ethyl-c-butylthio, 1,2-dimethyl-c-butylthio, 1,3-dimethyl-c-butylthio, 2,2-dimethyl-c-butylthio, 2,3-dimethyl-c-butylthio, 2,4-dimethyl-c-butylthio, 3,3-dimethyl-c-butylthio, 1-n-propyl-c-propylthio, 2-n-propyl-c-propylthio, 1-i-propyl-c-propylthio, 2-i-propyl-c-propylthio, 1,2,2-trimethyl-c-propylthio, 1,2,3-trimethyl-c-propylthio, 2,2,3-trimethyl-c-propylthio, 1-ethyl-2-methyl-c-propylthio, 2-ethyl-1-methyl-c-propylthio, 2-ethyl-2-methyl-c-propylthio, 2-ethyl-3-methyl-c-propylthio, 1-methyl-1-ethyl-n-pentylthio, 1-heptylthio, 2-heptylthio, 1-ethyl-1,2-dimethyl-n-propylthio, 1-ethyl-2,2-dimethyl-n-propylthio, 1-octylthio, 3-octylthio, 4-methyl-3-n-heptylthio, 6-methyl-2-n-heptylthio, 2-propyl-1-n-heptylthio, 2,4,4-trimethyl-1-n-pentylthio, 1-nonylthio, 2-nonylthio, 2,6-dimethyl-4-n-heptylthio, 3-ethyl-2,2-dimethyl-3-n-pentylthio, 3,5,5-trimethyl-1-n-hexylthio, 1-decylthio, 2-decylthio, 4-decylthio, 3,7-dimethyl-1-n-octylthio, 3,7-dimethyl-3-n-octylthio or the like may be mentioned.

A C₁₋₃ alkylsulfonyl group may be linear, branched or a C₃ cycloalkylsulfonyl group, and as specific examples, methylsulfonyl, ethylsulfonyl, n-propylsulfonyl, i-propylsulfonyl, c-propylsulfonyl or the like may be mentioned.

A C₁₋₁₀ alkylsulfonyl group may be linear, branched or a C₃₋₁₀ cycloalkylsulfonyl group, and as specific examples, in addition to those mentioned above, n-butylsulfonyl, i-butylsulfonyl, s-butylsulfonyl, t-butylsulfonyl, c-butylsulfonyl, 1-methyl-c-propylsulfonyl, 2-methyl-c-propylsulfonyl, n-pentylsulfonyl, 1-methyl-n-butylsulfonyl, 2-methyl-n-butylsulfonyl, 3-methyl-n-butylsulfonyl, 1,1-dimethyl-n-propylsulfonyl, 1,2-dimethyl-n-propylsulfonyl, 2,2-dimethyl-n-propylsulfonyl, 1-ethyl-n-propylsulfonyl, c-pentylsulfonyl, 1-methyl-c-butylsulfonyl, 2-methyl-c-butylsulfonyl, 3-methyl-c-butylsulfonyl, 1,2-dimethyl-c-propylsulfonyl, 2,3-dimethyl-c-propylsulfonyl, 1-ethyl-c-propylsulfonyl, 2-ethyl-c-propylsulfonyl, n-hexylsulfonyl, 1-methyl-n-pentylsulfonyl, 2-methyl-n-pentylsulfonyl, 3-methyl-n-pentylsulfonyl, 4-methyl-n-pentylsulfonyl, 1,1-dimethyl-n-butylsulfonyl, 1,2-dimethyl-n-butylsulfonyl, 1,3-dimethyl-n-butylsulfonyl, 2,2-dimethyl-n-butylsulfonyl, 2,3-dimethyl-n-butylsulfonyl, 3,3-dimethyl-n-butylsulfonyl, 1-ethyl-n-butylsulfonyl, 2-ethyl-n-butylsulfonyl, 1,1,2-trimethyl-n-propylsulfonyl, 1,2,2-trimethyl-n-propylsulfonyl, 1-ethyl-1-methyl-n-propylsulfonyl, 1-ethyl-2-methyl-n-propylsulfonyl, c-hexylsulfonyl, 1-methyl-c-pentylsulfonyl, 2-methyl-c-pentylsulfonyl, 3-methyl-c-pentylsulfonyl, 1-ethyl-c-butylsulfonyl, 2-ethyl-c-butylsulfonyl, 3-ethyl-c-butylsulfonyl, 1,2-dimethyl-c-butylsulfonyl, 1,3-dimethyl-c-butylsulfonyl, 2,2-dimethyl-c-butylsulfonyl, 2,3-dimethyl-c-butylsulfonyl, 2,4-dimethyl-c-butylsulfonyl, 3,3-dimethyl-c-butylsulfonyl, 1-n-propyl-c-propylsulfonyl, 2-n-propyl-c-propylsulfonyl, 1-i-propyl-c-propylsulfonyl, 2-i-propyl-c-propylsulfonyl, 1,2,2-trimethyl-c-propylsulfonyl, 1,2,3-trimethyl-c-propylsulfonyl, 2,2,3-trimethyl-c-propylsulfonyl, 1-ethyl-2-methyl-c-propylsulfonyl, 2-ethyl-1-methyl-c-propylsulfonyl, 2-ethyl-2-methyl-c-propylsulfonyl, 2-ethyl-3-methyl-c-propylsulfonyl, 1-methyl-1-ethyl-n-pentylsulfonyl, 1-heptylsulfonyl, 2-heptylsulfonyl, 1-ethyl-1,2-dimethyl-n-propylsulfonyl, 1-ethyl-2,2-dimethyl-n-propylsulfonyl, 1-octylsulfonyl, 3-octylsulfonyl, 4-methyl-3-n-heptylsulfonyl, 6-methyl-2-n-heptylsulfonyl, 2-propyl-1-n-heptylsulfonyl, 2,4,4-trimethyl-1-n-pentylsulfonyl, 1-nonylsulfonyl, 2-nonylsulfonyl, 2,6-dimethyl-4-n-heptylsulfonyl, 3-ethyl-2,2-dimethyl-3-n-pentylsulfonyl, 3,5,5-trimethyl-1-n-hexylsulfonyl, 1-decylsulfonyl, 2-decylsulfonyl, 4-decylsulfonyl, 3,7-dimethyl-1-n-octylsulfonyl, 3,7-dimethyl-3-n-octylsulfonyl or the like may be mentioned.

A C₁₋₁₀ alkylsulfonylamino group may be linear, branched or a C₃₋₁₀ cycloalkylsulfonylamino group, and as specific examples, methylsulfonylamino, ethylsulfonylamino, n-propylsulfonylamino, i-propylsulfonylamino, c-propylsulfonylamino, n-butylsulfonylamino, i-butylsulfonylamino, s-butylsulfonylamino, t-butylsulfonylamino, c-butylsulfonylamino, 1-methyl-c-propylsulfonylamino, 2-methyl-c-propylsulfonylamino, n-pentylsulfonylamino, 1-methyl-n-butylsulfonylamino, 2-methyl-n-butylsulfonylamino, 3-methyl-n-butylsulfonylannino, 1,1-dimethyl-n-propylsulfonylamino, 1,2-dimethyl-n-propylsulfonylamino, 2,2-dimethyl-n-propylsulfonylamino, 1-ethyl-n-propylsulfonylamino, c-pentylsulfonylamino, 1-methyl-c-butylsulfonylamino, 2-methyl-c-butylsulfonylamino, 3-methyl-c-butylsulfonylamino, 1,2-dimethyl-c-propylsulfonylamino, 2,3-dimethyl-c-propylsulfonylamino, 1-ethyl-c-propylsulfonylamino, 2-ethyl-c-propylsulfonylamino, n-hexylsulfonylamino, 1-methyl-n-pentylsulfonylamino, 2-methyl-n-pentylsulfonylamino, 3-methyl-n-pentylsulfonylamino, 4-methyl-n-pentylsulfonylamino, 1,1-dimethyl-n-butylsulfonylamino, 1,2-dimethyl-n-butylsulfonylamino, 1,3-dimethyl-n-butylsulfonylamino, 2,2-dimethyl-n-butylsulfonylamino, 2,3-dimethyl-n-butylsulfonylamino, 3,3-dimethyl-n-butylsulfonylamino, 1-ethyl-n-butylsulfonylamino, 2-ethyl-n-butylsulfonylamino, 1,1,2-trimethyl-n-propylsulfonylamino, 1,2,2-trimethyl-n-propylsulfonylamino, 1-ethyl-1-methyl-n-propylsulfonylamino, 1-ethyl-2-methyl-n-propylsulfonylamino, c-hexylsulfonylamino, 1-methyl-c-pentylsulfonylamino, 2-methyl-c-pentylsulfonylamino, 3-methyl-c-pentylsulfonylamino, 1-ethyl-c-butylsulfonylamino, 2-ethyl-c-butylsulfonylamino, 3-ethyl-c-butylsulfonylamino, 1,2-dimethyl-c-butylsulfonylamino, 1,3-dimethyl-c-butylsulfonylamino, 2,2-dimethyl-c-butylsulfonylamino, 2,3-dimethyl-c-butylsulfonylamino, 2,4-dimethyl-c-butylsulfonylamino, 3,3-dimethyl-c-butylsulfonylamino, 1-n-propyl-c-propylsulfonylamino, 2-n-propyl-c-propylsulfonylamino, 1-i-propyl-c-propylsulfonylamino, 2-i-propyl-c-propylsulfonylamino, 1,2,2-trimethyl-c-propylsulfonylamino, 1,2,3-trimethyl-c-propylsulfonylamino, 2,2,3-trimethyl-c-propylsulfonylamino, 1-ethyl-2-methyl-c-propylsulfonylamino, 2-ethyl-1-methyl-c-propylsulfonylamino, 2-ethyl-2-methyl-c-propylsulfonylamino, 2-ethyl-3-methyl-c-propylsulfonylamino, 1-methyl-1-ethyl-n-pentylsulfonylamino, 1-heptylsulfonylamino, 2-heptylsulfonylamino, 1-ethyl-1,2-dimethyl-n-propylsulfonylamino, 1-ethyl-2,2-dimethyl-n-propylsulfonylamino, 1-octylsulfonylamino, 3-octylsulfonylamino, 4-methyl-3-n-heptylsulfonylamino, 6-methyl-2-n-heptylsulfonylamino, 2-propyl-1-n-heptylsulfonylamino, 2,4,4-trimethyl-1-n-pentylsulfonylamino, 1-nonylsulfonylamino, 2-nonylsulfonylamino, 2,6-dimethyl-4-n-heptylsulfonylamino, 3-ethyl-2,2-dimethyl-3-n-pentylsulfonylamino, 3,5,5-trimethyl-1-n-hexylsulfonylamino, 1-decylsulfonylamino, 2-decylsulfonylamino, 4-decylsulfonylamino, 3,7-dimethyl-1-n-octylsulfonylamino, 3,7-dimethyl-3-n-octylsulfonylamino, c-heptylsulfonylamino, c-octylsulfonylamino, 1-methyl-c-hexylsulfonylamino, 2-methyl-c-hexylsulfonylamino, 3-methyl-c-hexylsulfonylamino, 1,2-dimethyl-c-hexylsulfonylamino, 1-ethyl-c-hexylsulfonylamino, 1-methyl-c-pentylsulfonylamino, 2-methyl-c-pentylsulfonylamino, 3-methyl-c-pentylsulfonylamino or the like may be mentioned.

A C₁₋₃ alkoxy group may be linear, branched or a C₃ cycloalkoxy group, and as specific examples, methoxy, ethoxy, n-propoxy, i-propoxy, c-propoxy or the like may be mentioned.

A C₁₋₆ alkoxy group may be linear, branched or a C₃₋₆ cycloalkoxy group, and as specific examples, in addition to those mentioned above, n-butoxy, i-butoxy, s-butoxy, t-butoxy, c-butoxy, 1-methyl-c-propoxy, 2-methyl-c-propoxy, n-pentyloxy, 1-methyl-n-butoxy, 2-methyl-n-butoxy, 3-methyl-n-butoxy, 1,1-dimethyl-n-propoxy, 1,2-dimethyl-n-propoxy, 2,2-dimethyl-n-propoxy, 1-ethyl-n-propoxy, c-pentyloxy, 1-methyl-c-butoxy, 2-methyl-c-butoxy, 3-methyl-c-butoxy, 1,2-dimethyl-c-propoxy, 2,3-dimethyl-c-propoxy, 1-ethyl-c-propoxy, 2-ethyl-c-propoxy, n-hexyloxy, 1-methyl-n-pentyloxy, 2-methyl-n-pentyloxy, 3-methyl-n-pentyloxy, 4-methyl-n-pentyloxy, 1,1-dimethyl-n-butoxy, 1,2-dimethyl-n-butoxy, 1,3-dimethyl-n-butoxy, 2,2-dimethyl-n-butoxy, 2,3-dimethyl-n-butoxy, 3,3-dimethyl-n-butoxy, 1-ethyl-n-butoxy, 2-ethyl-n-butoxy, 1,1,2-trimethyl-n-propoxy, 1,2,2-trimethyl-n-propoxy, 1-ethyl-1-methyl-n-propoxy, 1-ethyl-2-methyl-n-propoxy, c-hexyloxy, 1-methyl-c-pentyloxy, 2-methyl-c-pentyloxy, 3-methyl-c-pentyloxy, 1-ethyl-c-butoxy, 2-ethyl-c-butoxy, 3-ethyl-c-butoxy, 1,2-dimethyl-c-butoxy, 1,3-dimethyl-c-butoxy, 2,2-dimethyl-c-butoxy, 2,3-dimethyl-c-butoxy, 2,4-dimethyl-c-butoxy, 3,3-dimethyl-c-butoxy, 1-n-propyl-c-propoxy, 2-n-propyl-c-propoxy, 1-i-propyl-c-propoxy, 2-i-propyl-c-propoxy, 1,2,2-trimethyl-c-propoxy, 1,2,3-trimethyl-c-propoxy, 2,2,3-trimethyl-c-propoxy, 1-ethyl-2-methyl-c-propoxy, 2-ethyl-1-methyl-c-propoxy, 2-ethyl-2-methyl-c-propoxy, 2-ethyl-3-methyl-c-propoxy or the like may be mentioned.

A C₁₋₁₀ alkoxy group may be linear, branched or a C₃₋₁₀ cycloalkoxy group, and as specific examples, in addition to those mentioned above, 1-methyl-1-ethyl-n-pentyloxy, 1-heptyloxy, 2-heptyloxy, 1-ethyl-1,2-dimethyl-n-propyloxy, 1-ethyl-2,2-dimethyl-n-propyloxy, 1-octyloxy, 3-octyloxy, 4-methyl-3-n-heptyloxy, 6-methyl-2-n-heptyloxy, 2-propyl-1-n-heptyloxy, 2,4,4-trimethyl-1-n-pentyloxy, 1-nonyloxy, 2-nonyloxy, 2,6-dimethyl-4-n-heptyloxy, 3-ethyl-2,2-dimethyl-3-n-pentyloxy, 3,5,5-trimethyl-1-n-hexyloxy, 1-decyloxy, 2-decyloxy, 4-decyloxy, 3,7-dimethyl-1-n-octyloxy, 3,7-dimethyl-3-n-octyloxy or the like may be mentioned.

A C₁₋₁₀ alkoxycarbonyl group may be linear, branched or a C₃₋₁₀ cycloalkoxycarbonyl group, and as specific examples, methoxycarbonyl, ethoxycarbonyl, n-propoxycarbonyl, i-propoxycarbonyl, c-propoxycarbonyl, n-butoxycarbonyl, i-butoxycarbonyl, s-butoxycarbonyl, t-butoxycarbonyl, c-butoxycarbonyl, 1-methyl-c-propoxycarbonyl, 2-methyl-c-propoxycarbonyl, n-pentyloxycarbonyl, 1-methyl-n-butoxycarbonyl, 2-methyl-n-butoxycarbonyl, 3-methyl-n-butoxycarbonyl, 1,1-dimethyl-n-propoxycarbonyl, 1,2-dimethyl-n-propoxycarbonyl, 2,2-dimethyl-n-propoxycarbonyl, 1-ethyl-n-propoxycarbonyl, c-pentyloxycarbonyl, 1-methyl-c-butoxycarbonyl, 2-methyl-c-butoxycarbonyl, 3-methyl-c-butoxycarbonyl, 1,2-dimethyl-c-propoxycarbonyl, 2,3-dimethyl-c-propoxycarbonyl, 1-ethyl-c-propoxycarbonyl, 2-ethyl-c-propoxycarbonyl, n-hexyloxycarbonyl, 1-methyl-n-pentyloxycarbonyl, 2-methyl-n-pentyloxycarbonyl, 3-methyl-n-pentyloxycarbonyl, 4-methyl-n-pentyloxycarbonyl, 1,1-dimethyl-n-butoxycarbonyl, 1,2-dimethyl-n-butoxycarbonyl, 1,3-dimethyl-n-butoxycarbonyl, 2,2-dimethyl-n-butoxycarbonyl, 2,3-dimethyl-n-butoxycarbonyl, 3,3-dimethyl-n-butoxycarbonyl, 1-ethyl-n-butoxycarbonyl, 2-ethyl-n-butoxycarbonyl, 1,1,2-trimethyl-n-propoxycarbonyl, 1,2,2-trimethyl-n-propoxycarbonyl, 1-ethyl-1-methyl-n-propoxycarbonyl, 1-ethyl-2-methyl-n-propoxycarbonyl, c-hexyloxycarbonyl, 1-methyl-c-pentyloxycarbonyl, 2-methyl-c-pentyloxycarbonyl, 3-methyl-c-pentyloxycarbonyl, 1-ethyl-c-butoxycarbonyl, 2-ethyl-c-butoxycarbonyl, 3-ethyl-c-butoxycarbonyl, 1,2-dimethyl-c-butoxycarbonyl, 1,3-dimethyl-c-butoxycarbonyl, 2,2-dimethyl-c-butoxycarbonyl, 2,3-dimethyl-c-butoxycarbonyl, 2,4-dimethyl-c-butoxycarbonyl, 3,3-dimethyl-c-butoxycarbonyl, 1-n-propyl-c-propoxycarbonyl, 2-n-propyl-c-propoxycarbonyl, 1-i-propyl-c-propoxycarbonyl, 2-i-propyl-c-propoxycarbonyl, 1,2,2-trimethyl-c-propoxycarbonyl, 1,2,3-trimethyl-c-propoxycarbonyl, 2,2,3-trimethyl-c-propoxycarbonyl, 1-ethyl-2-methyl-c-propoxycarbonyl, 2-ethyl-1-methyl-c-propoxycarbonyl, 2-ethyl-2-methyl-c-propoxycarbonyl, 2-ethyl-3-methyl-c-propoxycarbonyl, 1-methyl-1-ethyl-n-pentyloxycarbonyl, 1-heptyloxycarbonyl, 2-heptyloxycarbonyl, 1-ethyl-1,2-dimethyl-n-propyloxycarbonyl, 1-ethyl-2,2-dimethyl-n-propyloxycarbonyl, 1-octyloxycarbonyl, 3-octyloxycarbonyl, 4-methyl-3-n-heptyloxycarbonyl, 6-methyl-2-n-heptyloxycarbonyl, 2-propyl-1-n-heptyloxycarbonyl, 2,4,4-trimethyl-1-n-pentyloxycarbonyl, 1-nonyloxycarbonyl, 2-nonyloxycarbonyl, 2,6-dimethyl-4-n-heptyloxycarbonyl, 3-ethyl-2,2-dimethyl-3-n-pentyloxycarbonyl, 3,5,5-trimethyl-1-n-hexyloxycarbonyl, 1-decyloxycarbonyl, 2-decyloxycarbonyl, 4-decyloxycarbonyl, 3,7-dimethyl-1-n-octyloxycarbonyl, 3,7-dimethyl-3-n-octyloxycarbonyl or the like may be mentioned.

A C₁₋₃ alkylcarbonyl group may linear, branched or a C₃ cycloalkylcarbonyl group, and as specific examples, methylcarbonyl, ethylcarbonyl, n-propylcarbonyl, i-propylcarbonyl, c-propylcarbonyl or the like may be mentioned.

A C₁₋₁₀ alkylcarbonyl group may linear, branched or a C₃₋₁₀ cycloalkylcarbonyl group, and as specific examples, in addition to those mentioned above, n-butylcarbonyl, i-butylcarbonyl, s-butylcarbonyl, t-butylcarbonyl, c-butylcarbonyl, 1-methyl-c-propylcarbonyl, 2-methyl-c-propylcarbonyl, n-pentylcarbonyl, 1-methyl-n-butylcarbonyl, 2-methyl-n-butylcarbonyl, 3-methyl-n-butylcarbonyl, 1,1-dimethyl-n-propylcarbonyl, 1,2-dimethyl-n-propylcarbonyl, 2,2-dimethyl-n-propylcarbonyl, 1-ethyl-n-propylcarbonyl, c-pentylcarbonyl, 1-methyl-c-butylcarbonyl, 2-methyl-c-butylcarbonyl, 3-methyl-c-butylcarbonyl, 1,2-dimethyl-c-propylcarbonyl, 2,3-dimethyl-c-propylcarbonyl, 1-ethyl-c-propylcarbonyl, 2-ethyl-c-propylcarbonyl, n-hexylcarbonyl, 1-methyl-n-pentylcarbonyl, 2-methyl-n-pentylcarbonyl, 3-methyl-n-pentylcarbonyl, 4-methyl-n-pentylcarbonyl, 1,1-dimethyl-n-butylcarbonyl, 1,2-dimethyl-n-butylcarbonyl, 1,3-dimethyl-n-butylcarbonyl, 2,2-dimethyl-n-butylcarbonyl, 2,3-dimethyl-n-butylcarbonyl, 3,3-dimethyl-n-butylcarbonyl, 1-ethyl-n-butylcarbonyl, 2-ethyl-n-butylcarbonyl, 1,1,2-trimethyl-n-propylcarbonyl, 1,2,2-trimethyl-n-propylcarbonyl, 1-ethyl-1-methyl-n-propylcarbonyl, 1-ethyl-2-methyl-n-propylcarbonyl, c-hexylcarbonyl, 1-methyl-c-pentylcarbonyl, 2-methyl-c-pentylcarbonyl, 3-methyl-c-pentylcarbonyl, 1-ethyl-c-butylcarbonyl, 2-ethyl-c-butylcarbonyl, 3-ethyl-c-butylcarbonyl, 1,2-dimethyl-c-butylcarbonyl, 1,3-dimethyl-c-butylcarbonyl, 2,2-dimethyl-c-butylcarbonyl, 2,3-dimethyl-c-butylcarbonyl, 2,4-dimethyl-c-butylcarbonyl, 3,3-dimethyl-c-butylcarbonyl, 1-n-propyl-c-propylcarbonyl, 2-n-propyl-c-propylcarbonyl, 1-i-propyl-c-propylcarbonyl, 2-i-propyl-c-propylcarbonyl, 1,2,2-trimethyl-c-propylcarbonyl, 1,2,3-trimethyl-c-propylcarbonyl, 2,2,3-trimethyl-c-propylcarbonyl, 1-ethyl-2-methyl-c-propylcarbonyl, 2-ethyl-1-methyl-c-propylcarbonyl, 2-ethyl-2-methyl-c-propylcarbonyl, 2-ethyl-3-methyl-c-propylcarbonyl, 1-methyl-1-ethyl-n-pentylcarbonyl, 1-heptylcarbonyl, 2-heptylcarbonyl, 1-ethyl-1,2-dimethyl-n-propylcarbonyl, 1-ethyl-2,2-dimethyl-n-propylcarbonyl, 1-octylcarbonyl, 3-octylcarbonyl, 4-methyl-3-n-heptylcarbonyl, 6-methyl-2-n-heptylcarbonyl, 2-propyl-1-n-heptylcarbonyl, 2,4,4-trimethyl-1-n-pentylcarbonyl, 1-nonylcarbonyl, 2-nonylcarbonyl, 2,6-dimethyl-4-n-heptylcarbonyl, 3-ethyl-2,2-dimethyl-3-n-pentylcarbonyl, 3,5,5-trimethyl-1-n-hexylcarbonyl, 1-decylcarbonyl, 2-decylcarbonyl, 4-decylcarbonyl, 3,7-dimethyl-1-n-octylcarbonyl, 3,7-dimethyl-3-n-octylcarbonyl or the like may be mentioned.

A C₁₋₁₀ alkylcarbonyloxy group may be linear, branched or a C₃₋₁₀ cycloalkylcarbonyloxy group, and as specific examples, in addition to those mentioned above, n-butylcarbonyloxy, i-butylcarbonyloxy, s-butylcarbonyloxy, t-butylcarbonyloxy, c-butylcarbonyloxy, 1-methyl-c-propylcarbonyloxy, 2-methyl-c-propylcarbonyloxy, n-pentylcarbonyloxy, 1-methyl-n-butylcarbonyloxy, 2-methyl-n-butylcarbonyloxy, 3-methyl-n-butylcarbonyloxy, 1,1-dimethyl-n-propylcarbonyloxy, 1,2-dimethyl-n-propylcarbonyloxy, 2,2-dimethyl-n-propylcarbonyloxy, 1-ethyl-n-propylcarbonyloxy, c-pentylcarbonyloxy, 1-methyl-c-butylcarbonyloxy, 2-methyl-c-butylcarbonyloxy, 3-methyl-c-butylcarbonyloxy, 1,2-dimethyl-c-propylcarbonyloxy, 2,3-dimethyl-c-propylcarbonyloxy, 1-ethyl-c-propylcarbonyloxy, 2-ethyl-c-propylcarbonyloxy, n-hexylcarbonyloxy, 1-methyl-n-pentylcarbonyloxy, 2-methyl-n-pentylcarbonyloxy, 3-methyl-n-pentylcarbonyloxy, 4-methyl-n-pentylcarbonyloxy, 1,1-dimethyl-n-butylcarbonyloxy, 1,2-dimethyl-n-butylcarbonyloxy, 1,3-dimethyl-n-butylcarbonyloxy, 2,2-dimethyl-n-butylcarbonyloxy, 2,3-dimethyl-n-butylcarbonyloxy, 3,3-dimethyl-n-butylcarbonyloxy, 1-ethyl-n-butylcarbonyloxy, 2-ethyl-n-butylcarbonyloxy, 1,1,2-trimethyl-n-propylcarbonyloxy, 1,2,2-trimethyl-n-propylcarbonyloxy, 1-ethyl-1-methyl-n-propylcarbonyloxy, 1-ethyl-2-methyl-n-propylcarbonyloxy, c-hexylcarbonyloxy, 1-methyl-c-pentylcarbonyloxy, 2-methyl-c-pentylcarbonyloxy, 3-methyl-c-pentylcarbonyloxy, 1-ethyl-c-butylcarbonyloxy, 2-ethyl-c-butylcarbonyloxy, 3-ethyl-c-butylcarbonyloxy, 1,2-dimethyl-c-butylcarbonyloxy, 1,3-dimethyl-c-butylcarbonyloxy, 2,2-dimethyl-c-butylcarbonylxoy, 2,3-dimethyl-c-butylcarbonyloxy, 2,4-dimethyl-c-butylcarbonyloxy, 3,3-dimethyl-c-butylcarbonyloxy, 1-n-propyl-c-propylcarbonyloxy, 2-n-propyl-c-propylcarbonyloxy, 1-i-propyl-c-propylcarbonyloxy, 2-i-propyl-c-propylcarbonyloxy, 1,2,2-trimethyl-c-propylcarbonyloxy, 1,2,3-trimethyl-c-propylcarbonyloxy, 2,2,3-trimethyl-c-propylcarbonyloxy, 1-ethyl-2-methyl-c-propylcarbonyloxy, 2-ethyl-1-methyl-c-propylcarbonyloxy, 2-ethyl-2-methyl-c-propylcarbonyloxy, 2-ethyl-3-methyl-c-propylcarbonyloxy, 1-methyl-1-ethyl-n-pentylcarbonyloxy, 1-heptylcarbonyloxy, 2-heptylcarbonyloxy, 1-ethyl-1,2-dimethyl-n-propylcarbonyloxy, 1-ethyl-2,2-dimethyl-n-propylcarbonyloxy, 1-octylcarbonyloxy, 3-octylcarbonyloxy, 4-methyl-3-n-heptylcarbonyloxy, 6-methyl-2-n-heptylcarbonyloxy, 2-propyl-1-n-heptylcarbonyloxy, 2,4,4-trimethyl-1-n-pentylcarbonyloxy, 1-nonylcarbonyloxy, 2-nonylcarbonyloxy, 2,6-dimethyl-4-n-heptylcarbonyloxy, 3-ethyl-2,2-dimethyl-3-n-pentylcarbonyloxy, 3,5,5-trimethyl-1-n-hexylcarbonyloxy, 1-decylcarbonyloxy, 2-decylcarbonyloxy, 4-decylcarbonyloxy, 3,7-dimethyl-1-n-octylcarbonyloxy, 3,7-dimethyl-3-n-octylcarbonyloxy or the like may be mentioned.

A C₁₋₁₀ alkylcarbonylamino group may be linear, branched or a C₃₋₁₀ cycloalkylcarbonylamino group, and as specific examples, methylcarbonylamino, ethylcarbonylamino, n-propylcarbonylamino, i-propylcarbonylamino, c-propylcarbonylamino, n-butylcarbonylamino, i-butylcarbonylamino, s-butylcarbonylamino, t-butylcarbonylamino, c-butylcarbonylamino, 1-methyl-c-propylcarbonylamino, 2-methyl-c-propylcarbonylamino, n-pentylcarbonylamino, 1-methyl-n-butylcarbonylamino, 2-methyl-n-butylcarbonylamino, 3-methyl-n-butylcarbonylamino, 1,1-dimethyl-n-propylcarbonylamino, 1,2-dimethyl-n-propylcarbonylamino, 2,2-dimethyl-n-propylcarbonylamino, 1-ethyl-n-propylcarbonylamino, c-pentylcarbonylamino, 1-methyl-c-butylcarbonylamino, 2-methyl-c-butylcarbonylamino, 3-methyl-c-butylcarbonylamino, 1,2-dimethyl-c-propylcarbonylamino, 2,3-dimethyl-c-propylcarbonylamino, 1-ethyl-c-propylcarbonylamino, 2-ethyl-c-propylcarbonylamino, n-hexylcarbonylamino, 1-methyl-n-pentylcarbonylamino, 2-methyl-n-pentylcarbonylamino, 3-methyl-n-pentylcarbonylamino, 4-methyl-n-pentylcarbonylamino, 1,1-dimethyl-n-butylcarbonylamino, 1,2-dimethyl-n-butylcarbonylamino, 1,3-dimethyl-n-butylcarbonylamino, 2,2-dimethyl-n-butylcarbonylamino, 2,3-dimethyl-n-butylcarbonylamino, 3,3-dimethyl-n-butylcarbonylamino, 1-ethyl-n-butylcarbonylamino, 2-ethyl-n-butylcarbonylamino, 1,1,2-trimethyl-n-propylcarbonylamino, 1,2,2-trimethyl-n-propylcarbonylamino, 1-ethyl-1-methyl-n-propylcarbonylamino, 1-ethyl-2-methyl-n-propylcarbonylamino, c-hexylcarbonylamino, 1-methyl-c-pentylcarbonylamino, 2-methyl-c-pentylcarbonylamino, 3-methyl-c-pentylcarbonylamino, 1-ethyl-c-butylcarbonylamino, 2-ethyl-c-butylcarbonylamino, 3-ethyl-c-butylcarbonylamino, 1,2-dimethyl-c-butylcarbonylamino, 1,3-dimethyl-c-butylcarbonylamino, 2,2-dimethyl-c-butylcarbonylamino, 2,3-dimethyl-c-butylcarbonylamino, 2,4-dimethyl-c-butylcarbonylamino, 3,3-dimethyl-c-butylcarbonylamino, 1-n-propyl-c-propylcarbonylamino, 2-n-propyl-c-propylcarbonylamino, 1-i-propyl-c-propylcarbonylamino, 2-i-propyl-c-propylcarbonylamino, 1,2,2-trimethyl-c-propyl-carbonylamino, 1,2,3-trimethyl-c-propylcarbonylamino, 2,2,3-trimethyl-c-propylcarbonylamino, 1-ethyl-2-methyl-c-propylcarbonylamino, 2-ethyl-1-methyl-c-propylcarbonylamino, 2-ethyl-2-methyl-c-propylcarbonylamino, 2-ethyl-3-methyl-c-propylcarbonylamino, 1-methyl-1-ethyl-n-pentylcarbonylamino, 1-heptylcarbonylamino, 2-heptylcarbonylamino, 1-ethyl-1,2-dimethyl-n-propylcarbonylamino, 1-ethyl-2,2-dimethyl-n-propylcarbonylamino, 1-octylcarbonylamino, 3-octylcarbonylamino, 4-methyl-3-n-heptylcarbonylamino, 6-methyl-2-n-heptylcarbonylamino, 2-propyl-1-n-heptylcarbonylamino, 2,4,4-trimethyl-1-n-pentylcarbonylamino, 1-nonylcarbonylamino, 2-nonylcarbonylamino, 2,6-dimethyl-4-n-heptylcarbonylamino, 3-ethyl-2,2-dimethyl-3-n-pentylcarbonylamino, 3,5,5-trimethyl-1-n-hexylcarbonylamino, 1-decylcarbonylamino, 2-decylcarbonylamino, 4-decylcarbonylamino, 3,7-dimethyl-1-n-octylcarbonylannino, 3,7-dimethyl-3-n-octylcarbonylamino or the like may be mentioned.

A C₁₋₁₀ monoalkylamino group may be linear, branched or a C₃₋₁₀ cycloalkylamino group, and specific examples, methylamino, ethylamino, n-propylamino, i-propylamino, c-propylamino, n-butylamino, i-butylamino, s-butylamino, t-butylamino, c-butylamino, 1-methyl-c-propylamino, 2-methyl-c-propylamino, n-pentylamino, 1-methyl-n-butylamino, 2-methyl-n-butylamino, 3-methyl-n-butylamino, 1,1-dimethyl-n-propylamino, 1,2-dimethyl-n-propylamino, 2,2-dimethyl-n-propylamino, 1-ethyl-n-propylamino, c-pentylamino, 1-methyl-c-butylamino, 2-methyl-c-butylamino, 3-methyl-c-butylamino, 1,2-dimethyl-c-propylamino, 2,3-dimethyl-c-propylamino, 1-ethyl-c-propylamino, 2-ethyl-c-propylamino, n-hexylamino, 1-methyl-n-pentylamino, 2-methyl-n-pentylamino, 3-methyl-n-pentylamino, 4-methyl-n-pentylamino, 1,1-dimethyl-n-butylamino, 1,2-dimethyl-n-butylamino, 1,3-dimethyl-n-butylamino, 2,2-dimethyl-n-butylamino, 2,3-dimethyl-n-butylamino, 3,3-dimethyl-n-butylamino, 1-ethyl-n-butylamino, 2-ethyl-n-butylamino, 1,1,2-trimethyl-n-propylamino, 1,2,2-trimethyl-n-propylamino, 1-ethyl-1-methyl-n-propylamino, 1-ethyl-2-methyl-n-propylamino, c-hexylamino, 1-methyl-c-pentylamino, 2-methyl-c-pentylamino, 3-methyl-c-pentylamino, 1-ethyl-c-butylamino, 2-ethyl-c-butylamino, 3-ethyl-c-butylamino, 1,2-dimethyl-c-butylamino, 1,3-dimethyl-c-butylamino, 2,2-dimethyl-c-butylamino, 2,3-dimethyl-c-butylamino, 2,4-dimethyl-c-butylamino, 3,3-dimethyl-c-butylamino, 1-n-propyl-c-propylamino, 2-n-propyl-c-propylamino, 1-i-propyl-c-propylamino, 2-i-propyl-c-propylamino, 1,2,2-trimethyl-c-propylamino, 1,2,3-trimethyl-c-propylamino, 2,2,3-trimethyl-c-propylamino, 1-ethyl-2-methyl-c-propylamino, 2-ethyl-1-methyl-c-propylamino, 2-ethyl-2-methyl-c-propylamino, 2-ethyl-3-methyl-c-propylamino, 1-methyl-1-ethyl-n-pentylamino, 1-heptylamino, 2-heptylamino, 1-ethyl-1,2-dimethyl-n-propylamino, 1-ethyl-2,2-dimethyl-n-propylamino, 1-octylamino, 3-octylamino, 4-methyl-3-n-heptylamino, 6-methyl-2-n-heptylamino, 2-propyl-1-n-heptylamino, 2,4,4-trimethyl-1-n-pentylamino, 1-nonylamino, 2-nonylamino, 2,6-dimethyl-4-n-heptylamino, 3-ethyl-2,2-dimethyl-3-n-pentylamino, 3,5,5-trimethyl-1-n-hexylamino, 1-decylamino, 2-decylamino, 4-decylamino, 3,7-dimethyl-1-n-octylamino, 3,7-dimethyl-3-n-octylamino or the like may be mentioned.

A di-C₁₋₁₀ alkylamino group may be symmetric or asymmetric. A symmetric di-C₁₋₁₀ alkylamino group may be linear, branched or a C₃₋₁₀ cycloalkylamino group, and as specific examples, dimethylamino, diethylamino, di-n-propylamino, di-1-propylamino, di-c-propylamino, di-n-butylamino, di-1-butylamino, di-s-butylamino, di-t-butylamino, di-c-butylamino, di-(1-methyl-c-propyl)amino, di-(2-methyl-c-propyl)amino, di-n-pentylamino, di-(1-methyl-n-butyl)amino, di-(2-methyl-n-butyl)amino, di-(3-methyl-n-butyl)amino, di-(1,1-dimethyl-n-propyl)amino, di-(1,2-dimethyl-n-propyl)amino, di-(2,2-dimethyl-n-propyl)amino, di-(1-ethyl-n-propyl)amino, di-c-pentylamino, di-(1-methyl-c-butyl)amino, di-(2-methyl-c-butyl)amino, di-(3-methyl-c-butyl)amino, di-(1,2-dimethyl-c-propyl)amino, di-(2,3-dimethyl-c-propyl)amino, di-(1-ethyl-c-propyl)amino, di-(2-ethyl-c-propyl)amino, di-n-hexylamino, di-(1-methyl-n-pentyl)amino, di-(2-methyl-n-pentyl)amino, di-(3-methyl-n-pentyl)amino, di-(4-methyl-n-pentyl)amino, di-(1,1-dimethyl-n-butyl)amino, di-(1,2-dimethyl-n-butyl)amino, di-(1,3-dimethyl-n-butyl)amino, di-(2,2-dimethyl-n-butyl)amino, di-(2,3-dimethyl-n-butyl)amino, di-(3,3-dimethyl-n-butyl)amino, di-(1-ethyl-n-butyl)amino, di-(2-ethyl-n-butyl)amino, di-(1,1,2-trimethyl-n-propyl)amino, di-(1,2,2-trimethyl-n-propyl)amino, di-(1-ethyl-1-methyl-n-propyl)amino, di-(1-ethyl-2-methyl-n-propyl)amino, di-c-hexylamino, di-(1-methyl-c-pentyl)amino, di-(2-methyl-c-pentyl)amino, di-(3-methyl-c-pentyl)amino, di-(1-ethyl-c-butyl)amino, di-(2-ethyl-c-butyl)amino, di-(3-ethyl-c-butyl)amino, di-(1,2-dimethyl-c-butyl)amino, di-(1,3-dimethyl-c-butyl)amino, di-(2,2-dimethyl-c-butyl)amino, di-(2,3-dimethyl-c-butyl)amino, di-(2,4-dimethyl-c-butyl)amino, di-(3,3-dimethyl-c-butyl)amino, di-(1-n-propyl-c-propyl)amino, di-(2-n-propyl-c-propyl)amino, di-(1-i-propyl-c-propyl)amino, di-(2-i-propyl-c-propyl)amino, di-(1,2,2-trimethyl-c-propyl)amino, di-(1,2,3-trimethyl-c-propyl)amino, di-(2,2,3-trimethyl-c-propyl)amino, di-(1-ethyl-2-methyl-c-propyl)amino, di-(2-ethyl-1-methyl-c-propyl)amino, di-(2-ethyl-2-methyl-c-propyl)amino, di-(2-ethyl-3-methyl-c-propyl)amino, di-(1-methyl-1-ethyl-n-pentyl)amino, di-(1-heptyl)amino, di-(2-heptyl)amino, di-(1-ethyl-1,2-dimethyl-n-propyl)amino, di-(1-ethyl-2,2-dimethyl-n-propyl)amino, di-(1-octyl)amino, di-(3-octyl)amino, di-(4-methyl-3-n-heptyl)amino, di-(6-methyl-2-n-heptyl)amino, di-(2-propyl-1-n-heptyl)amino, di-(2,4,4-trimethyl-1-n-pentyl)amino, di-(1-nonyl)amino, di-(2-nonyl)amino, di-(2,6-dimethyl-4-n-heptyl)amino, di-(3-ethyl-2,2-dimethyl-3-n-pentyl)amino, di-(3,5,5-trimethyl-1-n-hexylamino, di-(1-decyl)amino, di-(2-decyl)amino, di-(4-decyl)amino, di-(3,7-dimethyl-1-n-octyl)amino, di-(3,7-dimethyl-3-n-octyl)amino or the like may be mentioned.

An asymmetric di-C₁₋₁₀ alkylamino group may be linear, branched or a C₃₋₁₀ cycloalkylamino group, and as specific examples, (methyl, ethyl)amino, (methyl, n-propyl)amino, (methyl, i-propyl)amino, (methyl, c-propyl)amino, (methyl, n-butyl)amino, (methyl, i-butyl)amino, (methyl, s-butyl)amino, (methyl, t-butyl)amino, (methyl, n-pentyl)amino, (methyl, c-pentyl)amino, (methyl, n-hexyl)amino, (methyl, c-hexyl)amino, (ethyl, n-propyl)amino, (ethyl, i-propyl)amino, (ethyl, c-propyl)amino, (ethyl, n-butyl)amino, (ethyl, i-butyl)amino, (ethyl, s-butyl)amino, (ethyl, t-butyl)amino, (ethyl, n-pentyl)amino, (ethyl, c-pentyl)amino, (ethyl, n-hexyl)amino, (ethyl, c-hexyl)amino, (n-propyl, i-propyl)amino, (n-propyl, c-propyl)amino, (n-propyl, n-butyl)amino, (n-propyl, i-butyl)amino, (n-propyl, s-butyl)amino, (n-propyl, t-butyl)amino, (n-propyl, n-pentyl)amino, (n-propyl, c-pentyl)amino, (n-propyl, n-hexyl)amino, (n-propyl, c-hexyl)amino, (i-propyl, c-propyl)amino, (i-propyl, n-butyl)amino, (i-propyl, i-butyl)amino, (i-propyl, s-butyl)amino, (i-propyl, t-butyl)amino, (i-propyl, n-pentyl)amino, (i-propyl, c-pentyl)amino, (i-propyl, n-hexyl)amino, (i-propyl, c-hexyl)amino, (c-propyl, n-butyl)amino, (c-propyl, i-butyl)amino, (c-propyl, s-butyl)amino, (c-propyl, t-butyl)amino, (c-propyl, n-pentyl)amino, (c-propyl, c-pentyl)amino, (c-propyl, n-hexyl)amino, (c-propyl, c-hexyl)amino, (n-butyl, i-butyl)amino, (n-butyl, s-butyl)amino, (n-butyl, t-butyl)amino, (n-butyl, n-pentyl)amino, (n-butyl, c-pentyl)amino, (n-butyl, n-hexyl)amino, (n-butyl, c-hexyl)amino, (i-butyl, s-butyl)amino, (i-butyl, t-butyl)amino, (i-butyl, n-pentyl)amino, (i-butyl, c-pentyl)amino, (i-butyl, n-hexyl)amino, (i-butyl, c-hexyl)amino, (s-butyl, t-butyl)amino, (s-butyl, n-pentyl)amino, (s-butyl, c-pentyl)amino, (s-butyl, n-hexyl)amino, (s-butyl, c-hexyl)amino, (t-butyl, n-pentyl)amino, (t-butyl, c-pentyl)amino, (t-butyl, n-hexyl)amino, (t-butyl, c-hexyl)amino, (n-pentyl, c-pentyl)amino, (n-pentyl, n-hexyl)amino, (n-pentyl, c-hexyl)amino, (c-pentyl, n-hexyl)amino, (c-pentyl, c-hexyl)amino, (n-hexyl, c-hexyl)amino, (methyl, n-heptyl)amino, (methyl, n-octyl)amino, (methyl, n-nonanyl)amino, (methyl, n-decyl)amino, (ethyl, n-heptyl)amino, (ethyl, n-octyl)amino, (ethyl, n-nonanyl)amino, (ethyl, n-decyl)amino or the like may be mentioned.

A C₁₋₁₀ alkylaminocarbonyl group may be linear, branched or a C₁₋₁₀ cycloalkylaminocarbonyl group and may be a di-C₁₋₁₀ alkylaminocarbonyl group, and as specific examples, methylaminocarbonyl, ethylaminocarbonyl, n-propylaminocarbonyl, propylaminocarbonyl, c-propylaminocarbonyl, n-butylaminocarbonyl, butylaminocarbonyl, s-butylaminocarbonyl, t-butylaminocarbonyl, c-butylaminocarbonyl, 1-methyl-c-propylaminocarbonyl, 2-methyl-c-propylaminocarbonyl, n-pentylaminocarbonyl, 1-methyl-n-butylaminocarbonyl, 2-methyl-n-butylaminocarbonyl, 3-methyl-n-butylaminocarbonyl, 1,1-dimethyl-n-propylaminocarbonyl, 1,2-dimethyl-n-propylaminocarbonyl, 2,2-dimethyl-n-propylaminocarbonyl, 1-ethyl-n-propylaminocarbonyl, c-pentylaminocarbonyl, 1-methyl-c-butylaminocarbonyl, 2-methyl-c-butylaminocarbonyl, 3-methyl-c-butylaminocarbonyl, 1,2-dimethyl-c-propylaminocarbonyl, 2,3-dimethyl-c-propylaminocarbonyl, 1-ethyl-c-propylaminocarbonyl, 2-ethyl-c-propylaminocarbonyl, n-hexylaminocarbonyl, 1-methyl-n-pentylaminocarbonyl, 2-methyl-n-pentylaminocarbonyl, 3-methyl-n-pentylaminocarbonyl, 4-methyl-n-pentylaminocarbonyl, 1,1-dimethyl-n-butylaminocarbonyl, 1,2-dimethyl-n-butylaminocarbonyl, 1,3-dimethyl-n-butylaminocarbonyl, 2,2-dimethyl-n-butylaminocarbonyl, 2,3-dimethyl-n-butylaminocarbonyl, 3,3-dimethyl-n-butylaminocarbonyl, 1-ethyl-n-butylaminocarbonyl, 2-ethyl-n-butylaminocarbonyl, 1,1,2-trimethyl-n-propylaminocarbonyl, 1,2,2-trimethyl-n-propylaminocarbonyl, 1-ethyl-1-methyl-n-propylaminocarbonyl, 1-ethyl-2-methyl-n-propylaminocarbonyl, c-hexylaminocarbonyl, 1-methyl-c-pentylaminocarbonyl, 2-methyl-c-pentylaminocarbonyl, 3-methyl-c-pentylaminocarbonyl, 1-ethyl-c-butylaminocarbonyl, 2-ethyl-c-butylaminocarbonyl, 3-ethyl-c-butylaminocarbonyl, 1,2-dimethyl-c-butylaminocarbonyl, 1,3-dimethyl-c-butylaminocarbonyl, 2,2-dimethyl-c-butylaminocarbonyl, 2,3-dimethyl-c-butylaminocarbonyl, 2,4-dimethyl-c-butylaminocarbonyl, 3,3-dimethyl-c-butylaminocarbonyl, 1-n-propyl-c-propylaminocarbonyl, 2-n-propyl-c-propylaminocarbonyl, 1-i-propyl-c-propylaminocarbonyl, 2-i-propyl-c-propylaminocarbonyl, 1,2,2-trimethyl-c-propylaminocarbonyl, 1,2,3-trimethyl-c-propylaminocarbonyl, 2,2,3-trimethyl-c-propylaminocarbonyl, 1-ethyl-2-methyl-c-propylaminocarbonyl, 2-ethyl-1-methyl-c-propylaminocarbonyl, 2-ethyl-2-methyl-c-propylaminocarbonyl, 2-ethyl-3-methyl-c-propylaminocarbonyl, 1-methyl-1-ethyl-n-pentylaminocarbonyl, 1-heptylaminocarbonyl, 2-heptylaminocarbonyl, 1-ethyl-1,2-dimethyl-n-propylaminocarbonyl, 1-ethyl-2,2-dimethyl-n-propylaminocarbonyl, 1-octylaminocarbonyl, 3-octylaminocarbonyl, 4-methyl-3-n-heptylaminocarbonyl, 6-methyl-2-n-heptylaminocarbonyl, 2-propyl-1-n-heptylaminocarbonyl, 2,4,4-trimethyl-1-n-pentylaminocarbonyl, 1-nonylaminocarbonyl, 2-nonylaminocarbonyl, 2,6-dimethyl-4-n-heptylaminocarbonyl, 3-ethyl-2,2-dimethyl-3-n-pentylaminocarbonyl, 3,5,5-trimethyl-1-n-hexylaminocarbonyl, 1-decylaminocarbonyl, 2-decylaminocarbonyl, 4-decylaminocarbonyl, 3,7-dimethyl-1-n-octylaminocarbonyl, 3,7-dimethyl-3-n-octylaminocarbonyl or the like may be mentioned.

A di-C₁₋₁₀ alkylaminocarbonyl group may be symmetric or asymmetric. A symmetric di-C₁₋₁₀ alkylaminocarbonyl group may be linear, branched or a C₃₋₁₀ cycloalkylaminocarbonyl group, and as specific examples, dimethylaminocarbonyl, diethylaminocarbonyl, di-n-propylaminocarbonyl, di-1-propylaminocarbonyl, di-c-propylaminocarbonyl, di-n-butylaminocarbonyl, di-1-butylaminocarbonyl, di-s-butylaminocarbonyl, di-t-butylaminocarbonyl, di-c-butylaminocarbonyl, di-(1-methyl-c-propyl)aminocarbonyl, di-(2-methyl-c-propyl)aminocarbonyl, di-n-pentylaminocarbonyl, di-(1-methyl-n-butyl)aminocarbonyl, di-(2-methyl-n-butyl)aminocarbonyl, di-(3-methyl-n-butyl)aminocarbonyl, di-(1,1-dimethyl-n-propyl)aminocarbonyl, di-(1,2-dimethyl-n-propyl)aminocarbonyl, di-(2,2-dimethyl-n-propyl)aminocarbonyl, di-(1-ethyl-n-propyl)aminocarbonyl, di-c-pentylaminocarbonyl, di-(1-methyl-c-butyl)aminocarbonyl, di-(2-methyl-c-butyl)aminocarbonyl, di-(3-methyl-c-butyl)aminocarbonyl, di-(1,2-dimethyl-c-propyl)aminocarbonyl, di-(2,3-dimethyl-c-propyl)aminocarbonyl, di-(1-ethyl-c-propyl)aminocarbonyl, di-(2-ethyl-c-propyl)aminocarbonyl, di-n-hexylaminocarbonyl, di-(1-methyl-n-pentyl)aminocarbonyl, di-(2-methyl-n-pentyl)aminocarbonyl, di-(3-methyl-n-pentyl)aminocarbonyl, di-(4-methyl-n-pentyl)aminocarbonyl, di-(1,1-dimethyl-n-butyl)aminocarbonyl, di-(1,2-dimethyl-n-butyl)aminocarbonyl, di-(1,3-dimethyl-n-butyl)aminocarbonyl, di-(2,2-dimethyl-n-butyl)aminocarbonyl, di-(2,3-dimethyl-n-butyl)aminocarbonyl, di-(3,3-dimethyl-n-butyl)aminocarbonyl, di-(1-ethyl-n-butyl)aminocarbonyl, di-(2-ethyl-n-butyl)aminocarbonyl, di-(1,1,2-trimethyl-n-propyl)aminocarbonyl, di-(1,2,2-trimethyl-n-propyl)aminocarbonyl, di-(1-ethyl-1-methyl-n-propyl)aminocarbonyl, di-(1-ethyl-2-methyl-n-propyl)aminocarbonyl, di-c-hexylaminocarbonyl, di-(1-methyl-c-pentyl)aminocarbonyl, di-(2-methyl-c-pentyl)aminocarbonyl, di-(3-methyl-c-pentyl)aminocarbonyl, di-(1-ethyl-c-butyl)aminocarbonyl, di-(2-ethyl-c-butyl)aminocarbonyl, di-(3-ethyl-c-butyl)aminocarbonyl, di-(1,2-dimethyl-c-butyl)aminocarbonyl, di-(1,3-dimethyl-c-butyl)aminocarbonyl, di-(2,2-dimethyl-c-butyl)aminocarbonyl, di-(2,3-dimethyl-c-butyl)aminocarbonyl, di-(2,4-dimethyl-c-butyl)aminocarbonyl, di-(3,3-dimethyl-c-butyl)aminocarbonyl, di-(1-n-propyl-c-propyl)aminocarbonyl, di-(2-n-propyl-c-propyl)aminocarbonyl, di-(1-i-propyl-c-propyl)aminocarbonyl, di-(2-i-propyl-c-propyl)aminocarbonyl, di-(1,2,2-trimethyl-c-propyl)aminocarbonyl, di-(1,2,3-trimethyl-c-propyl)aminocarbonyl, di-(2,2,3-trimethyl-c-propyl)aminocarbonyl, di-(1-ethyl-2-methyl-c-propyl)aminocarbonyl, di-(2-ethyl-1-methyl-c-propyl)aminocarbonyl, di-(2-ethyl-2-methyl-c-propyl)aminocarbonyl, di-(2-ethyl-3-methyl-c-propyl)aminocarbonyl, di-(1-methyl-1-ethyl-n-pentyl)aminocarbonyl, di-(1-heptyl)aminocarbonyl, di-(2-heptyl)aminocarbonyl, di-(1-ethyl-1,2-dimethyl-n-propyl)aminocarbonyl, di-(1-ethyl-2,2-dimethyl-n-propyl)aminocarbonyl, di-(1-octyl)aminocarbonyl, di-(3-octyl)aminocarbonyl, di-(4-methyl-3-n-heptyl)aminocarbonyl, di-(6-methyl-2-n-heptyl)aminocarbonyl, di-(2-propyl-1-n-heptyl)aminocarbonyl, di-(2,4,4-trimethyl-1-n-pentyl)aminocarbonyl, di-(1-nonyl)aminocarbonyl, di-(2-nonyl)aminocarbonyl, di-(2,6-dimethyl-4-n-heptyl)aminocarbonyl, di-(3-ethyl-2,2-dimethyl-3-n-pentyl)aminocarbonyl, di-(3,5,5-trimethyl-1-n-hexyl)aminocarbonyl, di-(1-decyl)aminocarbonyl, di-(2-decyl)aminocarbonyl, di-(4-decyl)aminocarbonyl, di-(3,7-dimethyl-1-n-octyl)aminocarbonyl, di-(3,7-dimethyl-3-n-octyl)aminocarbonyl or the like may be mentioned.

An asymmetric C₁₋₁₀ dialkylaminocarbonyl group may be linear, branched or a C₃₋₁₀ cycloalkylaminocarbonyl group, and as specific examples, (methyl, ethyl)aminocarbonyl, (methyl, n-propyl)aminocarbonyl, (methyl, i-propyl)aminocarbonyl, (methyl, c-propyl)aminocarbonyl, (methyl, n-butyl)aminocarbonyl, (methyl, i-butyl)aminocarbonyl, (methyl, s-butyl)aminocarbonyl, (methyl, t-butyl)aminocarbonyl, (methyl, n-pentyl)aminocarbonyl, (methyl, c-pentyl)aminocarbonyl, (methyl, n-hexyl)aminocarbonyl, (methyl, c-hexyl)aminocarbonyl, (ethyl, n-propyl)aminocarbonyl, (ethyl, i-propyl)aminocarbonyl, (ethyl, c-propyl)aminocarbonyl, (ethyl, n-butyl)aminocarbonyl, (ethyl, i-butyl)aminocarbonyl, (ethyl, s-butyl)aminocarbonyl, (ethyl, t-butyl)aminocarbonyl, (ethyl, n-pentyl)aminocarbonyl, (ethyl, c-pentyl)aminocarbonyl, (ethyl, n-hexyl)aminocarbonyl, (ethyl, c-hexyl)aminocarbonyl, (n-propyl, i-propyl)aminocarbonyl, (n-propyl, c-propyl)aminocarbonyl, (n-propyl, n-butyl)aminocarbonyl, (n-propyl, i-butyl)aminocarbonyl, (n-propyl, s-butyl)aminocarbonyl, (n-propyl, t-butyl)aminocarbonyl, (n-propyl, n-pentyl)aminocarbonyl, (n-propyl, c-pentyl)aminocarbonyl, (n-propyl, n-hexyl)aminocarbonyl, (n-propyl, c-hexyl)aminocarbonyl, (i-propyl, c-propyl)aminocarbonyl, (i-propyl, n-butyl)aminocarbonyl, (i-propyl, i-butyl)aminocarbonyl, (i-propyl, s-butyl)aminocarbonyl, (i-propyl, t-butyl)aminocarbonyl, (i-propyl, n-pentyl)aminocarbonyl, (i-propyl, c-pentyl)aminocarbonyl, (i-propyl, n-hexyl)aminocarbonyl, (i-propyl, c-hexyl)aminocarbonyl, (c-propyl, n-butyl)aminocarbonyl, (c-propyl, i-butyl)aminocarbonyl, (c-propyl, s-butyl)aminocarbonyl, (c-propyl, t-butyl)aminocarbonyl, (c-propyl, n-pentyl)aminocarbonyl, (c-propyl, c-pentyl)aminocarbonyl, (c-propyl, n-hexyl)aminocarbonyl, (c-propyl, c-hexyl)aminocarbonyl, (n-butyl, i-butyl)aminocarbonyl, (n-butyl, s-butyl)aminocarbonyl, (n-butyl, t-butyl)aminocarbonyl, (n-butyl, n-pentyl)aminocarbonyl, (n-butyl, c-pentyl)aminocarbonyl, (n-butyl, n-hexyl)aminocarbonyl, (n-butyl, c-hexyl)aminocarbonyl, (i-butyl, s-butyl)aminocarbonyl, (i-butyl, t-butyl)aminocarbonyl, (i-butyl, n-pentyl)aminocarbonyl, (i-butyl, c-pentyl)aminocarbonyl, (i-butyl, n-hexyl)aminocarbonyl, (i-butyl, c-hexyl)aminocarbonyl, (s-butyl, t-butyl)aminocarbonyl, (s-butyl, n-pentyl)aminocarbonyl, (s-butyl, c-pentyl)aminocarbonyl, (s-butyl, n-hexyl)aminocarbonyl, (s-butyl, c-hexyl)aminocarbonyl, (t-butyl, n-pentyl)aminocarbonyl, (t-butyl, c-pentyl)aminocarbonyl, (t-butyl, n-hexyl)aminocarbonyl, (t-butyl, c-hexyl)aminocarbonyl, (n-pentyl, c-pentyl)aminocarbonyl, (n-pentyl, n-hexyl)aminocarbonyl, (n-pentyl, c-hexyl)aminocarbonyl, (c-pentyl, n-hexyl)aminocarbonyl, (c-pentyl, c-hexyl)aminocarbonyl, (n-hexyl, c-hexyl)aminocarbonyl, (methyl, n-heptyl)aminocarbonyl, (methyl, n-octyl)aminocarbonyl, (methyl, n-nonanyl)aminocarbonyl, (methyl, n-decyl)aminocarbonyl, (ethyl, n-heptyl)aminocarbonyl, (ethyl, n-octyl)aminocarbonyl, (ethyl, n-nonanyl)aminocarbonyl, (ethyl, n-decyl)aminocarbonyl or the like may be mentioned.

A C₁₋₁₀ alkylaminosulfonyl group may be linear, branched, a C₃₋₁₀ cycloalkylsulfonylamino group or a di-C₁₋₁₀ alkylaminosulfonyl group, and as specific examples, methylaminosulfonyl, ethylaminosulfonyl, n-propylaminosulfonyl, i-propylaminosulfonyl, c-propylaminosulfonyl, n-butylaminosulfonyl, i-butylaminosulfonyl, s-butylaminosulfonyl, t-butylaminosulfonyl, c-butylaminosulfonyl, 1-methyl-c-propylaminosulfonyl, 2-methyl-c-propylaminosulfonyl, n-pentylaminosulfonyl, 1-methyl-n-butylaminosulfonyl, 2-methyl-n-butylaminosulfonyl, 3-methyl-n-butylaminosulfonyl, 1,1-dimethyl-n-propylaminosulfonyl, 1,2-dimethyl-n-propylaminosulfonyl, 2,2-dimethyl-n-propylaminosulfonyl, 1-ethyl-n-propylaminosulfonyl, c-pentylaminosulfonyl, 1-methyl-c-butylaminosulfonyl, 2-methyl-c-butylaminosulfonyl, 3-methyl-c-butylaminosulfonyl, 1,2-dimethyl-c-propylaminosulfonyl, 2,3-dimethyl-c-propylaminosulfonyl, 1-ethyl-c-propylaminosulfonyl, 2-ethyl-c-propylaminosulfonyl, n-hexylaminosulfonyl, 1-methyl-n-pentylaminosulfonyl, 2-methyl-n-pentylaminosulfonyl, 3-methyl-n-pentylaminosulfonyl, 4-methyl-n-pentylaminosulfonyl, 1,1-dimethyl-n-butylaminosulfonyl, 1,2-dimethyl-n-butylaminosulfonyl, 1,3-dimethyl-n-butylaminosulfonyl, 2,2-dimethyl-n-butylaminosulfonyl, 2,3-dimethyl-n-butylaminosulfonyl, 3,3-dimethyl-n-butylaminosulfonyl, 1-ethyl-n-butylaminosulfonyl, 2-ethyl-n-butylaminosulfonyl, 1,1,2-trimethyl-n-propylaminosulfonyl, 1,2,2-trimethyl-n-propylaminosulfonyl, 1-ethyl-1-methyl-n-propylaminosulfonyl, 1-ethyl-2-methyl-n-propylaminosulfonyl, c-hexylaminosulfonyl, 1-methyl-c-pentylaminosulfonyl, 2-methyl-c-pentylaminosulfonyl, 3-methyl-c-pentylaminosulfonyl, 1-ethyl-c-butylaminosulfonyl, 2-ethyl-c-butylaminosulfonyl, 3-ethyl-c-butylaminosulfonyl, 1,2-dimethyl-c-butylaminosulfonyl, 1,3-dimethyl-c-butylaminosulfonyl, 2,2-dimethyl-c-butylaminosulfonyl, 2,3-dimethyl-c-butylaminosulfonyl, 2,4-dimethyl-c-butylaminosulfonyl, 3,3-dimethyl-c-butylaminosulfonyl, 1-n-propyl-c-propylaminosulfonyl, 2-n-propyl-c-propylaminosulfonyl, 1-i-propyl-c-propylaminosulfonyl, 2-i-propyl-c-propylaminosulfonyl, 1,2,2-trimethyl-c-propylaminosulfonyl, 1,2,3-trimethyl-c-propylaminosulfonyl, 2,2,3-trimethyl-c-propylaminosulfonyl, 1-ethyl-2-methyl-c-propylaminosulfonyl, 2-ethyl-1-methyl-c-propylaminosulfonyl, 2-ethyl-2-methyl-c-propylaminosulfonyl, 1-methyl-1-ethyl-n-pentylaminosulfonyl, 1-heptylaminosulfonyl, 2-heptylaminosulfonyl, 1-ethyl-1,2-dimethyl-n-propylaminosulfonyl, 1-ethyl-2,2-dimethyl-n-propylaminosulfonyl, 1-octylaminosulfonyl, 3-octylaminosulfonyl, 4-methyl-3-n-heptylaminosulfonyl, 6-methyl-2-n-heptylaminosulfonyl, 2-propyl-1-n-heptylaminosulfonyl, 2,4,4-trimethyl-1-n-pentylaminosulfonyl, 1-nonylaminosulfonyl, 2-nonylaminosulfonyl, 2,6-dimethyl-4-n-heptylaminosulfonyl, 3-ethyl-2,2-dimethyl-3-n-pentylaminosulfonyl, 3,5,5-trimethl-1-n-hexylaminosulfonyl, 1-decylaminosulfonyl, 2-decylaminosulfonyl, 4-decylaminosulfonyl, 3,7-dimethyl-1-n-octylaminosulfonyl, 3,7-dimethyl-3-n-octylaminosulfonyl, c-heptylaminosulfonyl, c-octylaminosulfonyl, 1-methyl-c-hexylaminosulfonyl, 2-methyl-c-hexylaminosulfonyl, 3-methyl-c-hexylaminosulfonyl, 1,2-dimethyl-c-hexylaminosulfonyl, 1-ethyl-c-hexylaminosulfonyl, 1-methyl-c-pentylaminosulfonyl, 2-methyl-c-pentylaminosulfonyl, 3-methyl-c-pentylaminosulfonyl or the like may be mentioned.

A di-C₁₋₁₀ alkylaminosulfonyl group may be symmetric or asymmetric. A symmetric di-C₁₋₁₀ dialkylaminosulfonyl group may be linear, branched or a C₃₋₁₀ cycloalkylaminosulfonyl group, and as specific examples, dimethylaminosulfonyl, diethylaminosulfonyl, di-n-propylaminosulfonyl, di-1-propylaminosulfonyl, di-c-propylaminosulfonyl, di-n-butylaminosulfonyl, di-1-butylaminosulfonyl, di-s-butylaminosulfonyl, di-t-butylaminosulfonyl, di-c-butylaminosulfonyl, di-(1-methyl-c-propyl)aminosulfonyl, di-(2-methyl-c-propyl)aminosulfonyl, di-n-pentylaminosulfonyl, di-(1-methyl-n-butyl)aminosulfonyl, di-(2-methyl-n-butyl)aminosulfonyl, di-(3-methyl-n-butyl)aminosulfonyl, di-(1,1-dimethyl-n-propyl)aminosulfonyl, di-(1,2-dimethyl-n-propyl)aminosulfonyl, di-(2,2-dimethyl-n-propyl)aminosulfonyl, di-(1-ethyl-n-propyl)aminosulfonyl, di-c-pentylaminosulfonyl, di-(1-methyl-c-butyl)aminosulfonyl, di-(2-methyl-c-butyl)aminosulfonyl, di-(3-methyl-c-butyl)aminosulfonyl, di-(1,2-dimethyl-c-propyl)aminosulfonyl, di-(2,3-dimethyl-c-propyl)aminosulfonyl, di-(1-ethyl-c-propyl)aminosulfonyl, di-(2-ethyl-c-propyl)aminosulfonyl, di-n-hexylaminosulfonyl, di-(1-methyl-n-pentyl)aminosulfonyl, di-(2-methyl-n-pentyl)aminosulfonyl, di-(3-methyl-n-pentyl)aminosulfonyl, di-(4-methyl-n-pentyl)aminosulfonyl, di-(1,1-dimethyl-n-butyl)aminosulfonyl, di-(1,2-dimethyl-n-butyl)aminosulfonyl, di-(1,3-dimethyl-n-butyl)aminosulfonyl, di-(2,2-dimethyl-n-butyl)aminosulfonyl, di-(2,3-dimethyl-n-butyl)aminosulfonyl, di-(3,3-dimethyl-n-butyl)aminosulfonyl, di-(1-ethyl-n-butyl)aminosulfonyl, di-(2-ethyl-n-butyl)aminosulfonyl, di-(1,1,2-trimethyl-n-propyl)aminosulfonyl, di-(1,2,2-trimethyl-n-propyl)aminosulfonyl, di-(1-ethyl-1-methyl-n-propyl)aminosulfonyl, di-(1-ethyl-2-methyl-n-propyl)aminosulfonyl, di-c-hexylaminosulfonyl, di-(1-methyl-c-pentyl)aminosulfonyl, di-(2-methyl-c-pentyl)aminosulfonyl, di-(3-methyl-c-pentyl)aminosulfonyl, di-(1-ethyl-c-butyl)aminosulfonyl, di-(2-ethyl-c-butyl)aminosulfonyl, di-(3-ethyl-c-butyl)aminosulfonyl, di-(1,2-dimethyl-c-butyl)aminosulfonyl, di-(1,3-dimethyl-c-butyl)aminosulfonyl, di-(2,2-dimethyl-c-butyl)aminosulfonyl, di-(2,3-dimethyl-c-butyl)aminosulfonyl, di-(2,4-dimethyl-c-butyl)aminosulfonyl, di-(3,3-dimethyl-c-butyl)aminosulfonyl, di-(1-n-propyl-c-propyl)aminosulfonyl, di-(2-n-propyl-c-propyl)aminosulfonyl, di-(1-i-propyl-c-propyl)aminosulfonyl, di-(2-i-propyl-c-propyl)aminosulfonyl, di-(1,2,2-trimethyl-c-propyl)aminosulfonyl, di-(1,2,3-trimethyl-c-propyl)aminosulfonyl, di-(2,2,3-trimethyl-c-propyl)aminosulfonyl, di-(1-ethyl-2-methyl-c-propyl)aminosulfonyl, di-(2-ethyl-1-methyl-c-propyl)aminosulfonyl, di-(2-ethyl-2-methyl-c-propyl)aminosulfonyl, di-(2-ethyl-3-methyl-c-propyl)aminosulfonyl, di-(1-methyl-1-ethyl-n-pentyl)aminosulfonyl, di-(1-heptyl)aminosulfonyl, di-(2-heptyl)aminosulfonyl, di-(1-ethyl-1,2-dimethyl-n-propyl)aminosulfonyl, di-(1-ethyl-2,2-dimethyl-n-propyl)aminosulfonyl, di-(1-octyl)aminosulfonyl, di-(3-octyl)aminosulfonyl, di-(4-methyl-3-n-heptyl)aminosulfonyl, di-(6-methyl-2-n-heptyl)aminosulfonyl, di-(2-propyl-1-n-heptyl)aminosulfonyl, di-(2,4,4-trimethyl-1-n-pentyl)aminosulfonyl, di-(1-nonyl)aminosulfonyl, di-(2-nonyl)aminosulfonyl, di-(2,6-dimethyl-4-n-heptyl)aminosulfonyl, di-(3-ethyl-2,2-dimethyl-3-n-pentyl)aminosulfonyl, di-(3,5,5-trimethyl-1-n-hexyl)aminosulfonyl, di-(1-decyl)aminosulfonyl, di-(2-decyl)aminosulfonyl, di-(4-decyl)aminosulfonyl, di-(3,7-dimethyl-1-n-octyl)aminosulfonyl, di-(3,7-dimethyl-3-n-octyl)aminosulfonyl or the like may be mentioned.

An asymmetric di-C₁₋₁₀ alkylaminosulfonyl group may be linear, branched or a C₃₋₁₀ cycloalkylaminosulfonyl group, and as specific examples, (methyl, ethyl)aminosulfonyl, (methyl, n-propyl)aminosulfonyl, (methyl, i-propyl)aminosulfonyl, (methyl, c-propyl)aminosulfonyl, (methyl, n-butyl)aminosulfonyl, (methyl, i-butyl)aminosulfonyl, (methyl, s-butyl)aminosulfonyl, (methyl, t-butyl)aminosulfonyl, (methyl, n-pentyl)aminosulfonyl, (methyl, c-pentyl)aminosulfonyl, (methyl, n-hexyl)aminosulfonyl, (methyl, c-hexyl)aminosulfonyl, (ethyl, n-propyl)aminosulfonyl, (ethyl, i-propyl)aminosulfonyl, (ethyl, c-propyl)aminosulfonyl, (ethyl, n-butyl)aminosulfonyl, (ethyl, i-butyl)aminosulfonyl, (ethyl, s-butyl)aminosulfonyl, (ethyl, t-butyl)aminosulfonyl, (ethyl, n-pentyl)aminosulfonyl, (ethyl, c-pentyl)aminosulfonyl, (ethyl, n-hexyl)aminosulfonyl, (ethyl, c-hexyl)aminosulfonyl, (n-propyl, i-propyl)aminosulfonyl, (n-propyl, c-propyl)aminosulfonyl, (n-propyl, n-butyl)aminosulfonyl, (n-propyl, i-butyl)aminosulfonyl, (n-propyl, s-butyl)aminosulfonyl, (n-propyl, t-butyl)aminosulfonyl, (n-propyl, n-pentyl)aminosulfonyl, (n-propyl, c-pentyl)aminosulfonyl, (n-propyl, n-hexyl)aminosulfonyl, (n-propyl, c-hexyl)aminosulfonyl, (i-propyl, c-propyl)aminosulfonyl, (i-propyl, n-butyl)aminosulfonyl, (i-propyl, i-butyl)aminosulfonyl, (i-propyl, s-butyl)aminosulfonyl, (i-propyl, t-butyl)aminosulfonyl, (i-propyl, n-pentyl)aminosulfonyl, (i-propyl, c-pentyl)aminosulfonyl, (i-propyl, n-hexyl)aminosulfonyl, (i-propyl, c-hexyl)aminosulfonyl, (c-propyl, n-butyl)aminosulfonyl, (c-propyl, i-butyl)aminosulfonyl, (c-propyl, s-butyl)aminosulfonyl, (c-propyl, t-butyl)aminosulfonyl, (c-propyl, n-pentyl)aminosulfonyl, (c-propyl, c-pentyl)aminosulfonyl, (c-propyl, n-hexyl)aminosulfonyl, (c-propyl, c-hexyl)aminosulfonyl, (n-butyl, i-butyl)aminosulfonyl, (n-butyl, s-butyl)aminosulfonyl, (n-butyl, t-butyl)aminosulfonyl, (n-butyl, n-pentyl)aminosulfonyl, (n-butyl, c-pentyl)aminosulfonyl, (n-butyl, n-hexyl)aminosulfonyl, (n-butyl, c-hexyl)aminosulfonyl, (i-butyl, s-butyl)aminosulfonyl, (i-butyl, t-butyl)aminosulfonyl, (i-butyl, n-pentyl)aminosulfonyl, (i-butyl, c-pentyl)aminosulfonyl, (i-butyl, n-hexyl)aminosulfonyl, (i-butyl, c-hexyl)aminosulfonyl, (s-butyl, t-butyl)aminosulfonyl, (s-butyl, n-pentyl)aminosulfonyl, (s-butyl, c-pentyl)aminosulfonyl, (s-butyl, n-hexyl)aminosulfonyl, (s-butyl, c-hexyl)aminosulfonyl, (t-butyl, n-pentyl)aminosulfonyl, (t-butyl, c-pentyl)aminosulfonyl, (t-butyl, n-hexyl)aminosulfonyl, (t-butyl, c-hexyl)aminosulfonyl, (n-pentyl, c-pentyl)aminosulfonyl, (n-pentyl, n-hexyl)aminosulfonyl, (n-pentyl, c-hexyl)aminosulfonyl, (c-pentyl, n-hexyl)aminosulfonyl, (c-pentyl, c-hexyl)aminosulfonyl, (n-hexyl, c-hexyl)aminosulfonyl, (methyl, n-heptyl)aminosulfonyl, (methyl, n-octyl)aminosulfonyl, (methyl, n-nonanyl)aminosulfonyl, (methyl, n-decyl)aminosulfonyl, (ethyl, n-heptyl)aminosulfonyl, (ethyl, n-octyl)aminosulfonyl, (ethyl, n-nonanyl)aminosulfonyl, (ethyl, n-decyl)aminosulfonyl or the like may be mentioned.

A C₂₋₁₄ arylene group is a bivalent group formed by removing a hydrogen atom from a ring-constituting atom in a C₂₋₁₄ aryl group, and as specific examples,

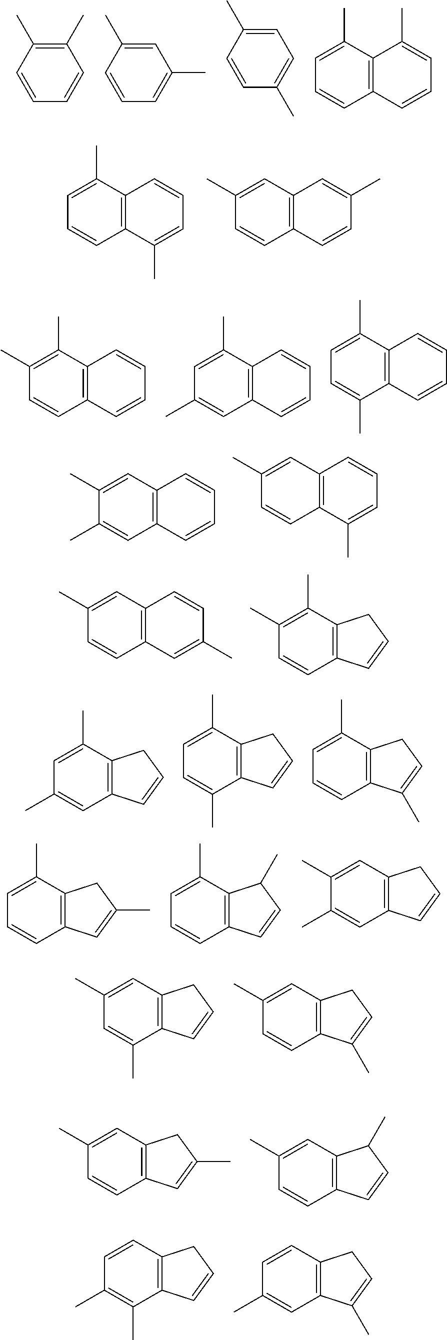

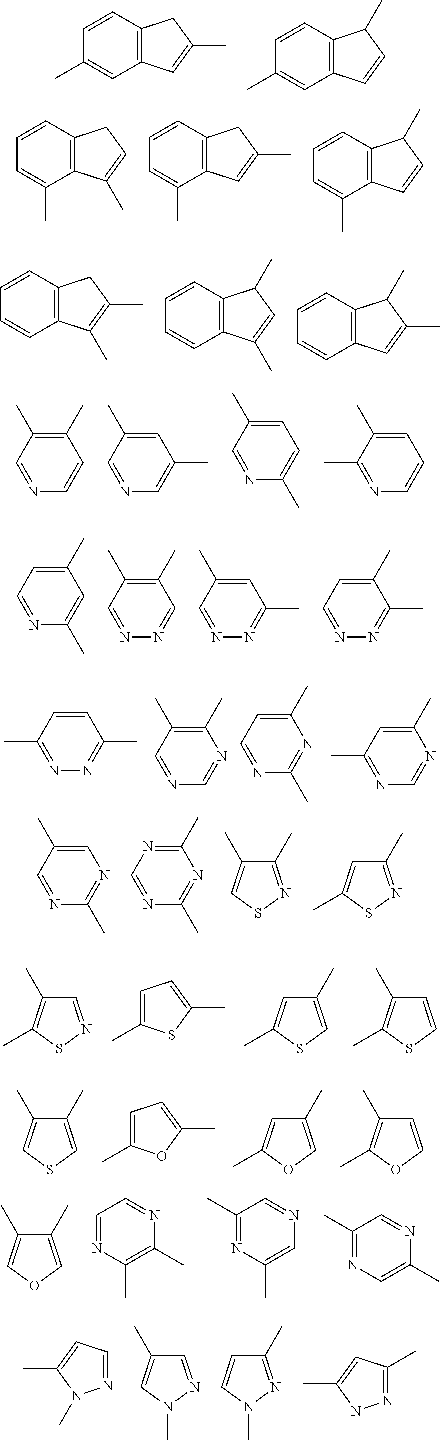

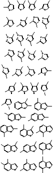

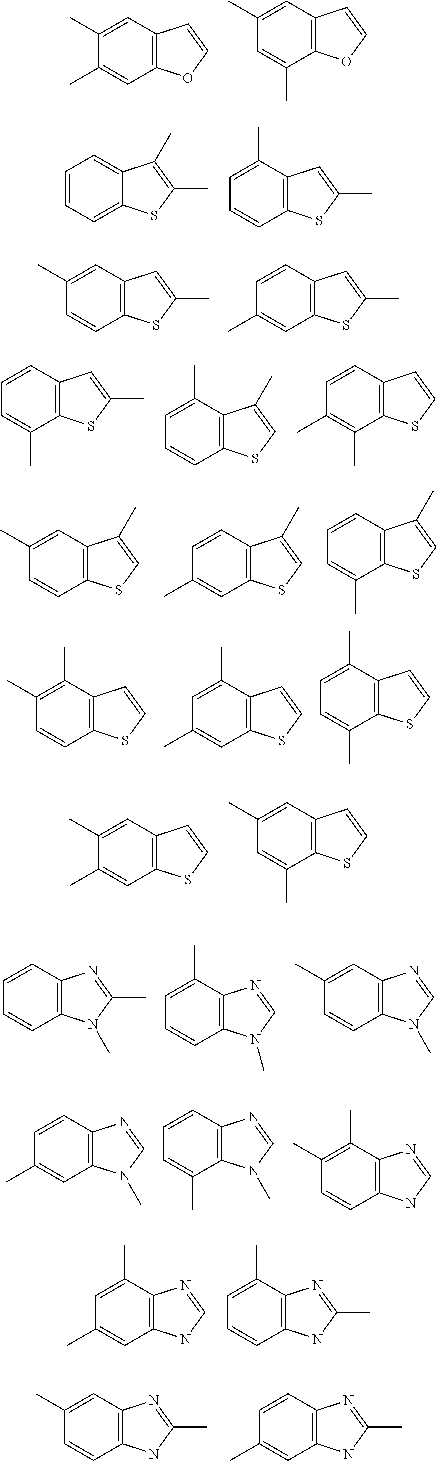

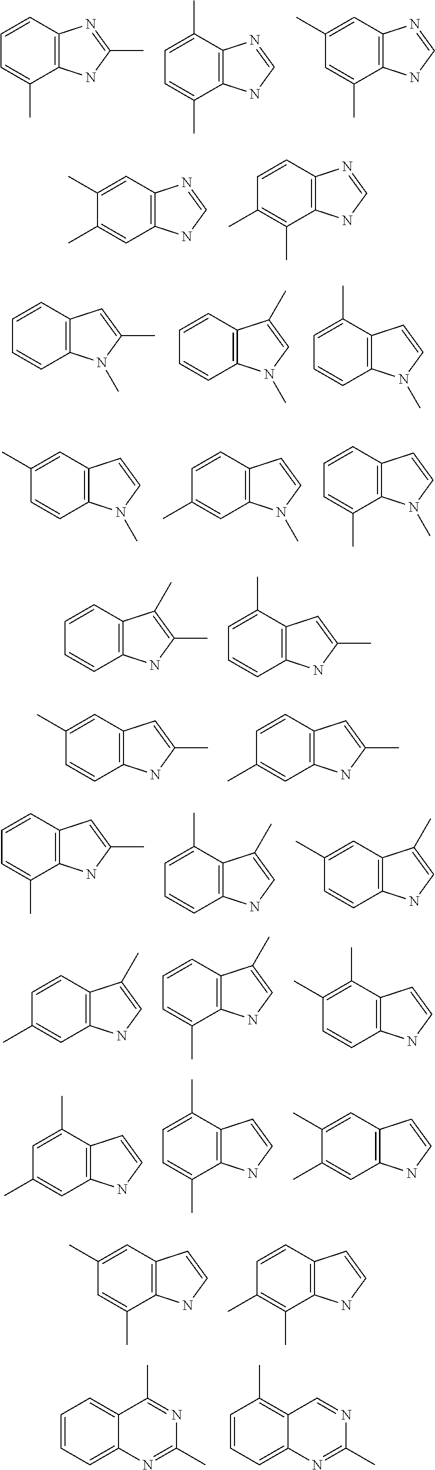

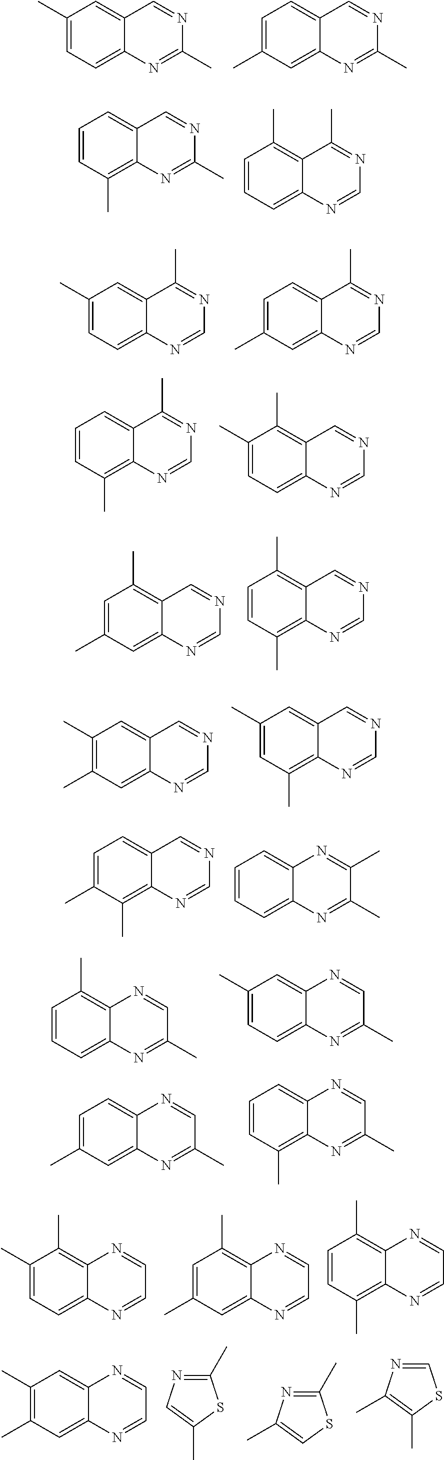

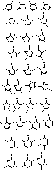

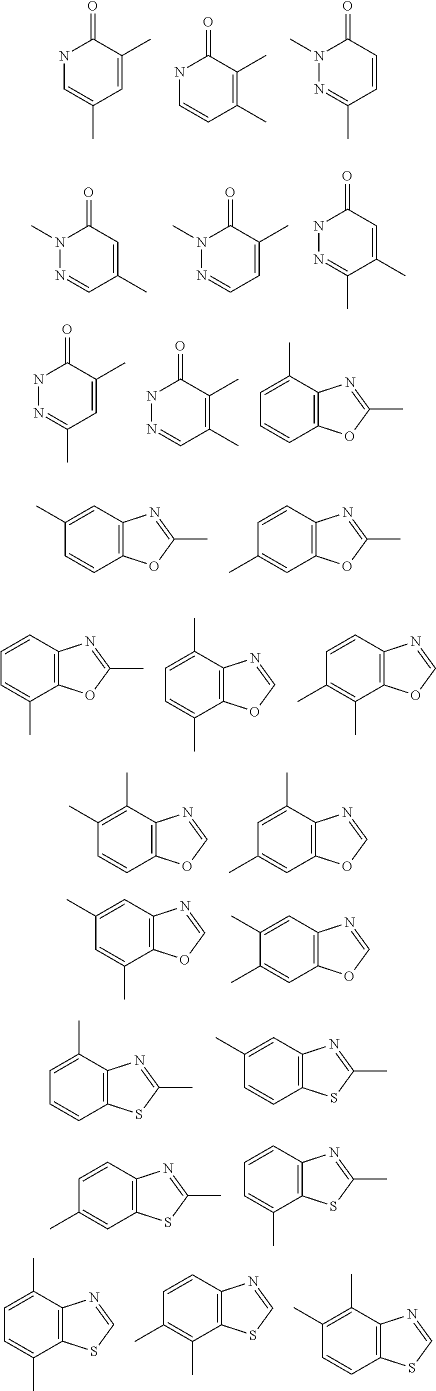

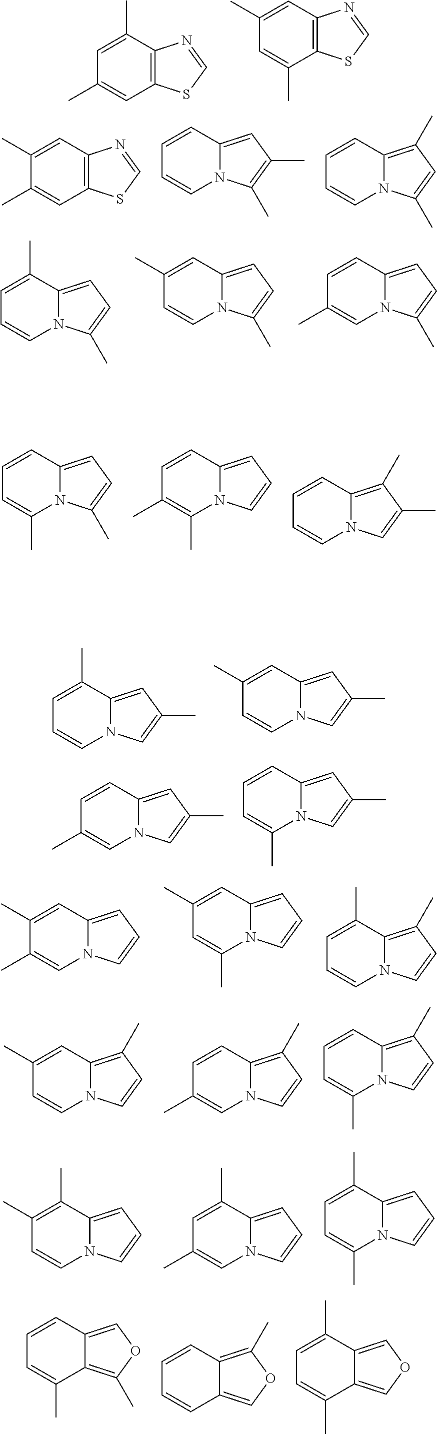

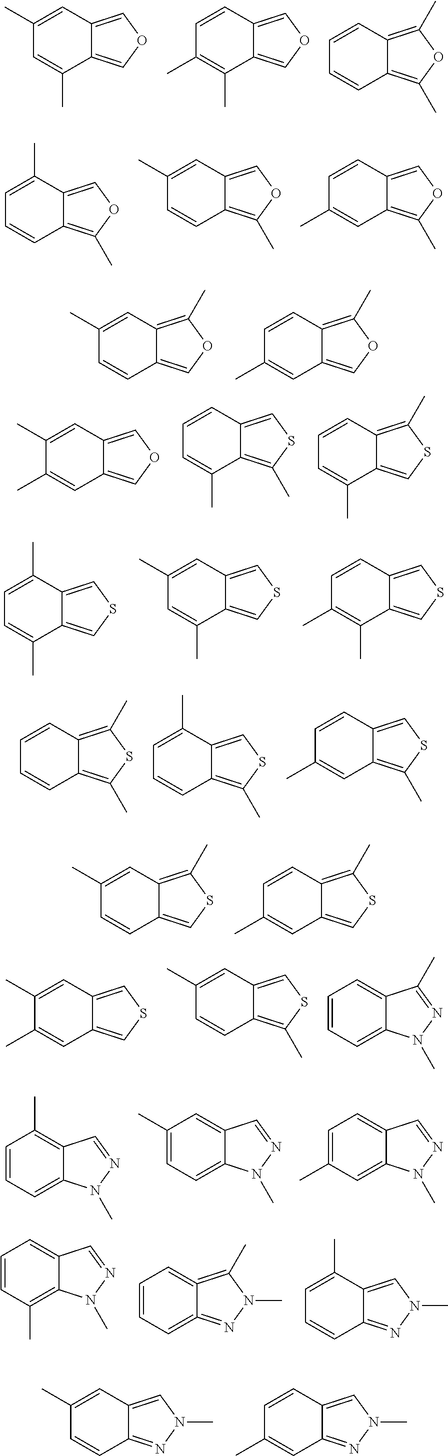

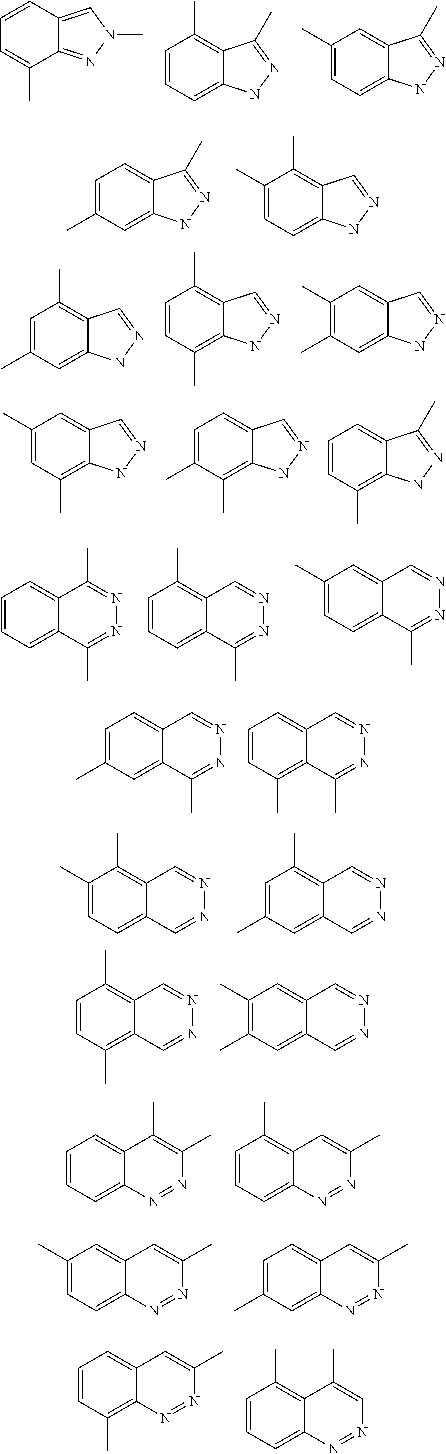

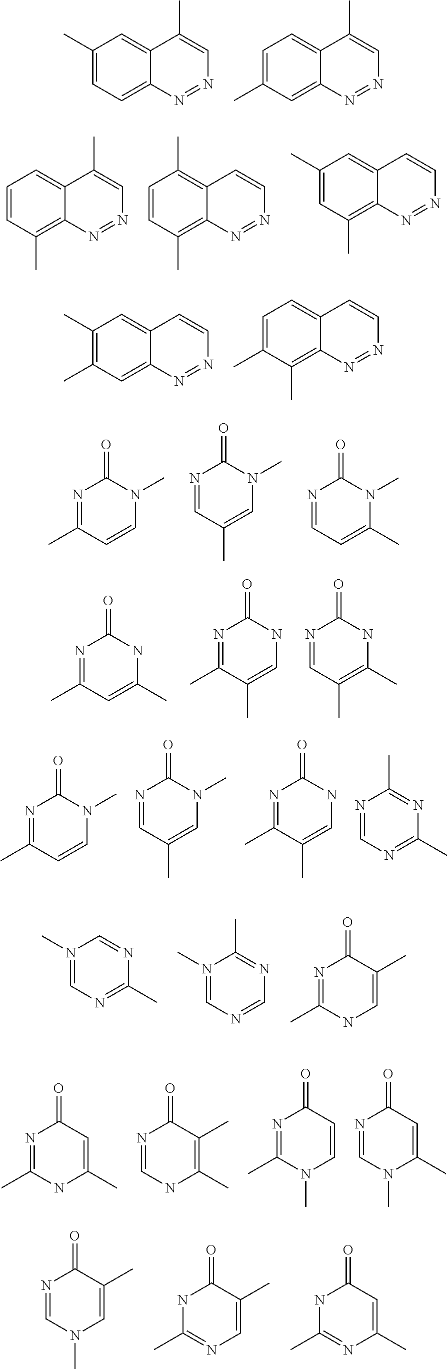

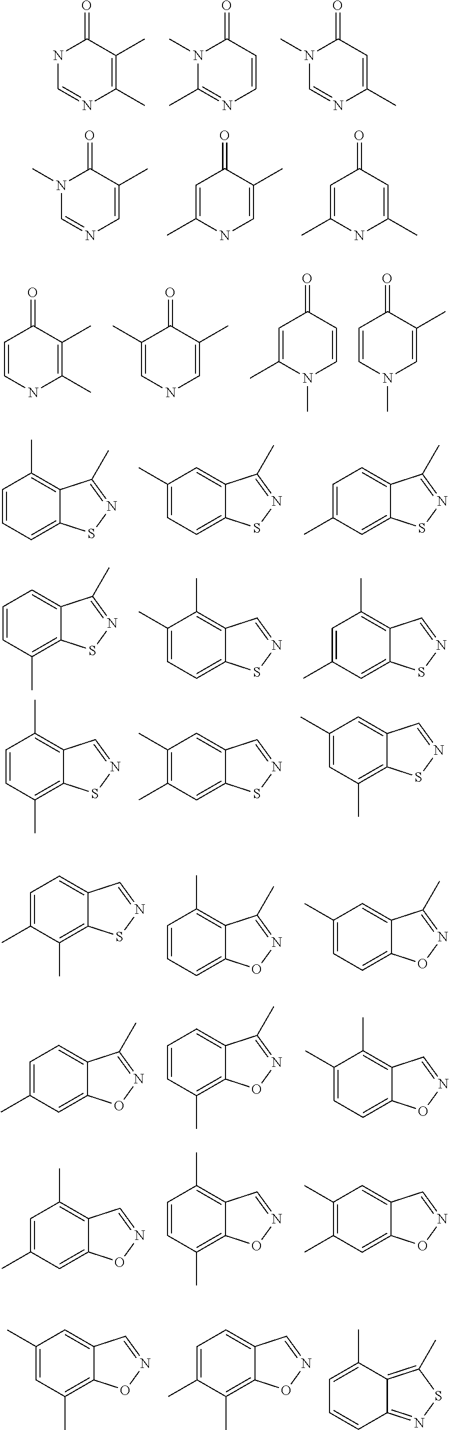

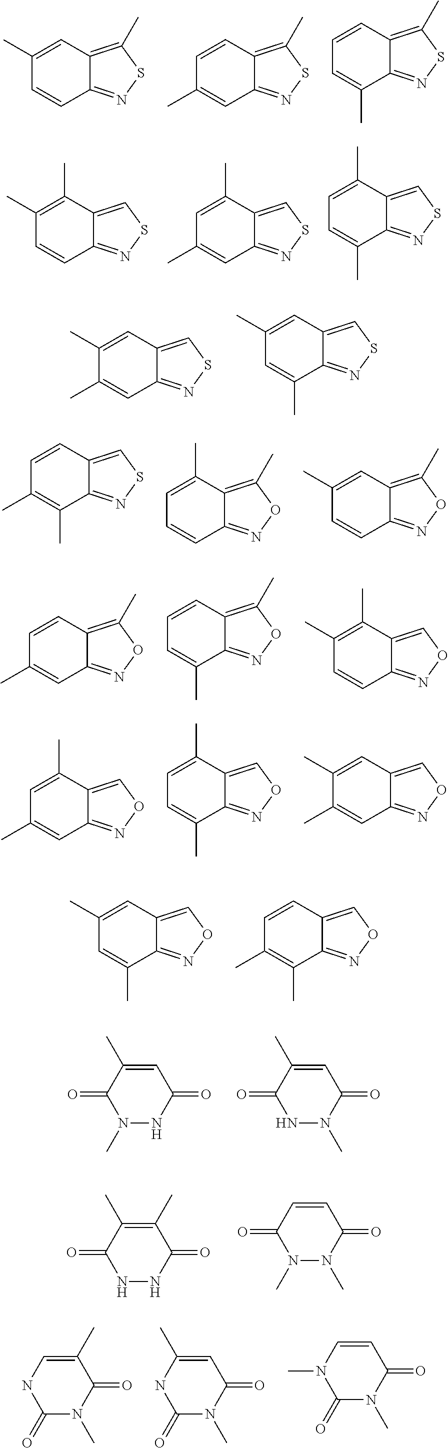

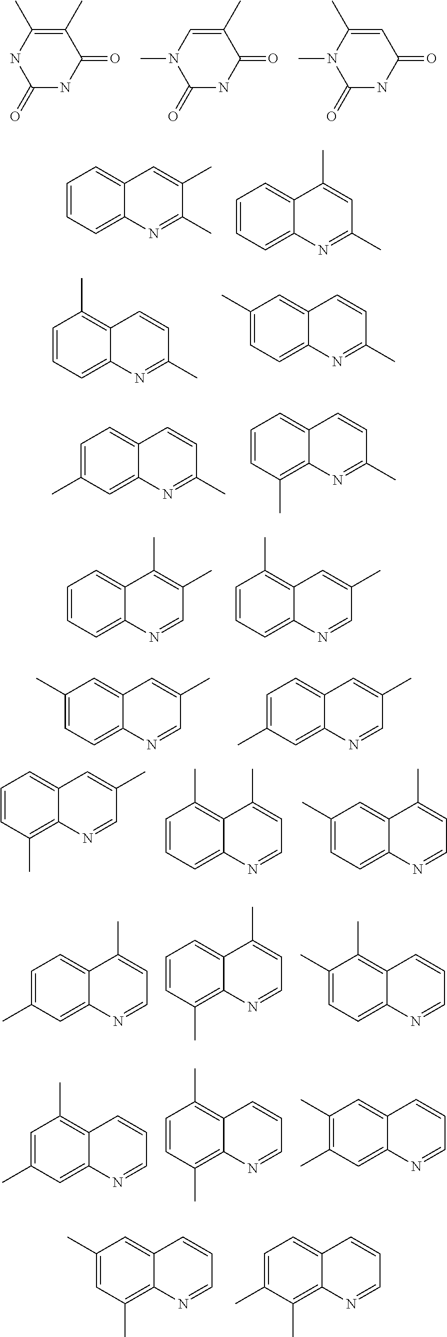

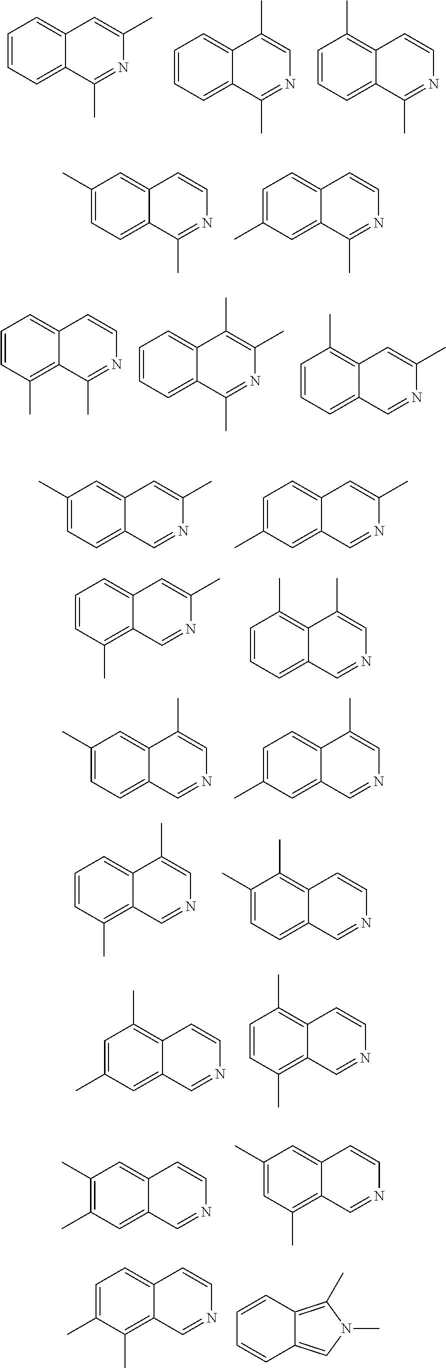

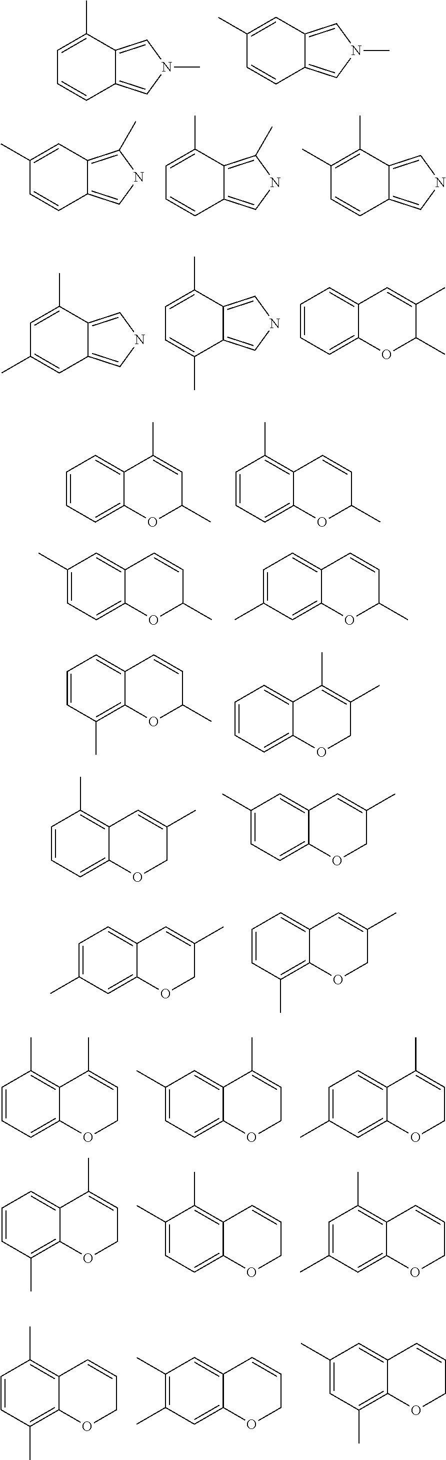

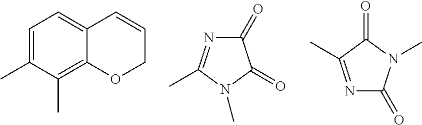

or the like may be mentioned.

A C₂₋₉ heterocyclyl group may be a monocyclic or fused bicyclic heterocyclic group containing at least one atom optionally selected from nitrogen atoms, oxygen atoms and sulfur atoms and from 2 to 9 carbon atoms, and specifically mentioned are:

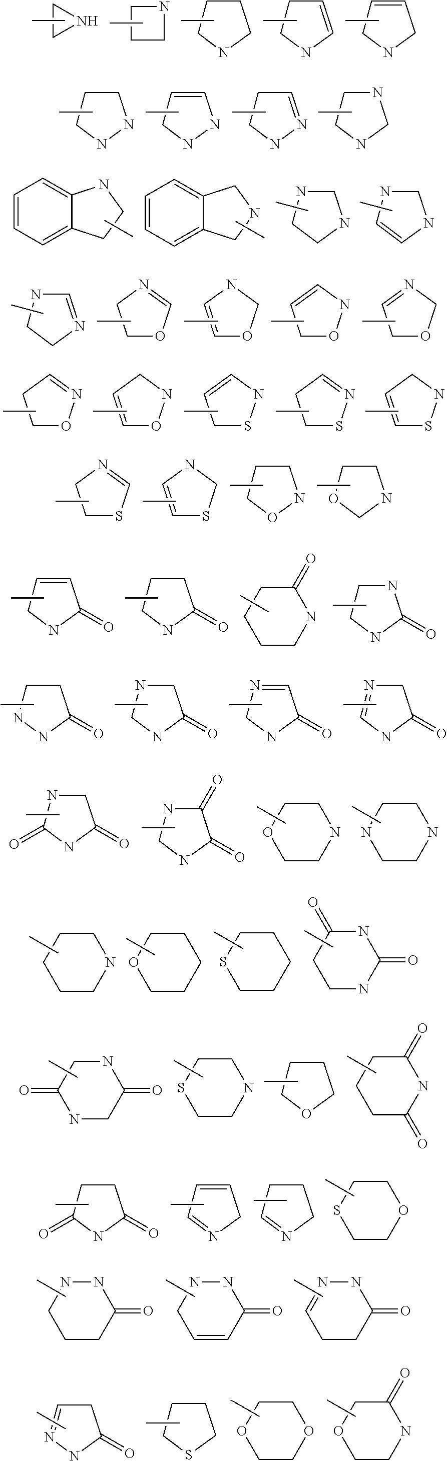

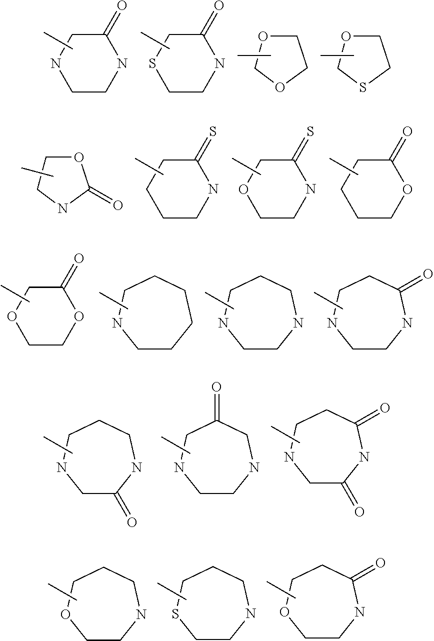

The protecting group in a protected hydroxy group, a protected amino group, a protected thiol group or an amino-protecting group may be a C₁₋₄ alkoxymethyl group (such as MOM: methoxymethyl, MEM: 2-methoxyethoxymethyl, ethoxymethyl, n-propoxymethyl, i-propoxymethyl, n-butoxymethyl, iBM: isobutyloxymethyl, BUM: t-butoxymethyl, POM: pivaloyloxymethyl, SEM: trimethylsilylethoxymethyl and the like, preferably a C₁₋₂ alkoxymethyl or the like), an aryloxymethyl (such as BOM: benzyloxymethyl, PMBM: p-methoxybenzyloxymethyl, P-AOM: p-anisyloxymethyl and the like, preferably benzyloxymethyl), a C₁₋₄ alkylaminomethyl group (such as dimethylaminomethyl), a substituted acetamidomethyl group (such as Acm: acetamidomethyl, Tacm: trimethylacetamidomethyl and the like), a substituted thiomethyl group (such as MTM: methylthiomethyl, PTM: phenylthiomethyl, Btm: benzylthiomethyl and the like), a carboxyl group, a C₁₋₇ acyl group (such as formyl, acetyl, fluoroacetyl, difluoroacetyl, trifluoroacetyl, chloroacetyl, dichloroacetyl, trichloroacetyl, propionyl, Pv: pivaloyl, tigloyl and the like), an arylcarbonyl group (such as benzoyl, p-bromobenzoyl, p-nitrobenzoyl, 2,4-dinitrobenzoyl, benzoylformyl, benzoylpropionyl, phenylpropionyl and the like), a C₁₋₄ alkoxycarbonyl group (such as methoxycarbonyl, ethoxycarbonyl, n-propoxycarbonyl, i-propoxycarbonyl, n-butoxycarbonyl, i-butoxycarbonyl, BOC: t-butoxycarbonyl, AOC: t-amyloxycarbonyl, VOC: vinyloxycarbonyl, AOC: allyloxycarbonyl, Teoc: 2-(trimethylsilyl)ethoxycarbonyl, Troc: 2,2,2-trichloroethoxycarbonyl and the like, preferably BOC and the like), an aryloxycarbonyl group (such as Z: benzyloxycarbonyl, p-nitrobenzyloxycarbonyl, MOZ: p-methoxybenzyloxycarbonyl and the like), a C₁₋₄ alkylaminocarbonyl group (such as methylcarbamoyl, Ec: ethylcarbamoyl, n-propylcarbamoyl and the like), an arylaminocarbonyl group (such as phenylcarbamoyl and the like), a trialkylsilyl group (such as TMS: trimethylsilyl, TES: triethylsilyl, TIPS: triisopropylsilyl, DEIPS: diethylisopropylsilyl, DMIPS: dimethylisopropylsilyl, DTBMS: di-t-butylmethylsilyl, IPDMS: isopropyldimethylsilyl, TBDMS: t-butyldimethylsilyl, TDS: thexyldimethylsilyl and the like, preferably t-butyldimethylsilyl and the like), a trialkylarylsilyl group (such as DPMS: diphenylmethylsilyl, TBDPS: t-butyldiphenylsilyl, TBMPS: t-butyldimethoxyphenylsilyl, TPS: triphenylsilyl and the like), an alkylsulfonyl group, (such as Ms: methanesulfonyl, ethanesulfonyl and the like) or an arylsulfonyl group (such as benzenesulfonyl, Ts: p-toluenesulfonyl, p-chlorobenzenesulfonyl, MBS: p-methoxybenzenesulfonyl, m-nitrobenzenesulfonyl, o-nitrobenzenesulfonyl, p-nitrobenzenesulfonyl, 2,4-nitrobenzenesulfonyl, iMds: 2,6-dimethoxy-4-methylbenzenesulfonyl, Mds: 2,6-dimethyl-4-methoxybenzenesulfonyl, Mtb: 2,4,6-trimethoxybenzenesulfonyl, Mte: 2,3,5,6-tetramethyl-4-methoxybenzenesulfonyl, Mtr: 2,3,6-trimethyl-4-methoxybenzenesulfonyl, Mts: 2,4,6-trimethylbenzenesulfonyl, Pme: pentamethylbenzenesulfonyl and the like).

In addition, a 1-methyl-1-methoxyethyl group, a 1-ethoxyethyl group, a 2,2,2-trichloroethyl group, a 2-trimethylsilylethoxy group, a t-butyl group, an allyl group, a benzyl group, a p-methoxybenzyl group, a 2,4-dinitrophenyl group, a p-chlorophenyl group, a p-methoxyphenyl group, a tetrahydropyranyl group, a tetrahydrofuranyl group or the like may be mentioned.

Herein, the expression “may be substituted” means that a group may have substituents in any positions of a group in each of which a substituent may be present, and that each substituent is dependent of one another.

For example, the expression “a C₁₋₃ alkoxy group which may be substituted with one or more halogen atoms” means an unsubstituted C₁₋₃ alkoxy group or an alkoxy group with a C₁₋₃ alkyl group in which optional hydrogen atom(s) may be substituted with halogen atom(s) provided that the number of halogen atoms are 2 or more, each halogen atoms may be identical to or different from one another, such as a trifluoromethoxy group, a 2,2,2-trifluoroethoxy group or a 1,1-difluoroethoxy group.

The wavy line in the formula of a group indicates a “site of bonding”.

Preferred examples of the substituents in the compounds of the present invention represented by the formula (I) are given below.

Preferred examples of R¹ are a hydrogen atom and a C₁₋₆ alkyl group which may be substituted with one or more halogen atoms, more preferred examples are a hydrogen atom and a C₁₋₃ alkyl group, and a particularly preferred example is a methyl group.

Preferred examples of R², R³, R⁴ and R⁶ are a hydrogen atom and a C₁₋₃ alkyl group (the C₁₋₃ alkyl group is unsubstituted or substituted with one or more halogen atoms), more preferred examples are a hydrogen atom and C₁₋₃ alkyl group (the C₁₋₃ alkyl group is unsubstituted), and a particularly preferred example is a hydrogen atom.

Preferred examples of R⁵ are a phenyl group which may be substituted with one or more substituents independently represented by V¹ and a C₂₋₉ heteroaryl group which may be substituted with one or more substituents independently represented by V¹, and the C₂₋₉ heteroaryl group is preferably a C₂₋₉ nitrogen-containing heteroaryl group. Specific examples of the C₂₋₉ heteroaryl group are a 2-thienyl group, a 3-thienyl group, a 2-furyl group, a 3-furyl group, a 2-pyranyl group, a 3-pyranyl group, a 4-pyranyl group, a 1-pyrrolyl group, a 2-pyrrolyl group, a 3-pyrrolyl group, a 1-imidazolyl group, a 2-imidazolyl group, a 4-imidazolyl group, a 1-pyrazolyl group, a 3-pyrazolyl group, a 4-pyrazolyl group, a 2-thiazolyl group, a 4-thiazolyl group, a 5-thiazolyl group, a 3-isothiazolyl group, a 4-isothiazolyl group, a 5-isothiazolyl group, a 1-1,2,4-triazole group, a 3-1,2,4-triazole group, a 5-1,2,4-triazole group, a 1-1,2,3-triazole group, a 4-1,2,3-triazole group, a 5-1,2,3-triazole group, a 2-oxazolyl group, a 4-oxazolyl group, a 5-oxazolyl group, a 3-isooxazolyl group, a 4-isooxazolyl group, a 5-isooxazolyl group, a 2-pyridyl group, a 3-pyridyl group, a 4-pyridyl group, a 2-pyrazinyl group, a 2-pyrimidinyl group, a 4-pyrimidinyl group, a 5-pyrimidinyl group, a 3-pyridazinyl group, a 4-pyridazinyl group, a 2-1,3,4-oxadiazolyl group, a 2-1,3,4-thiadiazolyl group, a 3-1,2,4-oxadiazolyl group, a 5-1,2,4-oxadiazolyl group, a 3-1,2,4-thiadiazolyl group, a 5-1,2,4-thiadiazolyl group, a 3-1,2,5-oxadiazolyl group and a 3-1,2,5-thiadiazolyl group.

As V¹, the formulae (V), (VI), (VII), (VIII), (IX), (X), (XI), (XII), (XIII), (XIV), (XV), (XVI), (XVII), (XVIII), (XIX), (XX), (XXI) and (XXII) may be mentioned.

More preferred examples of R⁵ are a phenyl group, a 2-thienyl group, a 3-thienyl group, a 2-pyridyl group, a 3-pyridyl group, a 4-pyridyl group, a 2-pyrazinyl group, a 2-pyrimidinyl group, a 4-pyrimidinyl group, a 5-pyrimidinyl group, a 3-pyridazinyl group, a 4-pyridazinyl group and groups obtained by substituting these groups with one or more substituents selected from the formulae (V), (VI), (VII), (VIII), (IX), (X), (XI), (XII), (XIII), (XIV), (XV), (XVI), (XVII), (XVIII), (XIX), (XX), (XXI) and (XXII).

Further more preferred examples of R⁵ are a phenyl group, a 2-pyridyl group, a 3-pyridyl group, a 4-pyridyl group, a 3-pyridazinyl group, a 4-pyridazinyl group, a 2-pyrimidinyl group, a 4-pyrimidinyl group, a 5-pyrimidinyl group, a 2-pyrazinyl group and groups obtained by substituting these groups with one or more substituents selected from the formulae (V), (VI), (VII), (VIII), (IX), (X), (XI), (XII), (XIII), (XIV), (XV), (XVI), (XVII), (XVIII), (XIX), (XX), (XXI) and (XXII).

Particularly preferred examples of R⁵ are a 4-pyridyl group, a phenyl group (the phenyl group is unsubstituted or substituted with one or more substituents selected from the formulae (V), (VI), (VII), (VIII), (IX), (X), (XI), (XII), (XIII), (XIV), (XV), (XVI), (XVII), (XVIII), (XIX), (XX), (XXI) and (XXII)) and the like.

The most preferred examples of R⁵ are a 4-pyridyl group and a phenyl group substituted with one or more substituents selected from the formulae (VII), (VIII), (XI) and (XII).

A preferred examples of R⁷ is a C₂₋₁₄ aryl group (the C₂₋₁₄ aryl group is unsubstituted or substituted with one or more substituents selected from the group consisting of C₁₋₁₀ alkyl groups (the C₁₋₁₀ alkyl groups are unsubstituted or substituted with one or more halogen atoms), halogen atoms, C₁₋₁₀ alkoxy groups and C₁₋₃ alkoxy groups (the C₁₋₃ alkoxy groups are substituted with one or more halogen atoms)).

A more preferred example of R⁷ is a phenyl group (the phenyl group is substituted with one or more substituents selected from the group consisting of C₁₋₁₀ alkyl groups (the C₁₋₁₀ alkyl groups are unsubstituted or substituted with one or more halogen atoms), halogen atoms, C₁₋₁₀ alkoxy groups and C₁₋₃ alkoxy groups (the C₁₋₃ alkoxy groups are substituted with one or more halogen atoms), and the formulae (A01), (A02), (A03), (A04), (A05), (A06), (A07), (A08), (A09), (A10), (A11), (A12), (A13), (A14) and (A15)).

Particularly preferred examples of R⁷ are a phenyl group (the phenyl group is substituted with one or more substituents selected from the group consisting of C₁₋₆ alkyl groups, C₁₋₃ alkyl groups (the C₁₋₃ alkyl groups are substituted with one or more halogen atoms), halogen atoms, C₁₋₃ alkoxy groups and C₁₋₃ alkoxy groups (the C₁₋₃ alkoxy groups are substituted with one or more halogen atoms)) and the formulae (A05), (A06), (A08), (A09), (A10), (A11), (A12), (A13), (A14) and (A15).

More specific particular preferred examples are a phenyl group (the phenyl group is substituted with one or more substituents selected from the group consisting of methyl groups, t-butyl groups, halogen atoms, methoxy groups, trifluoromethyl groups and trifluoromethoxy groups) and the formulae (A11), (A13) and (A15).

Preferred examples of Ar¹ are structures represented by the formulae (IV).

A preferred example of X is OH.

A preferred example of Y is an oxygen atom.

A preferred example of Z is an oxygen atom.

n is preferably an integer of 1 or 2, more preferably an integer of 1. When n is 1, it is particularly preferred that R⁵ is a 4-pyridyl group or a phenyl group substituted with one or more substituents selected from the formulae (VII), (VIII), (XI) and (XII).

Preferred examples of the compounds of the present invention are compounds wherein Ra, Ar and Q are any of the following combinations shown in Tables 1 to 13, tautomers or pharmaceutically acceptable salts of the compounds or solvates thereof. The symbols in Tables 1 to 13 denote the following substituents.

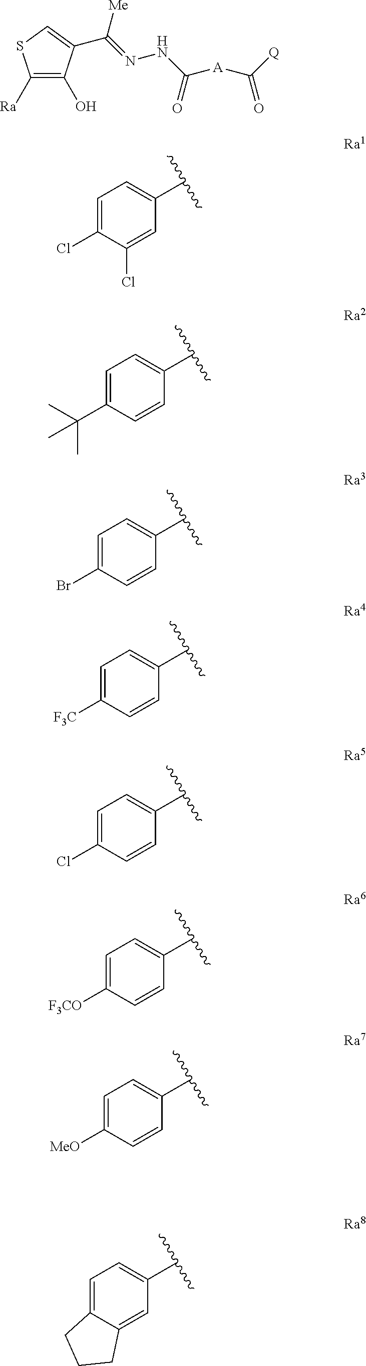

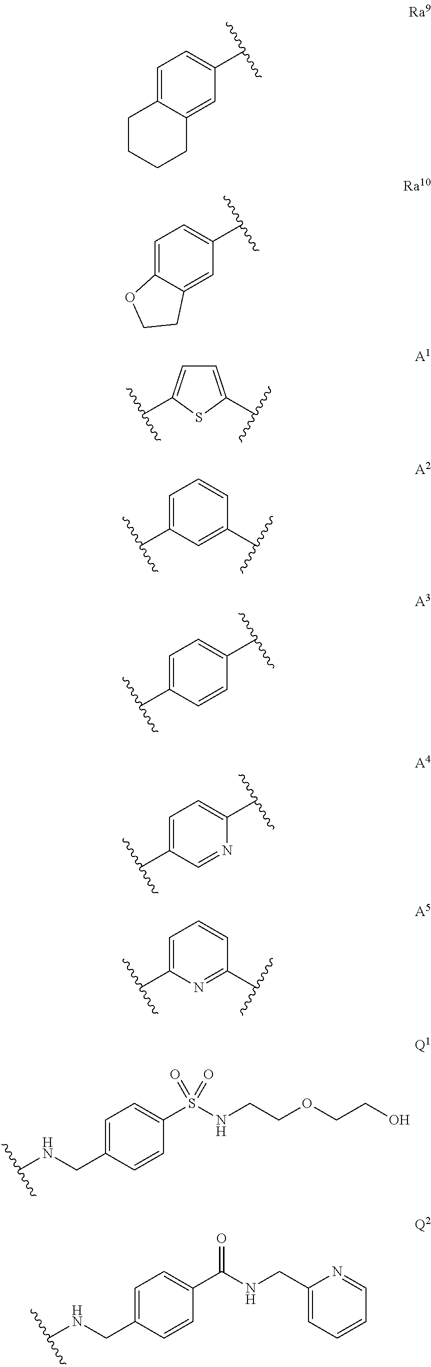

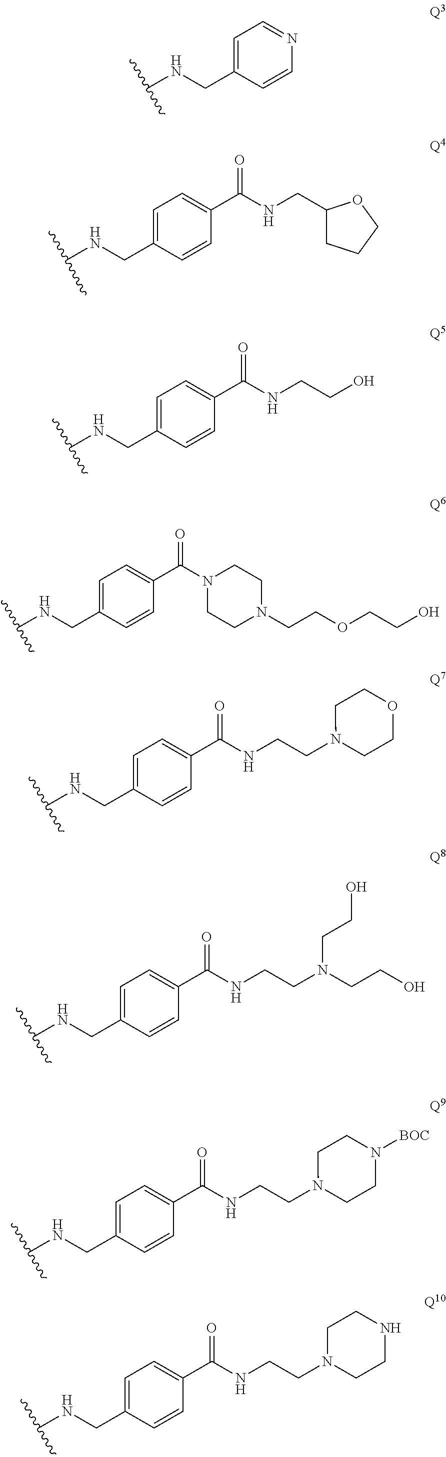

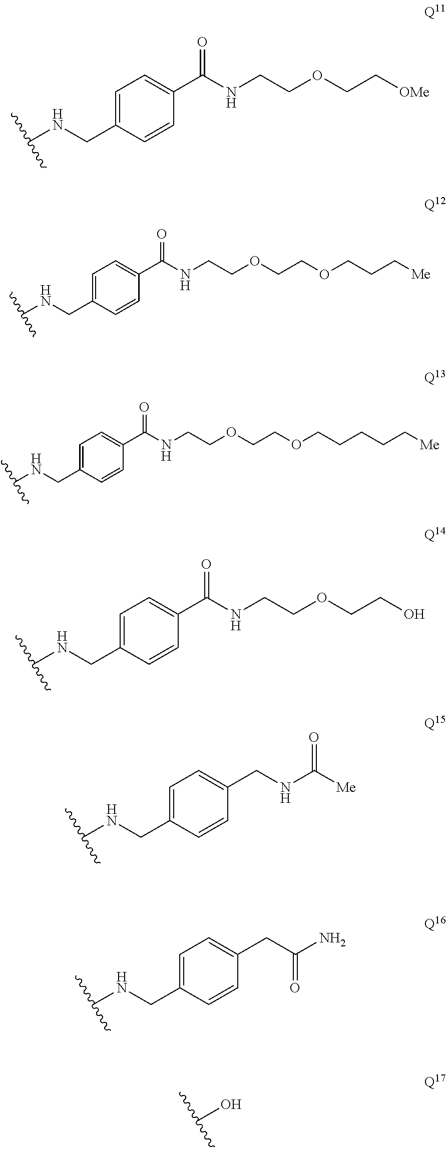

TABLE 1

| No. | Ra | A | Q |
|---|---|---|---|
| 1 | Ra¹ | A¹ | Q¹ |
| 2 | Ra¹ | A¹ | Q² |
| 3 | Ra¹ | A¹ | Q³ |
| 4 | Ra¹ | A¹ | Q⁴ |
| 5 | Ra¹ | A¹ | Q⁵ |
| 6 | Ra¹ | A¹ | Q⁶ |
| 7 | Ra¹ | A¹ | Q⁷ |
| 8 | Ra¹ | A¹ | Q⁸ |
| 9 | Ra¹ | A¹ | Q⁹ |
| 10 | Ra¹ | A¹ | Q¹⁰ |
| 11 | Ra¹ | A¹ | Q¹¹ |
| 12 | Ra¹ | A¹ | Q¹² |
| 13 | Ra¹ | A¹ | Q¹³ |
| 14 | Ra¹ | A¹ | Q¹⁴ |
| 15 | Ra¹ | A¹ | Q¹⁵ |
| 16 | Ra¹ | A¹ | Q¹⁶ |
| 17 | Ra¹ | A¹ | Q¹⁷ |
| 18 | Ra¹ | A² | Q¹ |
| 19 | Ra¹ | A² | Q² |
| 20 | Ra¹ | A² | Q³ |
| 21 | Ra¹ | A² | Q⁴ |
| 22 | Ra¹ | A² | Q⁵ |
| 23 | Ra¹ | A² | Q⁶ |
| 24 | Ra¹ | A² | Q⁷ |
| 25 | Ra¹ | A² | Q⁸ |
| 26 | Ra¹ | A² | Q⁹ |
| 27 | Ra¹ | A² | Q¹⁰ |
| 28 | Ra¹ | A² | Q¹¹ |
| 29 | Ra¹ | A² | Q¹² |
| 30 | Ra¹ | A² | Q¹³ |
| 31 | Ra¹ | A² | Q¹⁴ |
| 32 | Ra¹ | A² | Q¹⁵ |
| 33 | Ra¹ | A² | Q¹⁶ |
| 34 | Ra¹ | A² | Q¹⁷ |
| 35 | Ra¹ | A³ | Q¹ |
| 36 | Ra¹ | A³ | Q² |
| 37 | Ra¹ | A³ | Q³ |
| 38 | Ra¹ | A³ | Q⁴ |
| 39 | Ra¹ | A³ | Q⁵ |
| 40 | Ra¹ | A³ | Q⁶ |
| 41 | Ra¹ | A³ | Q⁷ |
| 42 | Ra¹ | A³ | Q⁸ |
| 43 | Ra¹ | A³ | Q⁹ |
| 44 | Ra¹ | A³ | Q¹⁰ |
| 45 | Ra¹ | A³ | Q¹¹ |
| 46 | Ra¹ | A³ | Q¹² |
| 47 | Ra¹ | A³ | Q¹³ |
| 48 | Ra¹ | A³ | Q¹⁴ |
| 49 | Ra¹ | A³ | Q¹⁵ |
| 50 | Ra¹ | A³ | Q¹⁶ |
| 51 | Ra¹ | A³ | Q¹⁷ |
| 52 | Ra¹ | A⁴ | Q¹ |
| 53 | Ra¹ | A⁴ | Q² |
| 54 | Ra¹ | A⁴ | Q³ |
| 55 | Ra¹ | A⁴ | Q⁴ |
| 56 | Ra¹ | A⁴ | Q⁵ |
| 57 | Ra¹ | A⁴ | Q⁶ |
| 58 | Ra¹ | A⁴ | Q⁷ |
| 59 | Ra¹ | A⁴ | Q⁸ |
| 60 | Ra¹ | A⁴ | Q⁹ |
| 61 | Ra¹ | A⁴ | Q¹⁰ |
| 62 | Ra¹ | A⁴ | Q¹¹ |
| 63 | Ra¹ | A⁴ | Q¹² |
| 64 | Ra¹ | A⁴ | Q¹³ |
| 65 | Ra¹ | A⁴ | Q¹⁴ |
| 66 | Ra¹ | A⁴ | Q¹⁵ |
| 67 | Ra¹ | A⁴ | Q¹⁶ |
| 68 | Ra¹ | A⁴ | Q¹⁷ |

TABLE 2

| No. | Ra | A | Q |
|---|---|---|---|
| 69 | Ra¹ | A⁵ | Q¹ |
| 70 | Ra¹ | A⁵ | Q² |
| 71 | Ra¹ | A⁵ | Q³ |
| 72 | Ra¹ | A⁵ | Q⁴ |
| 73 | Ra¹ | A⁵ | Q⁵ |
| 74 | Ra¹ | A⁵ | Q⁶ |
| 75 | Ra¹ | A⁵ | Q⁷ |
| 76 | Ra¹ | A⁵ | Q⁸ |
| 77 | Ra¹ | A⁵ | Q⁹ |
| 78 | Ra¹ | A⁵ | Q¹⁰ |
| 79 | Ra¹ | A⁵ | Q¹¹ |
| 80 | Ra¹ | A⁵ | Q¹² |
| 81 | Ra¹ | A⁵ | Q¹³ |
| 82 | Ra¹ | A⁵ | Q¹⁴ |
| 83 | Ra¹ | A⁵ | Q¹⁵ |
| 84 | Ra¹ | A⁵ | Q¹⁶ |
| 85 | Ra¹ | A⁵ | Q¹⁷ |
| 86 | Ra² | A¹ | Q¹ |
| 87 | Ra² | A¹ | Q² |
| 88 | Ra² | A¹ | Q³ |
| 89 | Ra² | A¹ | Q⁴ |
| 90 | Ra² | A¹ | Q⁵ |
| 91 | Ra² | A¹ | Q⁶ |
| 92 | Ra² | A¹ | Q⁷ |
| 93 | Ra² | A¹ | Q⁸ |
| 94 | Ra² | A¹ | Q⁹ |
| 95 | Ra² | A¹ | Q¹⁰ |
| 96 | Ra² | A¹ | Q¹¹ |
| 97 | Ra² | A¹ | Q¹² |
| 98 | Ra² | A¹ | Q¹³ |
| 99 | Ra² | A¹ | Q¹⁴ |
| 100 | Ra² | A¹ | Q¹⁵ |
| 101 | Ra² | A¹ | Q¹⁶ |
| 102 | Ra² | A¹ | Q¹⁷ |
| 103 | Ra² | A² | Q¹ |
| 104 | Ra² | A² | Q² |
| 105 | Ra² | A² | Q³ |
| 106 | Ra² | A² | Q⁴ |
| 107 | Ra² | A² | Q⁵ |
| 108 | Ra² | A² | Q⁶ |
| 109 | Ra² | A² | Q⁷ |
| 110 | Ra² | A² | Q⁸ |
| 111 | Ra² | A² | Q⁹ |
| 112 | Ra² | A² | Q¹⁰ |
| 113 | Ra² | A² | Q¹¹ |
| 114 | Ra² | A² | Q¹² |
| 115 | Ra² | A² | Q¹³ |
| 116 | Ra² | A² | Q¹⁴ |
| 117 | Ra² | A² | Q¹⁵ |
| 118 | Ra² | A² | Q¹⁶ |
| 119 | Ra² | A² | Q¹⁷ |
| 120 | Ra² | A³ | Q¹ |
| 121 | Ra² | A³ | Q² |
| 122 | Ra² | A³ | Q³ |
| 123 | Ra² | A³ | Q⁴ |
| 124 | Ra² | A³ | Q⁵ |
| 125 | Ra² | A³ | Q⁶ |
| 126 | Ra² | A³ | Q⁷ |
| 127 | Ra² | A³ | Q⁸ |
| 128 | Ra² | A³ | Q⁹ |
| 129 | Ra² | A³ | Q¹⁰ |
| 130 | Ra² | A³ | Q¹¹ |
| 131 | Ra² | A³ | Q¹² |
| 132 | Ra² | A³ | Q¹³ |
| 133 | Ra² | A³ | Q¹⁴ |
| 134 | Ra² | A³ | Q¹⁵ |
| 135 | Ra² | A³ | Q¹⁶ |
| 136 | Ra² | A³ | Q¹⁷ |

TABLE 3

| No. | Ra | A | Q |
|---|---|---|---|
| 137 | Ra² | A⁴ | Q¹ |
| 138 | Ra² | A⁴ | Q² |
| 139 | Ra² | A⁴ | Q³ |
| 140 | Ra² | A⁴ | Q⁴ |
| 141 | Ra² | A⁴ | Q⁵ |
| 142 | Ra² | A⁴ | Q⁶ |
| 143 | Ra² | A⁴ | Q⁷ |
| 144 | Ra² | A⁴ | Q⁸ |
| 145 | Ra² | A⁴ | Q⁹ |
| 146 | Ra² | A⁴ | Q¹⁰ |
| 147 | Ra² | A⁴ | Q¹¹ |
| 148 | Ra² | A⁴ | Q¹² |
| 149 | Ra² | A⁴ | Q¹³ |
| 150 | Ra² | A⁴ | Q¹⁴ |
| 151 | Ra² | A⁴ | Q¹⁵ |
| 152 | Ra² | A⁴ | Q¹⁶ |
| 153 | Ra² | A⁴ | Q¹⁷ |
| 154 | Ra² | A⁵ | Q¹ |
| 155 | Ra² | A⁵ | Q² |
| 156 | Ra² | A⁵ | Q³ |
| 157 | Ra² | A⁵ | Q⁴ |
| 158 | Ra² | A⁵ | Q⁵ |
| 159 | Ra² | A⁵ | Q⁶ |
| 160 | Ra² | A⁵ | Q⁷ |
| 161 | Ra² | A⁵ | Q⁸ |
| 162 | Ra² | A⁵ | Q⁹ |
| 163 | Ra² | A⁵ | Q¹⁰ |
| 164 | Ra² | A⁵ | Q¹¹ |
| 165 | Ra² | A⁵ | Q¹² |
| 166 | Ra² | A⁵ | Q¹³ |
| 167 | Ra² | A⁵ | Q¹⁴ |
| 168 | Ra² | A⁵ | Q¹⁵ |
| 169 | Ra² | A⁵ | Q¹⁶ |
| 170 | Ra² | A⁵ | Q¹⁷ |
| 171 | Ra³ | A¹ | Q¹ |
| 172 | Ra³ | A¹ | Q² |
| 173 | Ra³ | A¹ | Q³ |
| 174 | Ra³ | A¹ | Q⁴ |
| 175 | Ra³ | A¹ | Q⁵ |
| 176 | Ra³ | A¹ | Q⁶ |
| 177 | Ra³ | A¹ | Q⁷ |
| 178 | Ra³ | A¹ | Q⁸ |
| 179 | Ra³ | A¹ | Q⁹ |
| 180 | Ra³ | A¹ | Q¹⁰ |
| 181 | Ra³ | A¹ | Q¹¹ |
| 182 | Ra³ | A¹ | Q¹² |
| 183 | Ra³ | A¹ | Q¹³ |
| 184 | Ra³ | A¹ | Q¹⁴ |
| 185 | Ra³ | A¹ | Q¹⁵ |
| 186 | Ra³ | A¹ | Q¹⁶ |
| 187 | Ra³ | A¹ | Q¹⁷ |
| 188 | Ra³ | A² | Q¹ |
| 189 | Ra³ | A² | Q² |
| 190 | Ra³ | A² | Q³ |
| 191 | Ra³ | A² | Q⁴ |
| 192 | Ra³ | A² | Q⁵ |
| 193 | Ra³ | A² | Q⁶ |
| 194 | Ra³ | A² | Q⁷ |
| 195 | Ra³ | A² | Q⁸ |
| 196 | Ra³ | A² | Q⁹ |
| 197 | Ra³ | A² | Q¹⁰ |
| 198 | Ra³ | A² | Q¹¹ |
| 199 | Ra³ | A² | Q¹² |
| 200 | Ra³ | A² | Q¹³ |
| 201 | Ra³ | A² | Q¹⁴ |
| 202 | Ra³ | A² | Q¹⁵ |
| 203 | Ra³ | A² | Q¹⁶ |
| 204 | Ra³ | A² | Q¹⁷ |

TABLE 4

| No. | Ra | A | Q |
|---|---|---|---|
| 205 | Ra³ | A³ | Q¹ |
| 206 | Ra³ | A³ | Q² |
| 207 | Ra³ | A³ | Q³ |
| 208 | Ra³ | A³ | Q⁴ |
| 209 | Ra³ | A³ | Q⁵ |
| 210 | Ra³ | A³ | Q⁶ |
| 211 | Ra³ | A³ | Q⁷ |
| 212 | Ra³ | A³ | Q⁸ |
| 213 | Ra³ | A³ | Q⁹ |
| 214 | Ra³ | A³ | Q¹⁰ |
| 215 | Ra³ | A³ | Q¹¹ |
| 216 | Ra³ | A³ | Q¹² |
| 217 | Ra³ | A³ | Q¹³ |
| 218 | Ra³ | A³ | Q¹⁴ |
| 219 | Ra³ | A³ | Q¹⁵ |
| 220 | Ra³ | A³ | Q¹⁶ |
| 221 | Ra³ | A³ | Q¹⁷ |
| 222 | Ra³ | A⁴ | Q¹ |
| 223 | Ra³ | A⁴ | Q² |
| 224 | Ra³ | A⁴ | Q³ |
| 225 | Ra³ | A⁴ | Q⁴ |
| 226 | Ra³ | A⁴ | Q⁵ |
| 227 | Ra³ | A⁴ | Q⁶ |
| 228 | Ra³ | A⁴ | Q⁷ |
| 229 | Ra³ | A⁴ | Q⁸ |
| 230 | Ra³ | A⁴ | Q⁹ |
| 231 | Ra³ | A⁴ | Q¹⁰ |
| 232 | Ra³ | A⁴ | Q¹¹ |
| 233 | Ra³ | A⁴ | Q¹² |
| 234 | Ra³ | A⁴ | Q¹³ |
| 235 | Ra³ | A⁴ | Q¹⁴ |
| 236 | Ra³ | A⁴ | Q¹⁵ |
| 237 | Ra³ | A⁴ | Q¹⁶ |
| 238 | Ra³ | A⁴ | Q¹⁷ |
| 239 | Ra³ | A⁵ | Q¹ |
| 240 | Ra³ | A⁵ | Q² |
| 241 | Ra³ | A⁵ | Q³ |
| 242 | Ra³ | A⁵ | Q⁴ |
| 243 | Ra³ | A⁵ | Q⁵ |
| 244 | Ra³ | A⁵ | Q⁶ |
| 245 | Ra³ | A⁵ | Q⁷ |
| 246 | Ra³ | A⁵ | Q⁸ |
| 247 | Ra³ | A⁵ | Q⁹ |
| 248 | Ra³ | A⁵ | Q¹⁰ |
| 249 | Ra³ | A⁵ | Q¹¹ |
| 250 | Ra³ | A⁵ | Q¹² |
| 251 | Ra³ | A⁵ | Q¹³ |
| 252 | Ra³ | A⁵ | Q¹⁴ |
| 253 | Ra³ | A⁵ | Q¹⁵ |
| 254 | Ra³ | A⁵ | Q¹⁶ |
| 255 | Ra³ | A⁵ | Q¹⁷ |
| 256 | Ra⁴ | A¹ | Q¹ |
| 257 | Ra⁴ | A¹ | Q² |
| 258 | Ra⁴ | A¹ | Q³ |
| 259 | Ra⁴ | A¹ | Q⁴ |
| 260 | Ra⁴ | A¹ | Q⁵ |
| 261 | Ra⁴ | A¹ | Q⁶ |
| 262 | Ra⁴ | A¹ | Q⁷ |
| 263 | Ra⁴ | A¹ | Q⁸ |
| 264 | Ra⁴ | A¹ | Q⁹ |
| 265 | Ra⁴ | A¹ | Q¹⁰ |
| 266 | Ra⁴ | A¹ | Q¹¹ |
| 267 | Ra⁴ | A¹ | Q¹² |
| 268 | Ra⁴ | A¹ | Q¹³ |
| 269 | Ra⁴ | A¹ | Q¹⁴ |
| 270 | Ra⁴ | A¹ | Q¹⁵ |
| 271 | Ra⁴ | A¹ | Q¹⁶ |
| 272 | Ra⁴ | A¹ | Q¹⁷ |

TABLE 5

| No. | Ra | A | Q |
|---|---|---|---|
| 273 | Ra⁴ | A² | Q¹ |
| 274 | Ra⁴ | A² | Q² |
| 275 | Ra⁴ | A² | Q³ |
| 276 | Ra⁴ | A² | Q⁴ |
| 277 | Ra⁴ | A² | Q⁵ |
| 278 | Ra⁴ | A² | Q⁶ |
| 279 | Ra⁴ | A² | Q⁷ |
| 280 | Ra⁴ | A² | Q⁸ |
| 281 | Ra⁴ | A² | Q⁹ |
| 282 | Ra⁴ | A² | Q¹⁰ |
| 283 | Ra⁴ | A² | Q¹¹ |
| 284 | Ra⁴ | A² | Q¹² |
| 285 | Ra⁴ | A² | Q¹³ |
| 286 | Ra⁴ | A² | Q¹⁴ |
| 287 | Ra⁴ | A² | Q¹⁵ |
| 288 | Ra⁴ | A² | Q¹⁶ |
| 289 | Ra⁴ | A² | Q¹⁷ |
| 290 | Ra⁴ | A³ | Q¹ |
| 291 | Ra⁴ | A³ | Q² |
| 292 | Ra⁴ | A³ | Q³ |
| 293 | Ra⁴ | A³ | Q⁴ |
| 294 | Ra⁴ | A³ | Q⁵ |
| 295 | Ra⁴ | A³ | Q⁶ |
| 296 | Ra⁴ | A³ | Q⁷ |
| 297 | Ra⁴ | A³ | Q⁸ |
| 298 | Ra⁴ | A³ | Q⁹ |
| 299 | Ra⁴ | A³ | Q¹⁰ |
| 300 | Ra⁴ | A³ | Q¹¹ |
| 301 | Ra⁴ | A³ | Q¹² |
| 302 | Ra⁴ | A³ | Q¹³ |
| 303 | Ra⁴ | A³ | Q¹⁴ |
| 304 | Ra⁴ | A³ | Q¹⁵ |
| 305 | Ra⁴ | A³ | Q¹⁶ |
| 306 | Ra⁴ | A³ | Q¹⁷ |
| 307 | Ra⁴ | A⁴ | Q¹ |
| 308 | Ra⁴ | A⁴ | Q² |
| 309 | Ra⁴ | A⁴ | Q³ |
| 310 | Ra⁴ | A⁴ | Q⁴ |
| 311 | Ra⁴ | A⁴ | Q⁵ |
| 312 | Ra⁴ | A⁴ | Q⁶ |
| 313 | Ra⁴ | A⁴ | Q⁷ |
| 314 | Ra⁴ | A⁴ | Q⁸ |
| 315 | Ra⁴ | A⁴ | Q⁹ |
| 316 | Ra⁴ | A⁴ | Q¹⁰ |
| 317 | Ra⁴ | A⁴ | Q¹¹ |
| 318 | Ra⁴ | A⁴ | Q¹² |
| 319 | Ra⁴ | A⁴ | Q¹³ |
| 320 | Ra⁴ | A⁴ | Q¹⁴ |
| 321 | Ra⁴ | A⁴ | Q¹⁵ |
| 322 | Ra⁴ | A⁴ | Q¹⁶ |
| 323 | Ra⁴ | A⁴ | Q¹⁷ |
| 324 | Ra⁴ | A⁵ | Q¹ |
| 325 | Ra⁴ | A⁵ | Q² |
| 326 | Ra⁴ | A⁵ | Q³ |
| 327 | Ra⁴ | A⁵ | Q⁴ |
| 328 | Ra⁴ | A⁵ | Q⁵ |
| 329 | Ra⁴ | A⁵ | Q⁶ |
| 330 | Ra⁴ | A⁵ | Q⁷ |
| 331 | Ra⁴ | A⁵ | Q⁸ |
| 332 | Ra⁴ | A⁵ | Q⁹ |
| 333 | Ra⁴ | A⁵ | Q¹⁰ |
| 334 | Ra⁴ | A⁵ | Q¹¹ |
| 335 | Ra⁴ | A⁵ | Q¹² |
| 336 | Ra⁴ | A⁵ | Q¹³ |
| 337 | Ra⁴ | A⁵ | Q¹⁴ |
| 338 | Ra⁴ | A⁵ | Q¹⁵ |
| 339 | Ra⁴ | A⁵ | Q¹⁶ |
| 340 | Ra⁴ | A⁵ | Q¹⁷ |

TABLE 6

| No. | Ra | A | Q |
|---|---|---|---|
| 341 | Ra⁵ | A¹ | Q¹ |
| 342 | Ra⁵ | A¹ | Q² |
| 343 | Ra⁵ | A¹ | Q³ |
| 344 | Ra⁵ | A¹ | Q⁴ |
| 345 | Ra⁵ | A¹ | Q⁵ |
| 346 | Ra⁵ | A¹ | Q⁶ |
| 347 | Ra⁵ | A¹ | Q⁷ |
| 348 | Ra⁵ | A¹ | Q⁸ |
| 349 | Ra⁵ | A¹ | Q⁹ |
| 350 | Ra⁵ | A¹ | Q¹⁰ |
| 351 | Ra⁵ | A¹ | Q¹¹ |
| 352 | Ra⁵ | A¹ | Q¹² |
| 353 | Ra⁵ | A¹ | Q¹³ |
| 354 | Ra⁵ | A¹ | Q¹⁴ |
| 355 | Ra⁵ | A¹ | Q¹⁵ |
| 356 | Ra⁵ | A¹ | Q¹⁶ |
| 357 | Ra⁵ | A¹ | Q¹⁷ |
| 358 | Ra⁵ | A² | Q¹ |
| 359 | Ra⁵ | A² | Q² |
| 360 | Ra⁵ | A² | Q³ |
| 361 | Ra⁵ | A² | Q⁴ |
| 362 | Ra⁵ | A² | Q⁵ |
| 363 | Ra⁵ | A² | Q⁶ |
| 364 | Ra⁵ | A² | Q⁷ |
| 365 | Ra⁵ | A² | Q⁸ |
| 366 | Ra⁵ | A² | Q⁹ |
| 367 | Ra⁵ | A² | Q¹⁰ |
| 368 | Ra⁵ | A² | Q¹¹ |
| 369 | Ra⁵ | A² | Q¹² |
| 370 | Ra⁵ | A² | Q¹³ |
| 371 | Ra⁵ | A² | Q¹⁴ |
| 372 | Ra⁵ | A² | Q¹⁵ |
| 373 | Ra⁵ | A² | Q¹⁶ |
| 374 | Ra⁵ | A² | Q¹⁷ |
| 375 | Ra⁵ | A³ | Q¹ |
| 376 | Ra⁵ | A³ | Q² |
| 377 | Ra⁵ | A³ | Q³ |
| 378 | Ra⁵ | A³ | Q⁴ |
| 379 | Ra⁵ | A³ | Q⁵ |
| 380 | Ra⁵ | A³ | Q⁶ |
| 381 | Ra⁵ | A³ | Q⁷ |
| 382 | Ra⁵ | A³ | Q⁸ |
| 383 | Ra⁵ | A³ | Q⁹ |
| 384 | Ra⁵ | A³ | Q¹⁰ |
| 385 | Ra⁵ | A³ | Q¹¹ |
| 386 | Ra⁵ | A³ | Q¹² |
| 387 | Ra⁵ | A³ | Q¹³ |
| 388 | Ra⁵ | A³ | Q¹⁴ |
| 389 | Ra⁵ | A³ | Q¹⁵ |
| 390 | Ra⁵ | A³ | Q¹⁶ |
| 391 | Ra⁵ | A³ | Q¹⁷ |
| 392 | Ra⁵ | A⁴ | Q¹ |
| 393 | Ra⁵ | A⁴ | Q² |
| 394 | Ra⁵ | A⁴ | Q³ |
| 395 | Ra⁵ | A⁴ | Q⁴ |
| 396 | Ra⁵ | A⁴ | Q⁵ |
| 397 | Ra⁵ | A⁴ | Q⁶ |
| 398 | Ra⁵ | A⁴ | Q⁷ |
| 399 | Ra⁵ | A⁴ | Q⁸ |
| 400 | Ra⁵ | A⁴ | Q⁹ |
| 401 | Ra⁵ | A⁴ | Q¹⁰ |
| 402 | Ra⁵ | A⁴ | Q¹¹ |
| 403 | Ra⁵ | A⁴ | Q¹² |
| 404 | Ra⁵ | A⁴ | Q¹³ |
| 405 | Ra⁵ | A⁴ | Q¹⁴ |
| 406 | Ra⁵ | A⁴ | Q¹⁵ |
| 407 | Ra⁵ | A⁴ | Q¹⁶ |
| 408 | Ra⁵ | A⁴ | Q¹⁷ |

TABLE 7

| No. | Ra | A | Q |
|---|---|---|---|
| 409 | Ra⁵ | A⁵ | Q¹ |
| 410 | Ra⁵ | A⁵ | Q² |
| 411 | Ra⁵ | A⁵ | Q³ |
| 412 | Ra⁵ | A⁵ | Q⁴ |
| 413 | Ra⁵ | A⁵ | Q⁵ |
| 414 | Ra⁵ | A⁵ | Q⁶ |
| 415 | Ra⁵ | A⁵ | Q⁷ |
| 416 | Ra⁵ | A⁵ | Q⁸ |
| 417 | Ra⁵ | A⁵ | Q⁹ |
| 418 | Ra⁵ | A⁵ | Q¹⁰ |
| 419 | Ra⁵ | A⁵ | Q¹¹ |
| 420 | Ra⁵ | A⁵ | Q¹² |
| 421 | Ra⁵ | A⁵ | Q¹³ |
| 422 | Ra⁵ | A⁵ | Q¹⁴ |
| 423 | Ra⁵ | A⁵ | Q¹⁵ |
| 424 | Ra⁵ | A⁵ | Q¹⁶ |
| 425 | Ra⁵ | A⁵ | Q¹⁷ |
| 426 | Ra⁶ | A¹ | Q¹ |
| 427 | Ra⁶ | A¹ | Q² |
| 428 | Ra⁶ | A¹ | Q³ |
| 429 | Ra⁶ | A¹ | Q⁴ |
| 430 | Ra⁶ | A¹ | Q⁵ |
| 431 | Ra⁶ | A¹ | Q⁶ |
| 432 | Ra⁶ | A¹ | Q⁷ |
| 433 | Ra⁶ | A¹ | Q⁸ |
| 434 | Ra⁶ | A¹ | Q⁹ |
| 435 | Ra⁶ | A¹ | Q¹⁰ |
| 436 | Ra⁶ | A¹ | Q¹¹ |
| 437 | Ra⁶ | A¹ | Q¹² |
| 438 | Ra⁶ | A¹ | Q¹³ |
| 439 | Ra⁶ | A¹ | Q¹⁴ |
| 440 | Ra⁶ | A¹ | Q¹⁵ |
| 441 | Ra⁶ | A¹ | Q¹⁶ |
| 442 | Ra⁶ | A¹ | Q¹⁷ |
| 443 | Ra⁶ | A² | Q¹ |
| 444 | Ra⁶ | A² | Q² |
| 445 | Ra⁶ | A² | Q³ |
| 446 | Ra⁶ | A² | Q⁴ |
| 447 | Ra⁶ | A² | Q⁵ |
| 448 | Ra⁶ | A² | Q⁶ |
| 449 | Ra⁶ | A² | Q⁷ |
| 450 | Ra⁶ | A² | Q⁸ |
| 451 | Ra⁶ | A² | Q⁹ |
| 452 | Ra⁶ | A² | Q¹⁰ |
| 453 | Ra⁶ | A² | Q¹¹ |
| 454 | Ra⁶ | A² | Q¹² |
| 455 | Ra⁶ | A² | Q¹³ |
| 456 | Ra⁶ | A² | Q¹⁴ |
| 457 | Ra⁶ | A² | Q¹⁵ |
| 458 | Ra⁶ | A² | Q¹⁶ |
| 459 | Ra⁶ | A² | Q¹⁷ |
| 460 | Ra⁶ | A³ | Q¹ |
| 461 | Ra⁶ | A³ | Q² |
| 462 | Ra⁶ | A³ | Q³ |
| 463 | Ra⁶ | A³ | Q⁴ |
| 464 | Ra⁶ | A³ | Q⁵ |
| 465 | Ra⁶ | A³ | Q⁶ |
| 466 | Ra⁶ | A³ | Q⁷ |
| 467 | Ra⁶ | A³ | Q⁸ |
| 468 | Ra⁶ | A³ | Q⁹ |
| 469 | Ra⁶ | A³ | Q¹⁰ |
| 470 | Ra⁶ | A³ | Q¹¹ |
| 471 | Ra⁶ | A³ | Q¹² |
| 472 | Ra⁶ | A³ | Q¹³ |
| 473 | Ra⁶ | A³ | Q¹⁴ |
| 474 | Ra⁶ | A³ | Q¹⁵ |
| 475 | Ra⁶ | A³ | Q¹⁶ |
| 476 | Ra⁶ | A³ | Q¹⁷ |

TABLE 8

| No. | Ra | A | Q |
|---|---|---|---|
| 477 | Ra⁶ | A⁴ | Q¹ |
| 478 | Ra⁶ | A⁴ | Q² |
| 479 | Ra⁶ | A⁴ | Q³ |
| 480 | Ra⁶ | A⁴ | Q⁴ |
| 481 | Ra⁶ | A⁴ | Q⁵ |
| 482 | Ra⁶ | A⁴ | Q⁶ |
| 483 | Ra⁶ | A⁴ | Q⁷ |
| 484 | Ra⁶ | A⁴ | Q⁸ |
| 485 | Ra⁶ | A⁴ | Q⁹ |
| 486 | Ra⁶ | A⁴ | Q¹⁰ |
| 487 | Ra⁶ | A⁴ | Q¹¹ |
| 488 | Ra⁶ | A⁴ | Q¹² |
| 489 | Ra⁶ | A⁴ | Q¹³ |
| 490 | Ra⁶ | A⁴ | Q¹⁴ |
| 491 | Ra⁶ | A⁴ | Q¹⁵ |
| 492 | Ra⁶ | A⁴ | Q¹⁶ |
| 493 | Ra⁶ | A⁴ | Q¹⁷ |
| 494 | Ra⁶ | A⁵ | Q¹ |
| 495 | Ra⁶ | A⁵ | Q² |
| 496 | Ra⁶ | A⁵ | Q³ |
| 497 | Ra⁶ | A⁵ | Q⁴ |
| 498 | Ra⁶ | A⁵ | Q⁵ |
| 499 | Ra⁶ | A⁵ | Q⁶ |
| 500 | Ra⁶ | A⁵ | Q⁷ |
| 501 | Ra⁶ | A⁵ | Q⁸ |
| 502 | Ra⁶ | A⁵ | Q⁹ |
| 503 | Ra⁶ | A⁵ | Q¹⁰ |
| 504 | Ra⁶ | A⁵ | Q¹¹ |
| 505 | Ra⁶ | A⁵ | Q¹² |
| 506 | Ra⁶ | A⁵ | Q¹³ |
| 507 | Ra⁶ | A⁵ | Q¹⁴ |
| 508 | Ra⁶ | A⁵ | Q¹⁵ |
| 509 | Ra⁶ | A⁵ | Q¹⁶ |
| 510 | Ra⁶ | A⁵ | Q¹⁷ |
| 511 | Ra⁷ | A¹ | Q¹ |
| 512 | Ra⁷ | A¹ | Q² |
| 513 | Ra⁷ | A¹ | Q³ |
| 514 | Ra⁷ | A¹ | Q⁴ |
| 515 | Ra⁷ | A¹ | Q⁵ |
| 516 | Ra⁷ | A¹ | Q⁶ |
| 517 | Ra⁷ | A¹ | Q⁷ |
| 518 | Ra⁷ | A¹ | Q⁸ |
| 519 | Ra⁷ | A¹ | Q⁹ |
| 520 | Ra⁷ | A¹ | Q¹⁰ |
| 521 | Ra⁷ | A¹ | Q¹¹ |
| 522 | Ra⁷ | A¹ | Q¹² |
| 523 | Ra⁷ | A¹ | Q¹³ |
| 524 | Ra⁷ | A¹ | Q¹⁴ |
| 525 | Ra⁷ | A¹ | Q¹⁵ |
| 526 | Ra⁷ | A¹ | Q¹⁶ |
| 527 | Ra⁷ | A¹ | Q¹⁷ |
| 528 | Ra⁷ | A² | Q¹ |
| 529 | Ra⁷ | A² | Q² |
| 530 | Ra⁷ | A² | Q³ |
| 531 | Ra⁷ | A² | Q⁴ |
| 532 | Ra⁷ | A² | Q⁵ |
| 533 | Ra⁷ | A² | Q⁶ |
| 534 | Ra⁷ | A² | Q⁷ |
| 535 | Ra⁷ | A² | Q⁸ |
| 536 | Ra⁷ | A² | Q⁹ |
| 537 | Ra⁷ | A² | Q¹⁰ |
| 538 | Ra⁷ | A² | Q¹¹ |
| 539 | Ra⁷ | A² | Q¹² |
| 540 | Ra⁷ | A² | Q¹³ |
| 541 | Ra⁷ | A² | Q¹⁴ |
| 542 | Ra⁷ | A² | Q¹⁵ |
| 543 | Ra⁷ | A² | Q¹⁶ |
| 544 | Ra⁷ | A² | Q¹⁷ |

TABLE 9

| No. | Ra | A | Q |
|---|---|---|---|
| 545 | Ra⁷ | A³ | Q¹ |
| 546 | Ra⁷ | A³ | Q² |
| 547 | Ra⁷ | A³ | Q³ |
| 548 | Ra⁷ | A³ | Q⁴ |
| 549 | Ra⁷ | A³ | Q⁵ |
| 550 | Ra⁷ | A³ | Q⁶ |
| 551 | Ra⁷ | A³ | Q⁷ |
| 552 | Ra⁷ | A³ | Q⁸ |
| 553 | Ra⁷ | A³ | Q⁹ |
| 554 | Ra⁷ | A³ | Q¹⁰ |
| 555 | Ra⁷ | A³ | Q¹¹ |
| 556 | Ra⁷ | A³ | Q¹² |
| 557 | Ra⁷ | A³ | Q¹³ |
| 558 | Ra⁷ | A³ | Q¹⁴ |
| 559 | Ra⁷ | A³ | Q¹⁵ |
| 560 | Ra⁷ | A³ | Q¹⁶ |
| 561 | Ra⁷ | A³ | Q¹⁷ |
| 562 | Ra⁷ | A⁴ | Q¹ |
| 563 | Ra⁷ | A⁴ | Q² |
| 564 | Ra⁷ | A⁴ | Q³ |
| 565 | Ra⁷ | A⁴ | Q⁴ |
| 566 | Ra⁷ | A⁴ | Q⁵ |
| 567 | Ra⁷ | A⁴ | Q⁶ |
| 568 | Ra⁷ | A⁴ | Q⁷ |
| 569 | Ra⁷ | A⁴ | Q⁸ |
| 570 | Ra⁷ | A⁴ | Q⁹ |
| 571 | Ra⁷ | A⁴ | Q¹⁰ |
| 572 | Ra⁷ | A⁴ | Q¹¹ |
| 573 | Ra⁷ | A⁴ | Q¹² |
| 574 | Ra⁷ | A⁴ | Q¹³ |
| 575 | Ra⁷ | A⁴ | Q¹⁴ |
| 576 | Ra⁷ | A⁴ | Q¹⁵ |
| 577 | Ra⁷ | A⁴ | Q¹⁶ |
| 578 | Ra⁷ | A⁴ | Q¹⁷ |
| 579 | Ra⁷ | A⁵ | Q¹ |
| 580 | Ra⁷ | A⁵ | Q² |
| 581 | Ra⁷ | A⁵ | Q³ |
| 582 | Ra⁷ | A⁵ | Q⁴ |
| 583 | Ra⁷ | A⁵ | Q⁵ |
| 584 | Ra⁷ | A⁵ | Q⁶ |
| 585 | Ra⁷ | A⁵ | Q⁷ |
| 586 | Ra⁷ | A⁵ | Q⁸ |
| 587 | Ra⁷ | A⁵ | Q⁹ |
| 588 | Ra⁷ | A⁵ | Q¹⁰ |
| 589 | Ra⁷ | A⁵ | Q¹¹ |
| 590 | Ra⁷ | A⁵ | Q¹² |
| 591 | Ra⁷ | A⁵ | Q¹³ |
| 592 | Ra⁷ | A⁵ | Q¹⁴ |
| 593 | Ra⁷ | A⁵ | Q¹⁵ |
| 594 | Ra⁷ | A⁵ | Q¹⁶ |
| 595 | Ra⁷ | A⁵ | Q¹⁷ |
| 596 | Ra⁸ | A¹ | Q¹ |
| 597 | Ra⁸ | A¹ | Q² |
| 598 | Ra⁸ | A¹ | Q³ |
| 599 | Ra⁸ | A¹ | Q⁴ |
| 600 | Ra⁸ | A¹ | Q⁵ |
| 601 | Ra⁸ | A¹ | Q⁶ |
| 602 | Ra⁸ | A¹ | Q⁷ |
| 603 | Ra⁸ | A¹ | Q⁸ |
| 604 | Ra⁸ | A¹ | Q⁹ |
| 605 | Ra⁸ | A¹ | Q¹⁰ |
| 606 | Ra⁸ | A¹ | Q¹¹ |
| 607 | Ra⁸ | A¹ | Q¹² |
| 608 | Ra⁸ | A¹ | Q¹³ |
| 609 | Ra⁸ | A¹ | Q¹⁴ |
| 610 | Ra⁸ | A¹ | Q¹⁵ |
| 611 | Ra⁸ | A¹ | Q¹⁶ |
| 612 | Ra⁸ | A¹ | Q¹⁷ |

TABLE 10

| No. | Ra | A | Q |
|---|---|---|---|
| 613 | Ra⁸ | A² | Q¹ |
| 614 | Ra⁸ | A² | Q² |
| 615 | Ra⁸ | A² | Q³ |
| 616 | Ra⁸ | A² | Q⁴ |
| 617 | Ra⁸ | A² | Q⁵ |
| 618 | Ra⁸ | A² | Q⁶ |
| 619 | Ra⁸ | A² | Q⁷ |
| 620 | Ra⁸ | A² | Q⁸ |
| 621 | Ra⁸ | A² | Q⁹ |
| 622 | Ra⁸ | A² | Q¹⁰ |
| 623 | Ra⁸ | A² | Q¹¹ |
| 624 | Ra⁸ | A² | Q¹² |
| 625 | Ra⁸ | A² | Q¹³ |
| 626 | Ra⁸ | A² | Q¹⁴ |
| 627 | Ra⁸ | A² | Q¹⁵ |
| 628 | Ra⁸ | A² | Q¹⁶ |
| 629 | Ra⁸ | A² | Q¹⁷ |
| 630 | Ra⁸ | A³ | Q¹ |
| 631 | Ra⁸ | A³ | Q² |
| 632 | Ra⁸ | A³ | Q³ |
| 633 | Ra⁸ | A³ | Q⁴ |
| 634 | Ra⁸ | A³ | Q⁵ |
| 635 | Ra⁸ | A³ | Q⁶ |
| 636 | Ra⁸ | A³ | Q⁷ |
| 637 | Ra⁸ | A³ | Q⁸ |
| 638 | Ra⁸ | A³ | Q⁹ |
| 639 | Ra⁸ | A³ | Q¹⁰ |
| 640 | Ra⁸ | A³ | Q¹¹ |
| 641 | Ra⁸ | A³ | Q¹² |
| 642 | Ra⁸ | A³ | Q¹³ |
| 643 | Ra⁸ | A³ | Q¹⁴ |
| 644 | Ra⁸ | A³ | Q¹⁵ |
| 645 | Ra⁸ | A³ | Q¹⁶ |
| 646 | Ra⁸ | A³ | Q¹⁷ |
| 647 | Ra⁸ | A⁴ | Q¹ |
| 648 | Ra⁸ | A⁴ | Q² |
| 649 | Ra⁸ | A⁴ | Q³ |
| 650 | Ra⁸ | A⁴ | Q⁴ |
| 651 | Ra⁸ | A⁴ | Q⁵ |
| 652 | Ra⁸ | A⁴ | Q⁶ |
| 653 | Ra⁸ | A⁴ | Q⁷ |
| 654 | Ra⁸ | A⁴ | Q⁸ |
| 655 | Ra⁸ | A⁴ | Q⁹ |
| 656 | Ra⁸ | A⁴ | Q¹⁰ |
| 657 | Ra⁸ | A⁴ | Q¹¹ |
| 658 | Ra⁸ | A⁴ | Q¹² |
| 659 | Ra⁸ | A⁴ | Q¹³ |
| 660 | Ra⁸ | A⁴ | Q¹⁴ |
| 661 | Ra⁸ | A⁴ | Q¹⁵ |
| 662 | Ra⁸ | A⁴ | Q¹⁶ |
| 663 | Ra⁸ | A⁴ | Q¹⁷ |
| 664 | Ra⁸ | A⁵ | Q¹ |
| 665 | Ra⁸ | A⁵ | Q² |
| 666 | Ra⁸ | A⁵ | Q³ |
| 667 | Ra⁸ | A⁵ | Q⁴ |
| 668 | Ra⁸ | A⁵ | Q⁵ |
| 669 | Ra⁸ | A⁵ | Q⁶ |
| 670 | Ra⁸ | A⁵ | Q⁷ |
| 671 | Ra⁸ | A⁵ | Q⁸ |
| 672 | Ra⁸ | A⁵ | Q⁹ |
| 673 | Ra⁸ | A⁵ | Q¹⁰ |
| 674 | Ra⁸ | A⁵ | Q¹¹ |
| 675 | Ra⁸ | A⁵ | Q¹² |
| 676 | Ra⁸ | A⁵ | Q¹³ |
| 677 | Ra⁸ | A⁵ | Q¹⁴ |
| 678 | Ra⁸ | A⁵ | Q¹⁵ |
| 679 | Ra⁸ | A⁵ | Q¹⁶ |
| 680 | Ra⁸ | A⁵ | Q¹⁷ |

TABLE 11

| No. | Ra | A | Q |
|---|---|---|---|
| 681 | Ra⁹ | A¹ | Q¹ |
| 682 | Ra⁹ | A¹ | Q² |
| 683 | Ra⁹ | A¹ | Q³ |
| 684 | Ra⁹ | A¹ | Q⁴ |
| 685 | Ra⁹ | A¹ | Q⁵ |
| 686 | Ra⁹ | A¹ | Q⁶ |
| 687 | Ra⁹ | A¹ | Q⁷ |
| 688 | Ra⁹ | A¹ | Q⁸ |
| 689 | Ra⁹ | A¹ | Q⁹ |
| 690 | Ra⁹ | A¹ | Q¹⁰ |
| 691 | Ra⁹ | A¹ | Q¹¹ |
| 692 | Ra⁹ | A¹ | Q¹² |
| 693 | Ra⁹ | A¹ | Q¹³ |
| 694 | Ra⁹ | A¹ | Q¹⁴ |
| 695 | Ra⁹ | A¹ | Q¹⁵ |
| 696 | Ra⁹ | A¹ | Q¹⁶ |
| 697 | Ra⁹ | A¹ | Q¹⁷ |
| 698 | Ra⁹ | A² | Q¹ |
| 699 | Ra⁹ | A² | Q² |
| 700 | Ra⁹ | A² | Q³ |
| 701 | Ra⁹ | A² | Q⁴ |
| 702 | Ra⁹ | A² | Q⁵ |
| 703 | Ra⁹ | A² | Q⁶ |
| 704 | Ra⁹ | A² | Q⁷ |
| 705 | Ra⁹ | A² | Q⁸ |
| 706 | Ra⁹ | A² | Q⁹ |
| 707 | Ra⁹ | A² | Q¹⁰ |
| 708 | Ra⁹ | A² | Q¹¹ |
| 709 | Ra⁹ | A² | Q¹² |
| 710 | Ra⁹ | A² | Q¹³ |
| 711 | Ra⁹ | A² | Q¹⁴ |
| 712 | Ra⁹ | A² | Q¹⁵ |
| 713 | Ra⁹ | A² | Q¹⁶ |
| 714 | Ra⁹ | A² | Q¹⁷ |
| 715 | Ra⁹ | A³ | Q¹ |
| 716 | Ra⁹ | A³ | Q² |
| 717 | Ra⁹ | A³ | Q³ |
| 718 | Ra⁹ | A³ | Q⁴ |
| 719 | Ra⁹ | A³ | Q⁵ |
| 720 | Ra⁹ | A³ | Q⁶ |
| 721 | Ra⁹ | A³ | Q⁷ |
| 722 | Ra⁹ | A³ | Q⁸ |
| 723 | Ra⁹ | A³ | Q⁹ |
| 724 | Ra⁹ | A³ | Q¹⁰ |
| 725 | Ra⁹ | A³ | Q¹¹ |
| 726 | Ra⁹ | A³ | Q¹² |
| 727 | Ra⁹ | A³ | Q¹³ |
| 728 | Ra⁹ | A³ | Q¹⁴ |
| 729 | Ra⁹ | A³ | Q¹⁵ |
| 730 | Ra⁹ | A³ | Q¹⁶ |
| 731 | Ra⁹ | A³ | Q¹⁷ |
| 732 | Ra⁹ | A⁴ | Q¹ |
| 733 | Ra⁹ | A⁴ | Q² |
| 734 | Ra⁹ | A⁴ | Q³ |
| 735 | Ra⁹ | A⁴ | Q⁴ |
| 736 | Ra⁹ | A⁴ | Q⁵ |
| 737 | Ra⁹ | A⁴ | Q⁶ |
| 738 | Ra⁹ | A⁴ | Q⁷ |
| 739 | Ra⁹ | A⁴ | Q⁸ |
| 740 | Ra⁹ | A⁴ | Q⁹ |
| 741 | Ra⁹ | A⁴ | Q¹⁰ |
| 742 | Ra⁹ | A⁴ | Q¹¹ |
| 743 | Ra⁹ | A⁴ | Q¹² |
| 744 | Ra⁹ | A⁴ | Q¹³ |
| 745 | Ra⁹ | A⁴ | Q¹⁴ |
| 746 | Ra⁹ | A⁴ | Q¹⁵ |
| 747 | Ra⁹ | A⁴ | Q¹⁶ |
| 748 | Ra⁹ | A⁴ | Q¹⁷ |

TABLE 12

| No. | Ra | A | Q |
|---|---|---|---|
| 749 | Ra⁹ | A⁵ | Q¹ |
| 750 | Ra⁹ | A⁵ | Q² |
| 751 | Ra⁹ | A⁵ | Q³ |
| 752 | Ra⁹ | A⁵ | Q⁴ |
| 753 | Ra⁹ | A⁵ | Q⁵ |
| 754 | Ra⁹ | A⁵ | Q⁶ |
| 755 | Ra⁹ | A⁵ | Q⁷ |
| 756 | Ra⁹ | A⁵ | Q⁸ |
| 757 | Ra⁹ | A⁵ | Q⁹ |
| 758 | Ra⁹ | A⁵ | Q¹⁰ |
| 759 | Ra⁹ | A⁵ | Q¹¹ |
| 760 | Ra⁹ | A⁵ | Q¹² |
| 761 | Ra⁹ | A⁵ | Q¹³ |
| 762 | Ra⁹ | A⁵ | Q¹⁴ |
| 763 | Ra⁹ | A⁵ | Q¹⁵ |
| 764 | Ra⁹ | A⁵ | Q¹⁶ |
| 765 | Ra⁹ | A⁵ | Q¹⁷ |
| 766 | Ra¹⁰ | A¹ | Q¹ |
| 767 | Ra¹⁰ | A¹ | Q² |
| 768 | Ra¹⁰ | A¹ | Q³ |
| 769 | Ra¹⁰ | A¹ | Q⁴ |
| 770 | Ra¹⁰ | A¹ | Q⁵ |
| 771 | Ra¹⁰ | A¹ | Q⁶ |
| 772 | Ra¹⁰ | A¹ | Q⁷ |
| 773 | Ra¹⁰ | A¹ | Q⁸ |
| 774 | Ra¹⁰ | A¹ | Q⁹ |
| 775 | Ra¹⁰ | A¹ | Q¹⁰ |
| 776 | Ra¹⁰ | A¹ | Q¹¹ |
| 777 | Ra¹⁰ | A¹ | Q¹² |
| 778 | Ra¹⁰ | A¹ | Q¹³ |
| 779 | Ra¹⁰ | A¹ | Q¹⁴ |
| 780 | Ra¹⁰ | A¹ | Q¹⁵ |
| 781 | Ra¹⁰ | A¹ | Q¹⁶ |
| 782 | Ra¹⁰ | A¹ | Q¹⁷ |
| 783 | Ra¹⁰ | A² | Q¹ |
| 784 | Ra¹⁰ | A² | Q² |
| 785 | Ra¹⁰ | A² | Q³ |
| 786 | Ra¹⁰ | A² | Q⁴ |
| 787 | Ra¹⁰ | A² | Q⁵ |
| 788 | Ra¹⁰ | A² | Q⁶ |
| 789 | Ra¹⁰ | A² | Q⁷ |
| 790 | Ra¹⁰ | A² | Q⁸ |
| 791 | Ra¹⁰ | A² | Q⁹ |
| 792 | Ra¹⁰ | A² | Q¹⁰ |
| 793 | Ra¹⁰ | A² | Q¹¹ |
| 794 | Ra¹⁰ | A² | Q¹² |
| 795 | Ra¹⁰ | A² | Q¹³ |
| 796 | Ra¹⁰ | A² | Q¹⁴ |
| 797 | Ra¹⁰ | A² | Q¹⁵ |
| 798 | Ra¹⁰ | A² | Q¹⁶ |
| 799 | Ra¹⁰ | A² | Q¹⁷ |
| 800 | Ra¹⁰ | A³ | Q¹ |
| 801 | Ra¹⁰ | A³ | Q² |
| 802 | Ra¹⁰ | A³ | Q³ |
| 803 | Ra¹⁰ | A³ | Q⁴ |
| 804 | Ra¹⁰ | A³ | Q⁵ |
| 805 | Ra¹⁰ | A³ | Q⁶ |
| 806 | Ra¹⁰ | A³ | Q⁷ |
| 807 | Ra¹⁰ | A³ | Q⁸ |
| 808 | Ra¹⁰ | A³ | Q⁹ |
| 809 | Ra¹⁰ | A³ | Q¹⁰ |
| 810 | Ra¹⁰ | A³ | Q¹¹ |
| 811 | Ra¹⁰ | A³ | Q¹² |
| 812 | Ra¹⁰ | A³ | Q¹³ |
| 813 | Ra¹⁰ | A³ | Q¹⁴ |
| 814 | Ra¹⁰ | A³ | Q¹⁵ |
| 815 | Ra¹⁰ | A³ | Q¹⁶ |
| 816 | Ra¹⁰ | A³ | Q¹⁷ |

TABLE 13

| No. | Ra | A | Q |
|---|---|---|---|
| 817 | Ra⁹ | A⁴ | Q¹ |
| 818 | Ra⁹ | A⁴ | Q² |
| 819 | Ra⁹ | A⁴ | Q³ |
| 820 | Ra⁹ | A⁴ | Q⁴ |
| 821 | Ra⁹ | A⁴ | Q⁵ |
| 822 | Ra⁹ | A⁴ | Q⁶ |
| 823 | Ra⁹ | A⁴ | Q⁷ |
| 824 | Ra⁹ | A⁴ | Q⁸ |
| 825 | Ra⁹ | A⁴ | Q⁹ |
| 826 | Ra⁹ | A⁴ | Q¹⁰ |
| 827 | Ra⁹ | A⁴ | Q¹¹ |
| 828 | Ra⁹ | A⁴ | Q¹² |
| 829 | Ra⁹ | A⁴ | Q¹³ |
| 830 | Ra⁹ | A⁴ | Q¹⁴ |
| 831 | Ra⁹ | A⁴ | Q¹⁵ |
| 832 | Ra⁹ | A⁴ | Q¹⁶ |
| 833 | Ra⁹ | A⁴ | Q¹⁷ |
| 834 | Ra¹⁰ | A⁵ | Q¹ |
| 835 | Ra¹⁰ | A⁵ | Q² |
| 836 | Ra¹⁰ | A⁵ | Q³ |
| 837 | Ra¹⁰ | A⁵ | Q⁴ |
| 838 | Ra¹⁰ | A⁵ | Q⁵ |
| 839 | Ra¹⁰ | A⁵ | Q⁶ |
| 840 | Ra¹⁰ | A⁵ | Q⁷ |
| 841 | Ra¹⁰ | A⁵ | Q⁸ |
| 842 | Ra¹⁰ | A⁵ | Q⁹ |
| 843 | Ra¹⁰ | A⁵ | Q¹⁰ |
| 844 | Ra¹⁰ | A⁵ | Q¹¹ |
| 845 | Ra¹⁰ | A⁵ | Q¹² |
| 846 | Ra¹⁰ | A⁵ | Q¹³ |
| 847 | Ra¹⁰ | A⁵ | Q¹⁴ |
| 848 | Ra¹⁰ | A⁵ | Q¹⁵ |
| 849 | Ra¹⁰ | A⁵ | Q¹⁶ |
| 850 | Ra¹⁰ | A⁵ | Q¹⁷ |

A compounds of the present invention represented by the formula (I) may be converted to a pharmaceutically acceptable salt or may be liberated from the resulting salt, if necessary. The pharmaceutically acceptable salt of the present invention may be, for example, a salt with an alkali metal (such as lithium, sodium and potassium), an alkaline earth metal (such as magnesium and calcium), ammonium, an organic base or an amino acid. It may be a salt with an inorganic acid (such as hydrochloric acid, hydrobromic acid, phosphoric acid and sulfuric acid) or an organic acid (such as acetic acid, citric acid, maleic acid, fumaric acid, benzenesulfonic acid and p-toluenesulfonic acid). A compound of the present invention represented by the formula (I) or a pharmaceutically acceptable salt thereof may be in the form of arbitrary crystals or an arbitrary hydrate, depending on the production conditions. The present invention covers these crystals, hydrates and mixtures. They may be in the form of a solvate with an organic solvent such as acetone, ethanol and tetrahydrofuran, and the present invention covers any of these forms.

In the present invention, the compounds of the present invention represented by the formula (I) may be present in the form of tautomers or geometrical isomers generated by endocyclic or exocyclic isomerization, mixtures of tautomers or geometric isomers or mixtures of thereof. When the compounds of the present invention has an asymmetric center, whether or not resulting from an isomerization, the compounds of the present invention may be in the form of resolved optical isomers or in the form of mixtures containing them in certain ratios.

The compounds which serve as prodrugs are derivatives of the present invention having chemically or metabolically degradable groups which give pharmacologically active compounds of the present invention upon solvolysis or under physiological conditions in vivo. Methods for selecting or producing appropriate prodrugs are disclosed, for example, in Design of Prodrugs (Elsevier, Amsterdam 1985). In the present invention, when the compound has a hydroxy group, acyloxy derivatives obtained by reacting the compound with appropriate acyl halides or appropriate acid anhydrides may, for example, be mentioned as prodrugs. Acyloxys particularly preferred as prodrugs include —OCOC₂H₅, —OCO(t-Bu), —OCOC₁₅H₃₁, —OCO(m-CO₂Na-Ph), —OCOCH₂CH₂CO₂Na, —OCOCH(NH₂)CH₃, —OCOCH₂N(CH₃)₂ and the like. When the compounds of the present invention have an amino group, amide derivatives obtained by reacting the compound having an amino group with appropriate acid halides or appropriate mixed acid anhydrides may, for example, be mentioned as prodrugs. Amides particularly preferred as prodrugs include —NHCO(CH₂)₂₀OCH₃, —NHCOCH(NH₂)CH₃ and the like.

The specific compound to used in the method of the present invention can be synthesized chemically by reference to Patent Documents WO2004/108683, WO2006/064957, WO2007/010954, WO2010/140685 and the like, though there are no particular restrictions.

### EXAMPLES

Now, the present invention will be described in further detail with reference to Examples. However, it should be understood that the present invention is by no means restricted by these specific Examples.

The CO₂ concentration (%) in the CO₂ incubator is expressed in the percentage of the volume of CO₂ in the atmosphere. PBS denotes phosphate buffered saline (Sigma-Aldrich Japan), and FBS denotes fetal bovine serum.

### TEST EXAMPLE 1

### Preparation of Human iPS Cell-Derived Sac-Like Structures (iPS-Sacs)

In this Example, an iPS cell line TkDA3-4 (established by Tokyo University by introducing Oct3/4, Klf4, Sox2 and c-Myc into skin cells: see: WO2009122747) was used. As the feeder cells, a mouse embryo-derived cell line C3H10T1/2, provided by BloResource center, Riken Tsukuba Institute, was used. On the day before the differentiation experiment, C3H10T1/2 cells were irradiated with 50 Gy radiation, seeded on dishes coated with 0.1% gelatin at a density of from 6 to 8×10⁵/10 cm dish and used as feeder cells.

Human iPS cells were seeded on the C3H10T1/2 cells and cultured in IMDM (Invitrogen/GIBCO) supplemented with 15% FBS (JRH BIOSCIENCES, U.S.A), 2 mM L-glutamine (Invitrogen), 100 Unit/mL Penicillin-100 μg/mL Streptmycin (Sigma), ITS supplement (10 μg/mL insulin, 5.5 mg/mL transferrin, 5 ng/mL sodium selenite) (Sigma), 50 μg/mL ascorbic acid (Sigma), 0.45 mM MTG (Sigma) and 20 ng/mL VEGF (R&D systems) and incubated in 5% CO₂ at 37° C.

After about 14 to 15 days of incubation, a number of sac-like structures (iPS-sacs) containing blood cell-like cells were observed.

### TEST EXAMPLE 2

### Induction of Megakaryocytes/Platelets from the Sac-Like Structures (iPS-sacs)

Next, the sac-like structures were physically disrupted with a 10 mL disposable pipette, and hematopoietic progenitor cells and sac-like structures were separated by using a 70 μm cell strainer. The hematopoietic progenitor cells were seeded on irradiated C3H10T1/2 cells (from 6 to 8×10⁵ cells/6-well plate) newly prepared on a E-well plate, at a density of 3×10⁴ cells/well and incubated in IMDM (Invitrogen/GIBCO) supplemented with 15% FBS (JRH BIOSCIENCES, U.S.A), 2 mM L-glutamine (Invitrogen), 100 Unit/mL Penicillin-100 μg/mL Streptmysin (Sigma), ITS supplement (10 μg/mL insulin, 5.5 mg/mL transferrin, 5 ng/mL sodium selenite) (Sigma), 50 μg/mL ascorbic acid (Sigma), 0.45 mM monothioglycerol (MTG, Sigma) and 100 ng/mL human TPO (Peprotec) or one of the following specific compounds (100 ng/mL No. 1, 30 ng/mL No. 2, 50 ng/mL No. 3, 1000 ng/mL No. 4, 100 ng/mL No. 5, 100 ng/mL No. 6, 300 ng/mL No. 7, 300 ng/mL No. 8) to induce megakaryocytes/platelets.

The names and structural formulae of the specific compounds used in this Example are given below.
Specific Compound No. 1:(E)-5-(2-{1-[5-(3,4-dichlorophenyl)-4-hydroxythiophen-3-yl]ethyliden}hydrazinecarbonyl)-N-[4-(2-hydroxyethylcarbamoyl)benzyl]thiophene-2-carboxamide
Specific Compound No. 2:(E)-5-(2-{1-[5-(4-bromophenyl)-4-hydroxythiophen-3-yl]ethylidene}hydrazinecarbonyl)-N-[4-(2-hydroxyethylcarbamoyl)benzyl]thiophene-2-carboxamide
Specific Compound No. 3:(E)-5-(2-{1-[5-(3,4-dichlorophenyl)-4-hydroxythiophen-3-yl]ethylidene}hydrazinecarbonyl)-N-(pyridin-4-ylmethyl)thiophene-2-carboxamide
Specific Compound No. 4:(E)-5-(2-{1-[5-(2,3-dihydro-1H-inden-5-yl)-4-hydroxythiophen-3-yl]ethylidene}hydrazinecarbonyl)thiophene-2-carboxylic acid
Specific Compound No. 5: potassium (E)-2-(3,4-dichlorophenyl)-4-[1-(2-{5-[(pyrazin-2-ylmethyl)carbamoyl]thiophene-2-carbonyl}hydrazono)ethyl]thiophen-3-olate
Specific Compound No. 6:(E)-5-(2-{1-[5-(4-bromophenyl)-4-hydroxythiophen-3-yl]ethylidene}hydrazinecarbonyl)-N-{4-[2-(piperazin-1-yl)ethylcarbamoyl]benzyl}thiophene-2-carboxamide
Specific Compound No. 7:(E)-N-[4-(2-amino-2-oxoethyl)benzyl]-5-(2-{1-[5-(3,4-dichlorophenyl)-4-hydroxythiophen-3-yl]ethylidene}hydrazinecarbonyl)thiophene-2-carboxamide
Specific Compound No. 8:(E)-N-(4-{2-[bis(2-hydroxyethyl)amino]ethylcarbamoyl}benzyl)-5-(2-{1-[5-(4-t-butylphenyl)-4-hydroxythiophen-3-yl]ethylidene}hydrazinecarbonyl)thiophene-2-carboxamide.

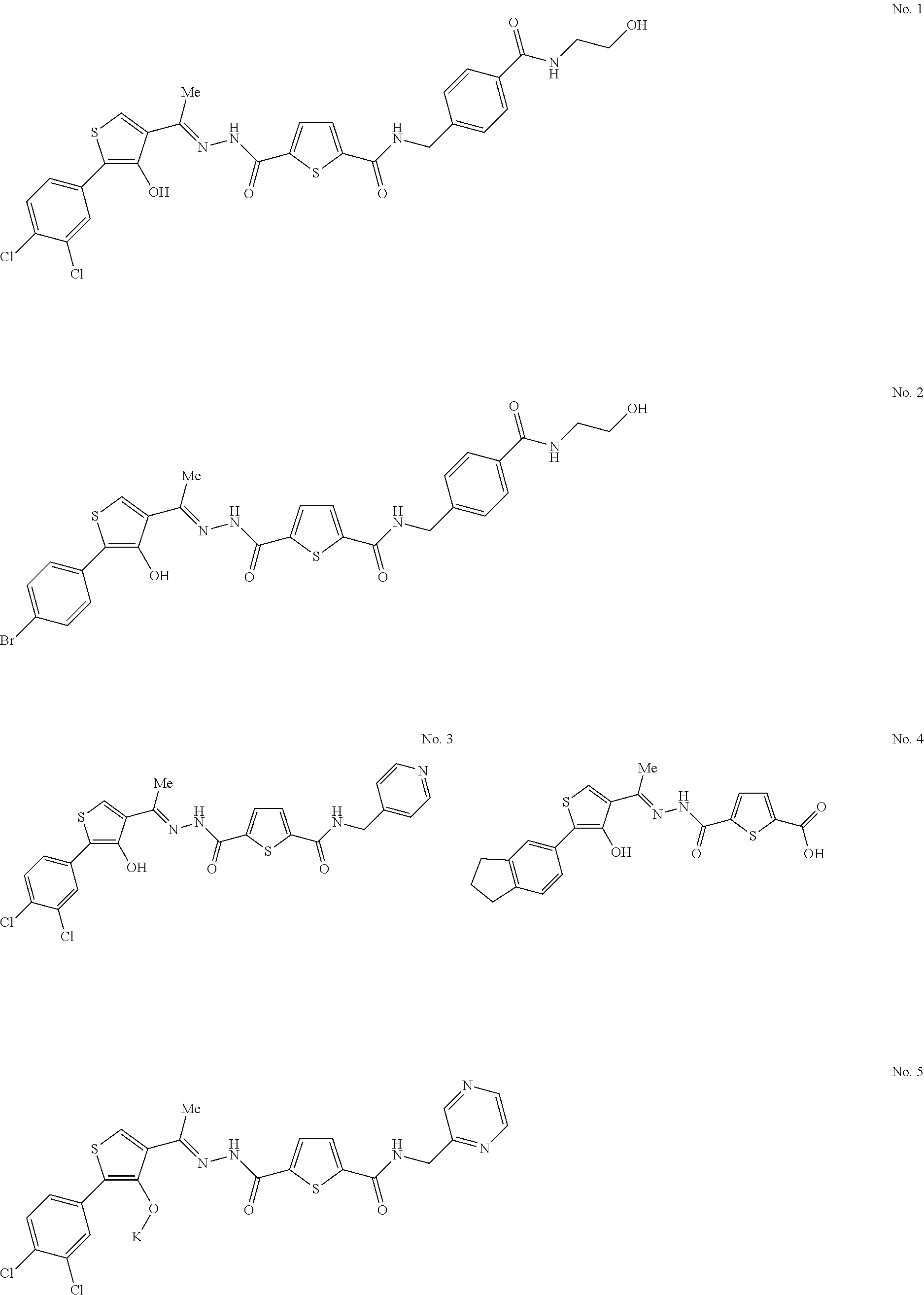

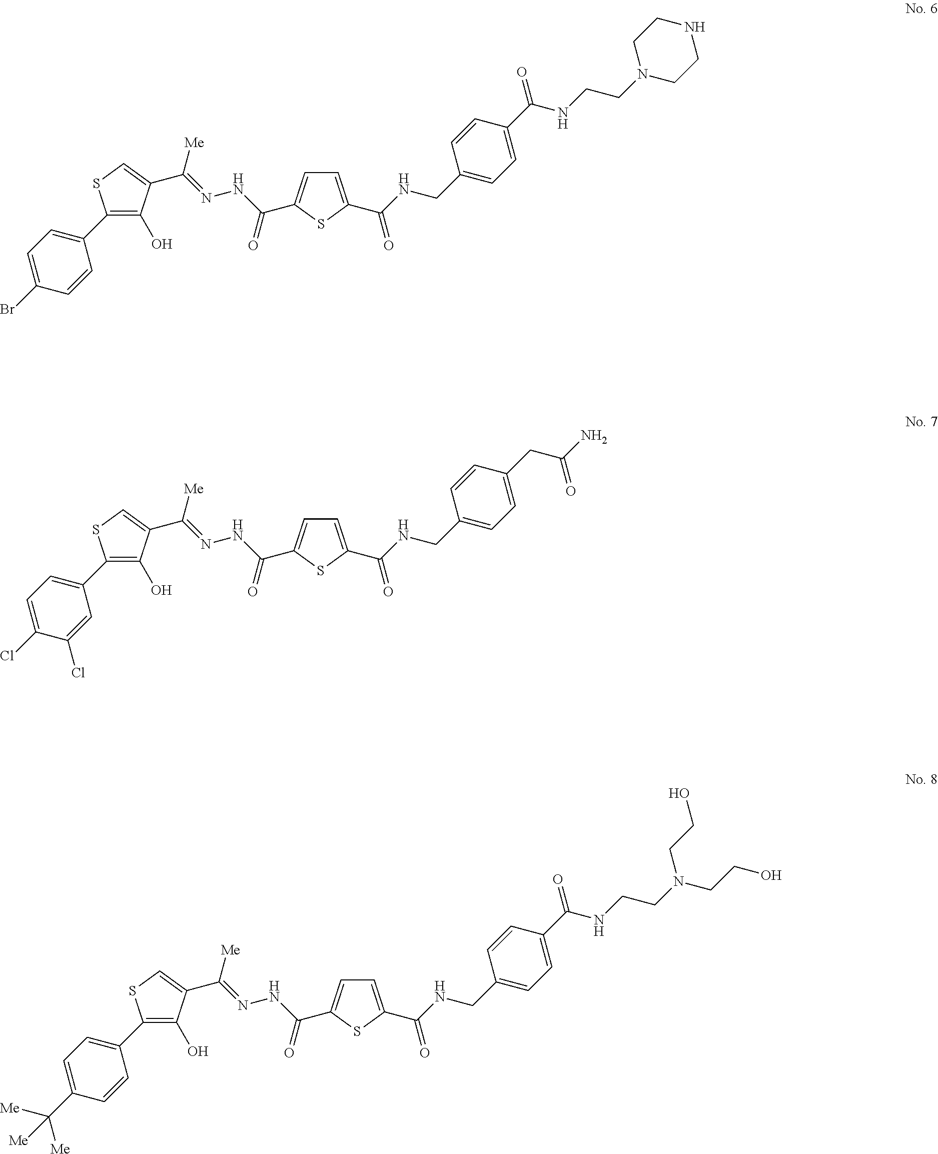 [SMILES: C/C(=N\NC(=O)c1ccc(C(=O)NCc2ccc(C(=O)NCCN3CCNCC3)cc2)s1)c1csc(-c2ccc(Br)cc2)c1O ; C/C(=N\NC(=O)c1ccc(C(=O)NCc2ccc(CC(N)=O)cc2)s1)c1csc(-c2ccc(Cl)c(Cl)c2)c1O ; C/C(=N\NC(=O)c1ccc(C(=O)NCc2ccc(C(=O)NCCN(CCO)CCO)cc2)s1)c1csc(-c2ccc(C(C)(C)C)cc2)c1O]

These compounds were synthesized by known procedures in accordance with, WO2007/010954, WO2010/140685, WO2011/049213 and the like.

### TEST EXAMPLE 3

### Megakaryocyte/Platelet Counts in Cell Cultures

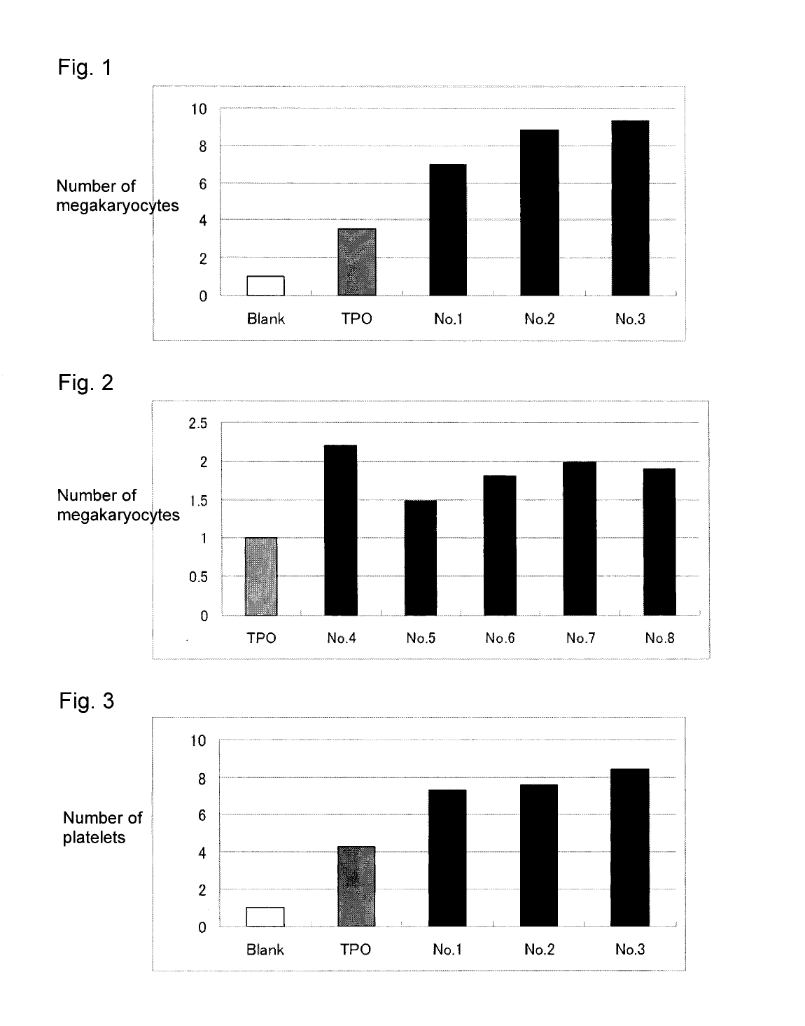

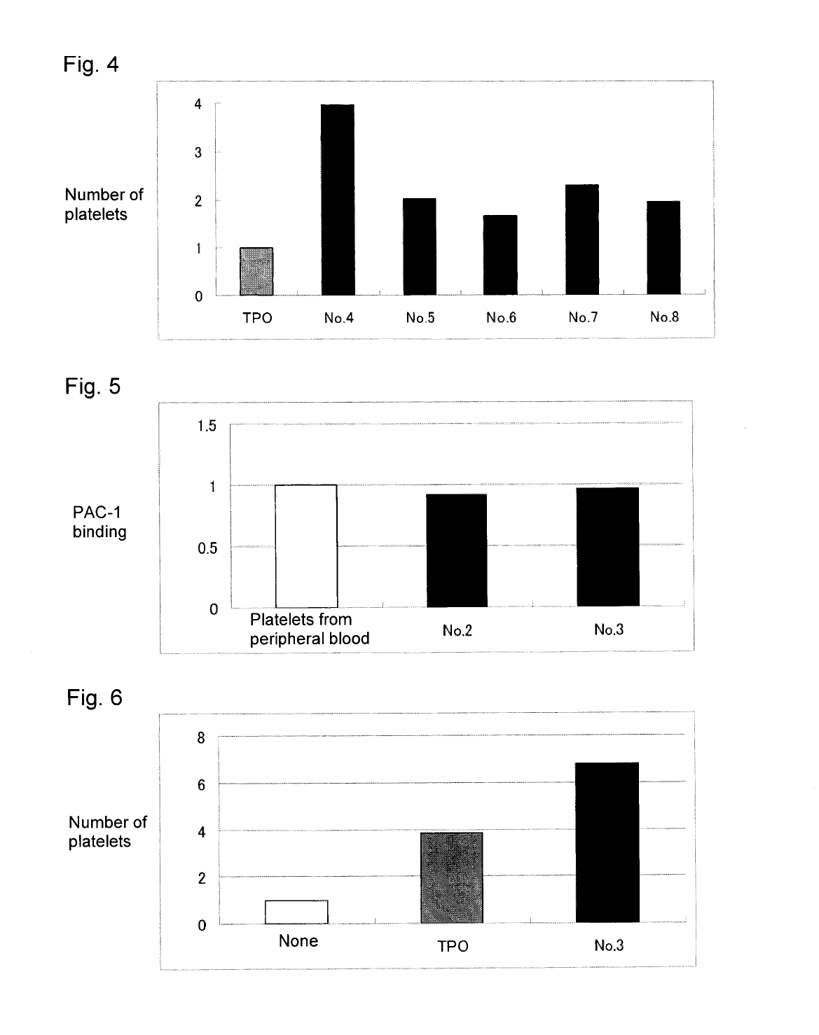

The nonadherent cells in the 23- to 24-day TkDA3-4 cultures were characterized by cell surface antigens with a fluocytometer (Becton, Dickinson and Company, BDFACSAia) after addition of 8.5 mM sodium citrate (Sigma), 6.5 mM citric acid (Sigma), 10.4 mM glucose (Sigma), anti-human CD41a antibody (Becton, Dickinson and Company) and anti-human CD42b antibody (BioLegend) in terms of final concentration. Platelets were sorted out by size with a flow cytometer and counted by using BD Trucount tubes (Becton, Dickinson and Company). Megakaryocytes were sorted by size from platelets by centrifugation (310 g, 5 minutes) and flow cytometry and counted with a hemocytometer. The megakaryocytes and platelets were positive for the cell surface antigens specific to megakaryocytes and platelets, human CD41a (integrin αIIb) and human CD42b (GPIbα) (FIGS. 1 and 2; megakaryocytes, FIGS. 3 and 4; platelets). The specific compounds of the present invention showed higher megakaryopoietic and thrombopoietic effects than TPO.

### TEST EXAMPLE 4

### Functional Analysis of the Platelets

Next, activation of integrin by platelet activators was examined. From nonadherent cells in 23- or 24-day culture of TkDA3-4 cells, nucleate cells were removed, and platelets were separated by centrifugation (400 g, 10 minutes) and treated with human anti-CD42b antibody (BioLegend), FITC-labeled PAC-1 (Becton, Dickinson and Company) and 500 μM a platelet activator, adenosine diphosphate (ADP, Sigma). 15 minutes later, the binding of PAC-1 as a platelet activation maker to palatelets was analyzed with a flow cytometer and expressed as the mean fluorescence intensity (MFI). As a result, the platelets derived from human iPS cells by using the specific compounds of the present invention showed as much activation of the integrin (binding of PAC-1 to platelets) as platelets from peripheral blood. The results demonstrate that platelets derived from iPS cells by using a specific compound of the present invention are as functional as platelets from peripheral blood.

### TEST EXAMPLE 5

### Preparation of Human ES Cell-Derived Sac-Like Structures (ES-sacs)

In this Example, an ES cell line KhES-3 (established by Kyoto University, see: Biochem Biophys Res Commun. 2006. 345: 926-932) was used. As the feeder cells, a mouse embryo-derived cell line C3H10T1/2, provided by BloResource center, Riken Tsukuba Institute, was used. On the day before the differentiation experiment, C3H10T1/2 cells were irradiated with 50 Gy radiation, seeded on dishes coated with 0.1% gelatin at a density of from 6 to 8×10⁵/10 cm dish and used as feeder cells.

Human ES cells were seeded on the C3H10T1/2 cells and cultured in IMDM (Invitrogen/GIBCO) supplemented with 15% FBS (JRH BIOSCIENCES, U.S.A), 2 mM L-glutamine (Invitrogen), 100 Unit/mL Penicillin-100 μg/mL Streptmycin (Sigma), ITS supplement (10 μg/mL insulin, 5.5 mg/mL transferrin, 5 ng/mL sodium selenite) (Sigma), 50 μg/mL ascorbic acid (Sigma), 0.45 mM MTG (Sigma) and 20 ng/mL VEGF (R&D systems) and incubated in 5% CO₂ at 37° C.

After about 14 to 15 days of incubation, a number of sac-like structures (ES-sacs) containing blood cell-like cells were observed.

### TEST EXAMPLE 6

### Induction of Megakaryocytes/Platelets from the Sac-Like Structures (ES-sacs)

Next, the sac-like structures were mechanically disrupted with a 10 mL disposable pipette, and hematopoietic progenitor cells and sac-like structures were separated by using a 70 μm cell strainer. The hematopoietic progenitor cells were seeded on irradiated C₃H₁₀T1/2 cells (from 6 to 8×10⁵ cells/6-well plate) newly prepared on a 6-well plate, at a density of 3×10⁴ cells/well and incubated in IMDM (Invitrogen/GIBCO) supplemented with 15% FBS (JRH BIOSCIENCES, U.S.A), 2 mM L-glutamine (Invitrogen), 100 Unit/mL Penicillin-100 μg/mL Streptmysin (Sigma), ITS supplement (10 μg/mL insulin, 5.5 mg/mL transferrin, 5 ng/mL sodium selenite) (Sigma), 50 μg/mL ascorbic acid (Sigma), 0.45 mM monothioglycerol (MTG, Sigma) and 100 ng/mL human TPO (Peprotec) or a specific compound used in Test Example 2 (30 ng/mL No.2, 50 ng/mL No.3) to induce megakaryocytes/platelets.

### TEST EXAMPLE 7

### Megakaryocyte/Platelet Counts in Cell Cultures

The nonadherent cells in the 23- to 24-day KhES-3 cultures were characterized by cell surface antigens after addition of 8.5 mM sodium citrate (Sigma), 6.5 mM citric acid (Sigma), 10.4 mM glucose (Sigma), human anti-CD41a antibody (Becton, Dickinson and Company) and human anti-CD42b antibody (BioLegend) in terms of final concentration. Platelets were sorted out by size with a flow cytometer and counted by using BD Trucount tubes (Becton, Dickinson and Company). Megakaryocytes were sorted by size from platelets by centrifugation (310 g, 5 minutes) and flow cytometry and counted with a hemocytometer. The megakaryocytes and platelets were positive for the cell surface antigens specific to megakaryocytes and platelets, human CD41a (integrin αIIb) and human CD42b (GPIbα) (FIG. 6; platelet counts). The specific compound of the present invention showed higher megakaryopoietic and thrombopoietic effects than TPO did.

### TEST EXAMPLE 8

### Preparation of Genetically Manipulated Hematopoietic Progenitor Cells

Hematopoietic progenitor cells were obtained from KhES-3 cell-derived sac-like structures obtained in Test Examples 5 and 6, and cells (Myc-Bmi cell line) showing enhanced proliferative capability in the presence of estradiol through enhanced expression of the oncogene c-Myc and the polycomb gene Bmi1 by using a pMX tet off vector system for regulated gene expression were obtained by using the method described in WO 2011/034073. In the absence of estradiol in the presence of doxycylcline, Myc-Bmi cells show repressed expression of c-Myc and Bmi1 and produce functional platelets, but hardly proliferate. From the Myc-Bmi cells, cells (Myc-Bmi-BCLXL cell line) which show enhanced expression of the apoptosis suppressor gene BCLXL in the absence of estradiol in the presence of doxycycline and can proliferate even in the absence of estradiol in the presence doxycycline by using an Ai-Lv tet on g vector system for regulated gene expression (Clontech) were obtained. Further, expression of p53 gene was suppressed by short hairpin (sh) RNA interference to promote polyploidization in the course of differentiation into mature megakaryocytes. Myc-Bmi-BCLXL cells were transfected with a FG12 lenti virus carrying shp53 to obtain Myc-Bmi-BCLXL cells showing repressed p53 expression (p53 KD-Myc-Bmi-BCLXL cell line). The p53 KD-Myc-Bmi-BCLXL cells were maintained by culturing on C3H10T1/2 cells inactivated by preliminary treatment with 10 μg/mL mitocycin C (Wako Pure Chemical Industries) for 0.5 to 5 hours in IMDM (Invitrogen/GIBCO) supplemented with 15% FBS (Invitrogen/GIBCO), 2m M L-glutamine-100 Unit/mL Penicillin-100 μg/mL Streptmysin (Invitrogen/GIBCO), ITS supplement (10 μg/mL insulin, 5.5 mg/mL transferrin, 5 ng/mL sodium selenite) (Invitrogen/GIBCO), 50 μg/mL ascorbic acid (Sigma), 0.45 mM monothighlycerol (MTG, Sigma), 10 μg/mL doxycycline (Clontech), 50 ng/mL SCF (R&D system) and 100 ng/mL human TPO (R&D system) in 5% CO₂ at 39° C.

### TEST EXAMPLE 9

### Induction of Megakaryocytes/Platelets from Genetically Manipulated Hematopoietic Progenitor Cells

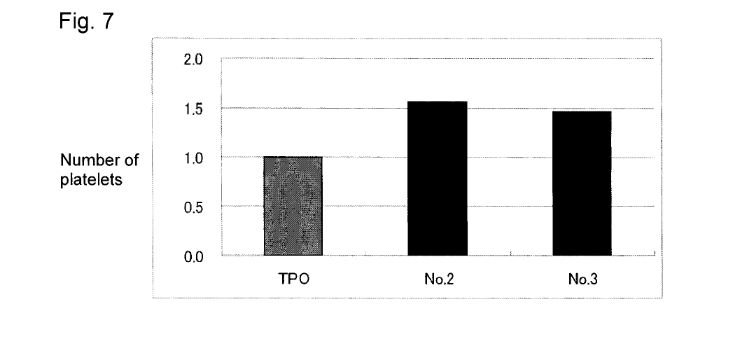

p53 KD-Myc-Bmi-BCLXL cells were seeded on C3H10T1/2 cells inactivated by preliminary treatment with 10 μg/mL mitocycin C (Wako Pure Chemical Industries) for 0.5 to 5 hours (6˜8×10⁵ cells/6-well plate) and cultured in IMDM (Invitrogen/GIBCO) supplemented with 15% FBS (Invitrogen/GIBCO), 2 mM L-glutamine-100 Unit/mL Penicillin-100 μg/mL Streptmysin (Invitrogen/GIBCO), ITS suplement (10 μg/mL insulin, 5.5 mg/mL transferin, 5 ng/mL sodium selenite) (Invitrogen/GIBCO), 50 μg/mL ascorbic acid (Sigma), 0.45 mM monothioglycerol (MTG, Sigma), 10 μg/mL doxycyclin (Clontech), 0.5 mM valproic acid (Sigma), 10 μM Y-27632 (Wako Pure Chemical Industries), 5 μM (S)(−/−)-Blebbistatin (Toronto Research Chemicals), 50 ng/mL SCF (R&D system) and 100 ng/mL human TPO(R&D system) or a specific compound mentioned in Test Example 2 (100 ng/mL No.2 or 50 ng/mL No.3) in 5% CO₂ at 39° C. for 7 days to induce mature megakaryocytes and platelets. The nonadherent cells in the 7-day cultures were characterized by cell surface antigens with a flow cytometer (Becton, Dickinson and Company, BDFACSAria) after addition of 8.5 mM sodium citrate (Sigma), 6.5 mM citric acid (Sigma), 10.4 mM glucose (Sigma), anti-human CD41a antigen (Becton, Dickinson and Company), anti-human CD42b antibody (BioLegend) in terms of final concentration. Platelets were sorted out by size with a flow cytometer and counted by using BD Trucount tubes (Becton, Dickinson and Company). The platelets were positive for the cell surface antigens specific to platelets, human CD41a (integrin αIIb) and human CD42b (GPIbα) (FIG. 7; platelets). The specific compounds of the present invention showed higher thrombopoietic effect than TPO did.

These results demonstrate that megakaryocytes and platelets are induced efficiently from human iPS cells and ES cells in accordance with the method of the present invention by using the specific compounds of the present invention.

### INDUSTRIAL APPLICABILITY

Megakaryocytes and platelets can be expanded from human pluripotent stem cells more efficiently in the presence of a specific compound of the present invention as an active ingredient in culture than in its absence or in the presence of TPO. Platelets produced by using a specific compound are useful for diseases accompanied by a decrease in platelets such as hematopoietic dysfunction and tumors, and hence their application to transfusion therapy is expected. According to the present invention, it is possible to provide platelets which can overcome the problem of HLA compatibility. Therefore, it is possible to supply platelets to patients who require transfusion and solve the problem of platelet destruction by generation of anti-platelet antibodies.

The entire disclosure of Japanese Patent Application No. 2011-219545 filed on Oct. 3, 2011 including specification, claims, drawings and summary is incorporated herein by reference in its entirety.

1. A method for producing a megakaryocyte, a platelet, or both, comprising:
culturing a hematopoietic progenitor cell derived from a pluripotent stem cell ex vivo in the presence of a compound of formula (I), a tautomer, prodrug or pharmaceutically acceptable salt of the compound or a solvate thereof; and
differentiating the hematopoietic progenitor cell into a megakaryocyte, a platelet, or both, 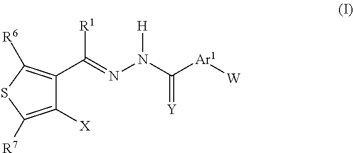
wherein W is a substituent of formula (Ia) or a carboxy group: 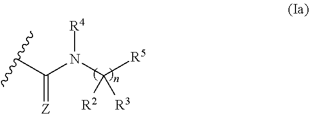
R¹, R², R³ and R⁴ are each independently a C₁₋₁₀ alkyl group which may be substituted with a halogen atom or a hydrogen atom,
n is an integer of 0, 1, 2 or 3,
R⁵ is a C₂₋₁₄ aryl group which may be substituted with a substituent independently represented by V¹, where when n is 2, R⁵ is not an unsubstituted pyridyl group,
R⁶ is a C₁₋₁₀ alkyl group which may be substituted with a halogen atom or a hydrogen atom,
R⁷ is a C₂₋₁₄ aryl group which may be substituted with a substituent independently represented by V²,
Ar¹ is a C₂₋₁₄ arylene group which may be substituted with a substituent independently represented by V³,
X is —OR²⁰,
Y and Z are ach independently an oxygen atom or a sulfur atom,
V¹ is —(CH₂)m₁M¹NR⁸R⁹, —(CH₂)m₆NR¹⁶R¹⁷, -M²NR¹⁸(CH₂)m₇R¹⁹ or —C(═O)-(piperazine-1,4-diyl)-U,
V², V³ and V⁴ are each independently a hydroxy group, a protected hydroxy group, an amino group, a protected amino group, a thiol group, a protected thiol group, a nitro group, a cyano group, a halogen atom, a carboxy group, a carbamoyl group, a sulfamoyl group, a sulfo group, a formyl group, a C₁₋₃ alkoxy group which may be substituted with a halogen atom, a C₁₋₁₀ alkyl group which may be substituted with a halogen atom, a C₂₋₆ alkenyl group, a C₂₋₆ alkynyl group, a C₁₋₁₀ alkylcarbonyloxy group, a C₁₋₁₀ alkoxycarbonyl group, a C₁₋₁₀ alkoxy group, a C₁₋₁₀ alkylcarbonyl group, a C₁₋₁₀ alkylcarbonylamino group, a mono- or di-C₁₋₁₀ alkylamino group, a C₁₋₁₀ alkylsulfonyl group, a C₁₋₁₀ alkylaminosulfonyl group, a C₁₋₁₀ alkylaminocarbonyl group, a C₁₋₁₀ alkylsulfonylamino group or a C₁₋₁₀ thioalkyl group,
M¹ and M² are each independently —(C═O)— or —(SO₂)—,
m₁ is an integer of 0, 1 or 2,
m₂, m₃, m₄, m₅, m₆ and m₇ are each independently an integer of 1 or 2,
R⁸ is a hydrogen atom or a C₁₋₃ alkyl group,
R⁹ and U are each independently —(CH₂)m₂OR¹⁶ or —(CH₂)m₄NR¹¹R¹², provided that when m₁ is 1 or 2, R⁹ may be any of those mentioned above or a hydrogen atom,
R¹⁰ is a hydrogen atom, a C₁₋₃ alkyl group or —(CH₂)m₃T,
R¹¹ and R¹² are each independently a hydrogen atom or —(CH₂)m₅Q, or N, R¹¹ and R¹² mean, as a whole, a substituent of formula (II): 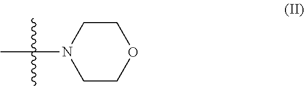 [SMILES: *N1CCOCC1]
or a substituent of formula (III): 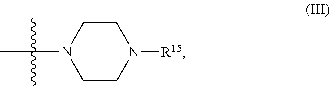
T is a hydroxy group, a C₁₋₆ alkoxy group or a C₁₋₆ alkyl group,
Q is a hydroxy group, a C₁₋₃ alkoxy group or —NR¹³R¹⁴,
R¹³ and R¹⁴ are each independently a hydrogen atom or a C₁₋₃ alkyl group,
R¹⁵ is a hydrogen atom, a C₁₋₃ alkyl group or an amino-protecting group,
R¹⁶ and R¹⁷ are each independently a hydrogen atom, a C₁₋₃ alkylcarbonyl group or a C₁₋₃ alkylsulfonyl group,
R¹⁸ is a hydrogen atom or a C₁₋₃ alkyl group,
R¹⁹ is a C₂₋₉ heterocyclyl group or a C₂₋₁₄ aryl group, and
R²⁰ is a hydrogen atom, a C₁₋₁₀ alkyl group which may be substituted with a substituent independently represented by V⁴ or a C₁₋₁₀ alkylcarbonyl group which may be substituted with a substituent independently represented by V⁴.

2. The method according to claim 1, wherein W is a substituent of formula (Ia):

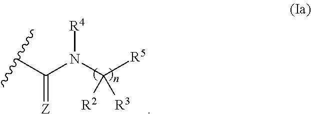

3. The method according to claim 2, wherein R¹ is a hydrogen atom or a C₁₋₆ alkyl group which may be substituted with a halogen atom,
R², R³, R⁴ and R⁶ are each independently a hydrogen atom or a C₁₋₃ alkyl group,
n is an integer of 1 or 2,
Ar¹ is of formula (IV): 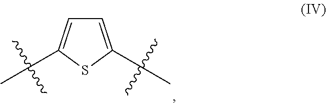 [SMILES: *c1ccc(*)s1]
R⁷ is a phenyl group which may be substituted with at least one substituent selected from the group consisting of C₁₋₁₀ alkyl groups which may be substituted with a halogen atom, C₁₋₁₀ alkoxy groups, C₁₋₃ alkoxy groups substituted with a halogen atom and halogen atoms,
X is —OH, and
Y and Z are oxygen atoms.

4. The method according to claim 3, wherein R², R³, R⁴ and R⁶ are hydrogen atoms.

5. The method according to claim 2, wherein R⁵ is a phenyl group which may be substituted with a substituent independently represented by V¹.

6. The method according to claim 2, wherein R⁵ is a C₂₋₉ heteroaryl group which may be substituted with a substituent independently represented by V¹.

7. The method according to claim 6, wherein the C₂₋₉ heteroaryl group is a C₂₋₉ nitrogen-containing heteroaryl group.

8. The method according to claim 7, wherein the C₂₋₉ nitrogen-comprising heteroaryl group is selected from the group consisting of a 2-pyridyl group, a 3-pyridyl group, a 4-pyridyl group, a 3-pyridazinyl group, a 4-pyridazinyl group, a 2-pyrimidinyl group, a 4-pyrimidinyl group, a 5-pyrimidinyl group and a 2-pyrazinyl group.

9. The method according to claim 7, wherein the C₂₋₉ nitrogen-containing heteroaryl group is a 4-pyridyl group.

10. The method according to claim 2, wherein V¹ is any one of formulae (V) to (XXII):

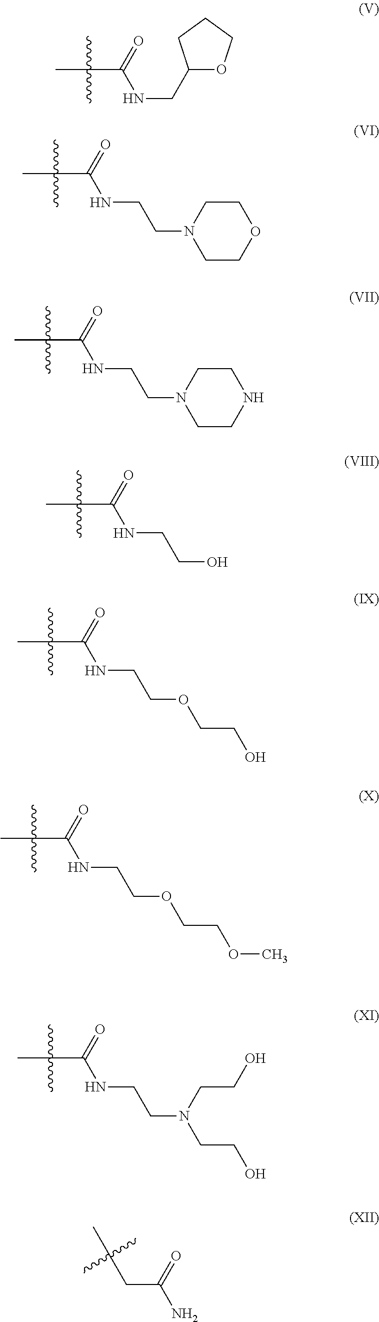

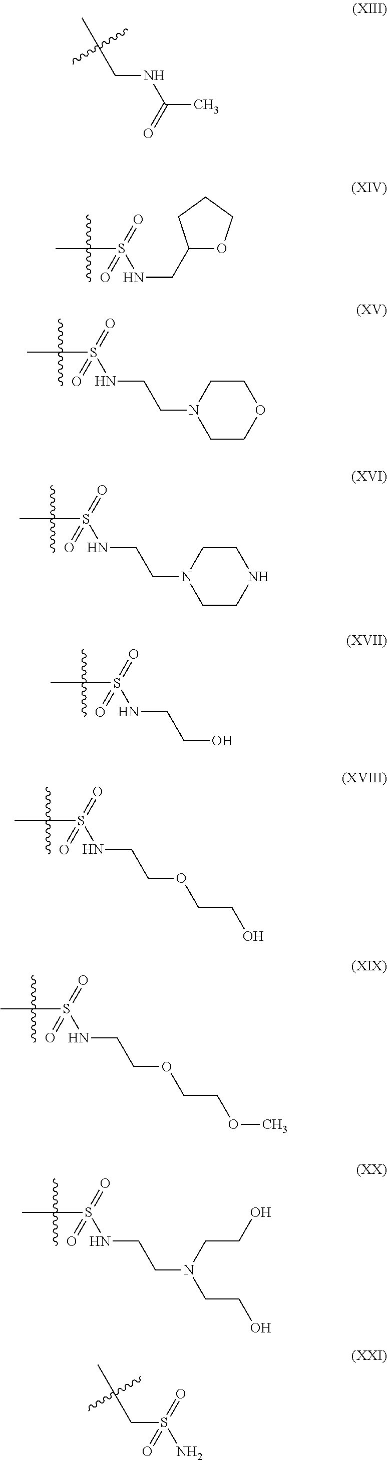

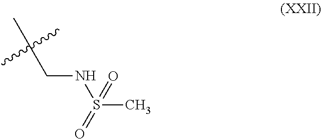 [SMILES: *CNS(C)(=O)=O]

11. The method according to claim 3, wherein R⁵ is a phenyl group substituted with a substituent of formula (VIII):

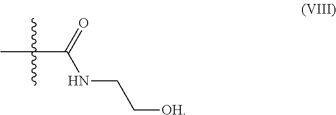 [SMILES: *C(=O)NCCO]

12. The method according to claim 3, wherein R⁵ is a 4-pyridyl group.

13. The method according to claim 2, wherein n is an integer of 1.

14. The method according to claim 2, wherein R⁷ is a phenyl group substituted with at least one substituent selected from the group consisting of methyl groups, t-butyl groups, halogen atoms, methoxy groups, trifluoromethyl groups and trifluoromethoxy groups.

15. The method according to claim 2, wherein R⁷ is a phenyl group which may be substituted with one or two halogen atoms.

16. The method according to claim 2, wherein R¹ is a methyl group.

17. The method according to claim 2, wherein the compound of formula (I) is (E)-5-(2-{1-[5-(3,4-dichlorophenyl)-4-hydroxythiophen-3-yl]ethylidene}hydrazinecarbonyl)-N-[4-(2-hydroxyethylcarbamoyl)benzyl]thiophene-2-carboxamide, (E)-5-(2-{1-[5-(4-bromophenyl)-4-hydroxythiophen-3-yl]ethylidene}hydrazinecarbonyl)-N-[4-(2-hydroxyethylcarbamoyl)benzyl]thiophene-2-carboxamide or (E)-5-(2-{1-[5-(3,4-dichlorophenyl)-4-hydroxylthiophen-3-yl]ethylidene}hydrazinecarbonyl)-N-(pyridin-4-ylmethyl)thiophene-2-carboxamide.

18. The method according to claim 1, wherein W is a carboxy group.

19. The method according to claim 18, wherein R¹ is a hydrogen atom or a C₁₋₆ alkyl group which may be substituted with a halogen atom,
R⁶ is a hydrogen atom or a C₁₋₃ alkyl group which may be substituted with a halogen atom,
R⁷ is a C₂₋₁₄ aryl group
X is —OH,
Y is an oxygen atom or a sulfur atom, and
Ar¹ is of formula (IV):

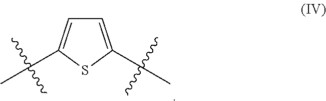 [SMILES: *c1ccc(*)s1]

20. The method according to claim 19, wherein R¹ is a hydrogen atom or a C₁₋₆ alkyl group,
R⁶ is a hydrogen atom,
R⁷ is a substituent of any one of formulae (A01) to (A15): 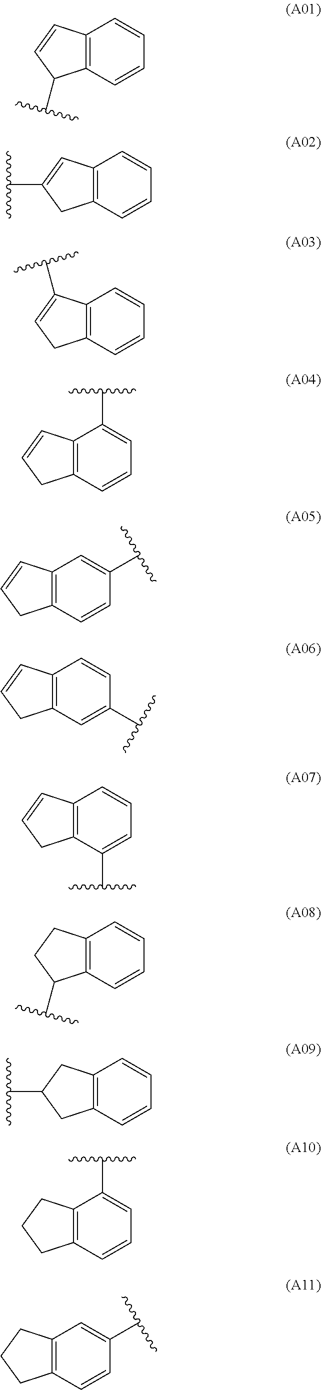 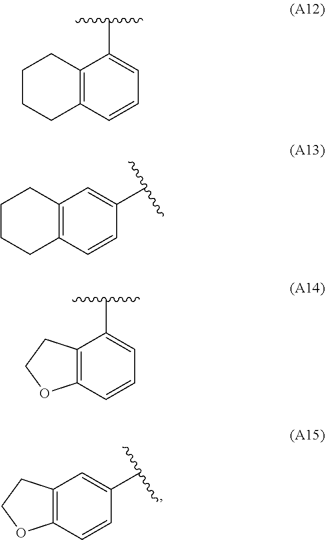 [SMILES: *c1cccc2c1CCCC2 ; *c1ccc2c(c1)CCCC2 ; *c1cccc2c1CCO2 ; *c1ccc2c(c1)CCO2]
and
Y is an oxygen atom.

21. The method according to claim 1, wherein the compound of formula (I) is (E)-5-(2-{1-[5-(2,3-dihydro-1H-indene-5-yl)-4-hydroxythiophen-3-yl]ethylidene}hydrazinecarbonyl)thiophene-2-carboxylic acid.

22. The method according to claim 1, wherein R¹ is a hydrogen atom or a C₁₋₆ alkyl group which may be substituted with a halogen atom,
R², R³, R⁴ and R⁶ are each independently a hydrogen atom or a C₁₋₃ alkyl group,
n is an integer of 1 or 2,
R⁵ is a phenyl group or a C₂₋₉ heteroaryl group which may be substituted with a substituent independently represented by V¹,
R⁷ is a phenyl group which may be substituted with at least one substituent selected from the group consisting of C₁₋₁₀ alkyl groups which may be substituted with a halogen atom, C₁₋₁₀ alkoxy groups, C₁₋₃ alkoxy groups substituted with a halogen atom and halogen atoms or a substituent of any one of formulae (A01) to (A15): 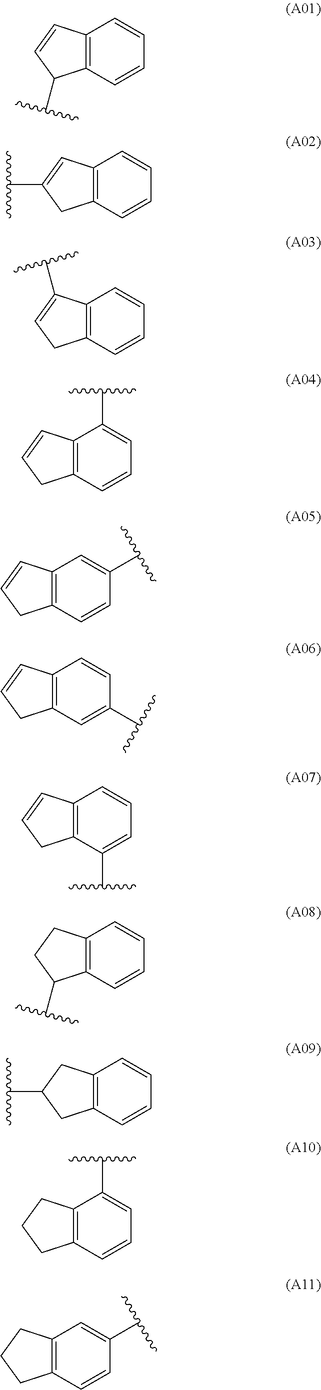 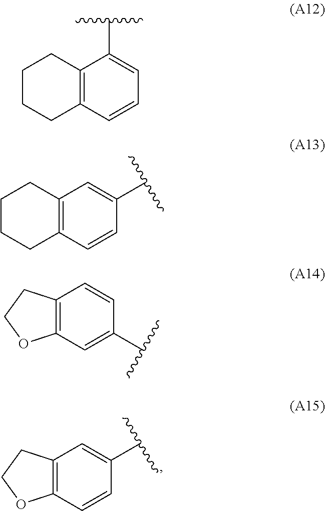
Ar¹ is represented by the formula (IV): 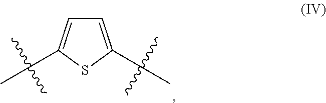 [SMILES: *c1ccc(*)s1]
X is —OH, and
Y and Z are each independently an oxygen atom or a sulfur atom.

23. The method according to claim 22, wherein R¹ is a hydrogen atom or a C₁₋₆ alkyl group,
R², R³, R⁴ and R⁶ are hydrogen atoms,
n is an integer of 1,
R⁵ is a pyridyl group, a pyrazinyl group or a phenyl group substituted with a substituent of formula (VII), (VIII), (XI) or (XII): 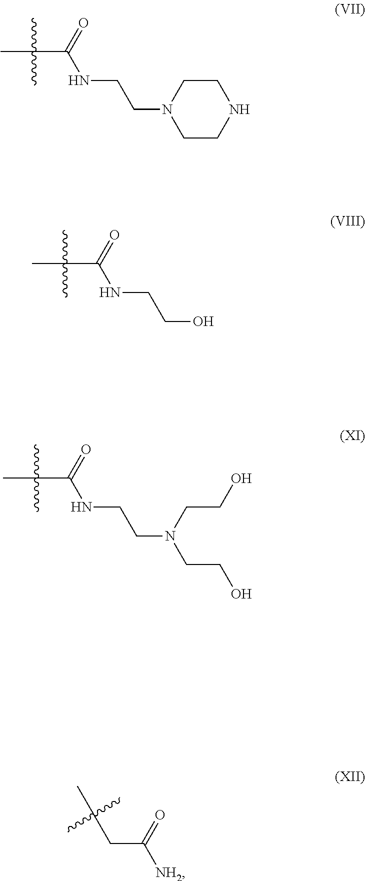 [SMILES: *C(=O)NCCN1CCNCC1 ; *C(=O)NCCO ; *C(=O)NCCN(CCO)CCO ; *CC(N)=O]
R⁷ is a phenyl group which may be substituted with a halogen atom or C₁₋₁₀ alkyl groups or a substituent of formula (A11), (A13) or (A15): 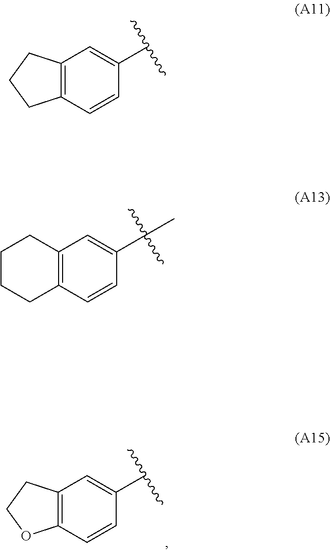 [SMILES: *c1ccc2c(c1)CCC2 ; *c1ccc2c(c1)CCCC2 ; *c1ccc2c(c1)CCO2]
and
Y and Z are oxygen atoms.

24. The method according to claim 1, wherein the compound of formula (I) is (E)-5-(2-{1-[5-(3,4-dichlorophenyl)-4-hydroxythiophen-3-yl]ethylidene}hydrazinecarbonyl)-N-[4-(2-hydroxyethylcarbamoyl)benzyl]thiophene-2-carboxamide, (E)-5-(2-{1-[5-(4-bromophenyl)-4-hydroxythiophen-3-yl]ethylidene}hydrazinecarbonyl)-N-[4-(2-hydroxyethylcarbamoyl)benzyl]thiophene-2-carboxamide, (E)-5-(2-{1-[5-(3,4-dichlorophenyl)-4-hydroxythiophen-3-yl]ethylidene}hydrazinecarbonyl)-N-(pyridin-4-ylmethyl)thiophene-2-carboxamide, (E)-5-(2-{1-[5-(2,3-dihydro-1H-inden-5-yl)-4-hydroxythiophen-3-yl]ethylidene}hydrazinecarbonyl)thiophene-2-carboxylic acid, potassium (E)-2-(3,4-dichlorophenyl)-4-[1-(2-{5-[(pyrazin-2-ylmethyl)carbamoyl]thiophene-2-carbonyl}hydrazono)ethyl]thiophen-3-olate, (E)-5-(2-{1-[5-(4-bromophenyl)-4-hydroxythiophen-3-yl]ethylidene}hydrazinecarbonyl)-N-{4-[2-(piperazin-1-yl)ethylcarbamoyl]benzyl}thiophene-2-carboxamide, (E)-N-[4-(2-amino-2-oxoethyl)benzyl]-5-(2-{1-[5-(3,4-dichlorophenyl)-4-hydroxythiophen-3-yl]ethylidene}hydrazinecarbonyl)thiophene-2-carboxamide or (E)-N-(4-{2-[bis(2-hydroxyethyl)amino]ethylcarbamoyl} benzyl)-5-(2-{1-[5-(4-t-butylphenyl)-4-hydroxythiophen-3-yl]ethylidene}hydrazinecarbonyl)thiophene-2-carboxamide.

25. The method according to claim 1, wherein the pluripotent stem cell is an ES cell or iPS cell.

26. The method according to claim 1, wherein the hematopoietic progenitor cell derived from the pluripotent stem cell is a hematopoietic progenitor cell obtained from a sac-like structure formed by differentiating a pluripotent stem cell into a hematopoietic progenitor cell.

27. The method according to claim 1, wherein the hematopoietic progenitor cell derived from the pluripotent stem cell has at least one introduced gene selected from the group consisting of oncogene, polycomb gene, apoptosis suppressor gene and a gene which suppresses a tumor suppressor gene and has proliferative capability, differentiative capability, or both, enhanced by regulation of expression of the introduced genes.

28. The method according to claim 1, wherein the hematopoietic progenitor cell derived from the pluripotent stem cell is a hematopoietic progenitor cell which has at least one introduced gene selected from the group consisting of MYC family gene, Bmi1 gene, BCL2 family gene and a gene which suppress a p53 gene expression and has proliferative capability, differentiative capability, or both, enhanced by regulation of expression of the introduced genes.

29. A megakaryocyte, platelet, or both, obtained by the method according to claim 1.

30. A blood preparation comprising a platelet obtained by the method according to claim 1, as an active ingredient.

31. A kit suitable for producing a platelet by the method according to claim 1.
